(19) United States
(12) Patent Application Publication
(10) Pub. No.: US 2020/0392497 A1
(43) Pub. Date: Dec. 17, 2020

(54) METHODS AND COMPOSITIONS FOR DETECTING AND PROMOTING CARDIOLIPIN REMODELING AND CARDIOMYOCYTE MATURATION AND RELATED METHODS OF TREATING MITOCHONDRIAL DYSFUNCTION

(71) Applicant: University of Washington, Seattle, WA (US)

(72) Inventors: Jason Wayne Miklas, Seattle, WA (US); Hannele Ruohola-Baker, Seattle, WA (US); Yuliang Wang, Seattle, WA (US)

(73) Assignee: University of Washington, Seattle, WA (US)

(21) Appl. No.: 16/770,767

(22) PCT Filed: Dec. 7, 2018
(86) PCT No.: PCT/US2018/064457

§ 371 Date: Jun. 8, 2020

Related U.S. Application Data
(60) Provisional application No. 62/674,978, filed on May 22, 2018.
(60) Provisional application No. 62/596,438, filed on Dec. 8, 2017.

(51) Int. Cl. C12N 15/113; C12N 5/077; G01N 33/50; C12N 9/22; A61K 35/34

(52) U.S. Cl. CPC C12N 15/113; C12N 5/0657; G01N 33/5091; C12N 2310/20; A61K 35/34; C12N 2800/80; C12N 9/22

(57) ABSTRACT

Embodiments of the disclosure relate to methods and compositions for inducing maturation of cardiomyocytes. In some embodiments, the cardiomyocytes are derived from stem cells, in vitro. In some embodiments, the compositions and methods induce maturation by inducing overexpression of a Let7i microRNA (miRNA), overexpression of miR-452, reduced expression of miR-122, and/or reduced expression of miR-200a in the cardiomyocyte. In other embodiments, the disclosure relates to methods for treating conditions characterized by mitochondrial dysfunction, such as fatty acid oxidations disorders. In other embodiments, the disclosure relates to methods of screening for compounds that affect heart muscle function. In yet other embodiments, the disclosure relates to methods for detecting or monitoring mitochondrial dysfunction in a cell by detecting or monitoring the cardiolipin profile of the cell.

### CROSS-REFERENCES TO RELATED APPLICATIONS

This application claims the benefit of U.S. Provisional Application No. 62/596,438, filed Dec. 8, 2017, and U.S. Provisional Application No. 62/674,978, filed May 22, 2018, both of which is incorporated herein by reference in their entireties.

### STATEMENT OF GOVERNMENT LICENSE RIGHTS

This invention was made with Government support under Grant Nos. P01 GM081619 and R01 GM083867 and R01 GM097372 and R01 HL135143 and U01 HL099993 and U01 HL099997, awarded by the National Institutes of Health. The government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 67910_Sequence_Listing_Final_2018-12-07.txt. The text file is 11 KB; was created on Dec. 7, 2018; and is being submitted via EFS-Web with the filing of the specification.

### BACKGROUND

Mitochondrial trifunctional protein (MTP/TFP) deficiency is thought to be a result of impaired fatty acid oxidation (FAO) due to mutations in hydroxyacyl-CoA dehydrogenase/3-ketoacyl-CoA thiolase/enoyl-CoA hydratase subunit A (HADHA/LCHAD) or subunit B (HADHB). A major phenotype of MTP-deficient newborns is sudden infant death syndrome (SIDS), which manifests after birth once the child begins nursing on lipid-rich breast milk. Defects in FAO have a role in promoting a pro-arrhythmic cardiac environment, however, the exact mechanism of action is not understood, and there are no current therapies.

Pluripotent stem cell derived cardiomyocytes (hPSC-CM) provide a means to study human disease in vitro but are limited due to their immaturity as they are representative of fetal cardiomyocytes (FCM) instead of adult cardiomyocytes (ACM). Due to the lack of knowledge in how committed cardiomyocytes transition from an immature FCM to a mature ACM, many cardiac diseases with postnatal onset have been poorly characterized. During cardiogenesis, FCMs go through developmental states and once past cardiomyocyte commitment exhibit: exit of cell cycle, cessation of spontaneous beating, utilization of lactate, and then at the post-natal stage utilization of fatty acids as the principal energy source and cardiolipin maturation. Since immature hPSC-CMs are unable to utilize fatty acids through FAO as an energy source, they are limited in their use to model FAO disorders.

Current approaches to mature hPSC-CMs toward ACM focus on prolonged culture physically stimulating the cells with either electrical or mechanical stimulation or by 2D surface pattern cues to direct cell orientation.

Notwithstanding the advances in the study of cardiogenesis and mitochondrial trifunctional protein (MTP/TFP), there remains a need to develop adult cardomyocytes to facilitate models for studying cardiac diseases with postnatal onset. The present disclosure addresses this and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method for inducing maturation of cardiomyocyte. The method comprises inducing in an immature cardiomyocyte two or more of the following: overexpression of a Let7i microRNA (miRNA), overexpression of miR-452, reduced expression of miR-122, and reduced expression of miR-200a. In one embodiment, the method comprises inducing in an immature cardiomyocyte overexpression of a Let7i miRNA, overexpression of miR-452, reduced expression of miR-122, and reduced expression of miR-200a.

In another aspect, the disclosure provides the cardiomyocyte produced by any method described herein.

In another aspect the disclosure provides a method of treating a subject with a condition treatable by administration of cardiomyocytes with a mature cardiolipin profile. The method comprising administering to the subject an effective amount of cardiomyocytes as described herein.

In another aspect, the disclosure provides a method of screening a compound for modulation of heart function. The method comprises contacting one or more cardiomyocytes as described herein with a candidate agent; and measuring a cardiac functional parameter in the one or more cardiomyocytes; wherein a change in the cardiac functional parameter indicates the candidate agent modulates heart function.

In another aspect, the disclosure provides a method of treating a mitochondrial fatty acid oxidation (FAO) disorder in a subject. The method comprising administering an effective amount of a composition stabilizing a cardiolipin profile or promoting mature cardiolipin remodeling in mitochondria of the subject.

In another aspect, the disclosure provides a method of detecting the pathological state of a cultured cardiomyocyte. The method comprises determining the cardiolipin profile in the cardiomyocyte, wherein a relative increase of cardiolipins with acyl chains with more than 18 carbons indicates and a relative decrease in cardiolipins with acyl chains with less than 18 carbons indicates a reduced pathological state of the cardiomyocyte.

In yet another aspect, the disclosure provides a composition, or kit of compositions, to induce maturation of a cultured cardiomyocyte. The composition or kit comprise two or more of the following: a nucleic acid construct encoding a Let7i microRNA, a nucleic acid construct encoding miR-452, a nucleic acid construct that is or encodes an oligomer that hybridizes to a portion of a sequence encoding miR-122, and a nucleic acid construct that is or encodes an oligomer that hybridizes to a portion of a sequence encoding miR-200a.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1F illustrate the generation of HADHA Mutant (Mut) and Knockout (KO) stem cell derived cardiomyocytes. FIG. 1A) Schematic of fatty acid beta-oxidation detailing the four enzymatic steps. FIG. 1B) Schematic of HADHA KO DNA and protein sequence from WTC iPSC line showing a 22 bp deletion which resulted in an early stop codon. The illustrated HADHA^{WT} DNA fragment sequence is set forth as SEQ ID NO:1 and the corresponding HADHA^{WT} protein fragment sequence is set forth as SEQ ID NO:2. The illustrated HADHA^{KO} DNA fragment sequence is set forth as SEQ ID NO:3 and the corresponding HADHA^{KO} protein fragment sequence is set forth as SEQ ID NO:4. The Exon, Intron, and In/Del domains are indicated. FIG. 1C) Schematic of HADHA Mut DNA and protein sequence from WTC iPSC line showing a 2 bp deletion and 9 bp insertion on the first allele and a 2 bp deletion on the second allele. The illustrated HADHA^{WT} DNA fragment sequence is set forth as SEQ ID NO:5. The illustrated HADHA^{Mut} DNA fragment sequences are set forth as SEQ ID NO:7 and 9. RNA-Sequencing read counts show that the HADHA Mut expresses exons 4-20 resulting in a truncated protein. FIG. 1D) Western analysis of HADHA expression and housekeeping protein (3-Actin in WTC iPSCs. FIG. 1E) Confocal microscopy of WT, HADHA Mut and HADHA KO hiPSC-CMs for the cardiac marker αActinin (left) and HADHA (right). FIG. 1F) Seahorse analysis trace of fatty acid oxidation capacity of WT, HADHA Mut and HADHA KO hiPSC-CMs.

FIGS. 2A-2F illustrate aspects of the cardiomyocyte maturation microRNA screen. FIG. 2A) Schematic of the workflow performed to determine candidate microRNAs to screen for cardiomyocyte maturation. FIG. 2B) Schematic of the workflow performed to generate microRNA transduced stem cell derived cardiomyocytes. FIG. 2C) Cell area analysis of microRNA treated hiPSC-CMs. MicroRNA-208b OE lead to a significant increase in cell area while miR-205 KO led to a significant decrease. Cells were stained for αActinin, phalloidin, and with DAPI, and imaged with confocal microscopy. FIG. 2D) Micro-electrode array analysis of microRNA treated hiPSC-CMs corrected field potential duration (cFPD). MiR-452 OE led to a longer cFPD. FIG. 2E) Single cell twitch force analysis using a micro-post assay. MiR-200a KO led to a significant increase in twitch force of hiPSC-CMs. FIG. 2F) Seahorse analysis of the maximum change in oxygen consumption rate (OCR) due to FCCP after oligomycin treatment of microRNA treated hiPSC-CMs. MiR-122 KO led to a significant increase in maximum OCR while miR-208b OE, -378e OE and -200a KO led to significant decreases in maximum OCR.

FIGS. 3A-3O illustrate that MiMaC accelerates hiPSC-CM maturation. FIG. 3A) Schematic of the four microRNAs combined to generate MiMaC. FIG. 3B) Single cell force of contraction assay on micro-posts showed that MiMaC treated hiPSC-CMs led to a significant increase in twitch force. FIG. 3C) Representative trace of an EV (control) and a MiMaC treated hiPSC-CM. FIG. 3D) Single cell force of contraction assay on micro-posts showed that MiMaC treated hiPSC-CMs led to a significant increase in power. FIG. 3E) Cell size analysis showed that MiMaC treated hiPSC-CMs led to a significant increase in area. FIG. 3F) Representative confocal microscopy images of EV and MiMaC treated hiPSC-CMs. αActinin (green), phalloidin (red) and DAPI are shown. FIG. 3G) Seahorse analysis of fatty acid oxidation capacity showed that MiMaC treated hiPSC-CMs matured to a point where they could oxidize palmitate for ATP generation while controls cells were not able to utilize palmitate. MiMaC hiPSC-CMs had a significant increase in OCR due to palmitate addition. FIG. 3H) Venn diagram of KO microRNA predicted targets and the identification of HOPX as a common predicted targeted between all KO miRs screened for cardiomyocyte maturation. FIG. 3I) Plot of HOPX expression from RNA-Sequence data during cardiomyocyte maturation. HOPX expression is significantly higher in D30 and 1-year hESC-CMs and 1-year hESC-CMs have statistically significantly higher HOPX as compared to D30 hESC-CMs. * denotes significance vs D20. # denotes significance vs D30. FIG. 3J) HOPX expression in adult human ventricle tissue is significantly higher than fetal human ventricular tissue. Plotted using RNA-sequencing data. FIG. 3K) RT-qPCR of HOPX expression of HOPX showed that MiMaC treated hiPSC-CMs at D30 had a statistically significant higher level of HOPX as compared to EV control D30 hiPSC-CMs. FIG. 3L) Single cell RNA-Seq tSNE plot of unbiased clustering of microRNA treated hPSC-CMs. FIG. 3M) Cluster plot detailing which treatment groups are enriched in each cluster. FIG. 3N) Heatmap of maturation categories based on MiMaC cluster. FIG. 3O) Heatmap of in vivo human maturation markers that are up-regulated with maturation (yellow).

FIGS. 4A-4L illustrate that fatty acid-challenged HADHA Mut CMs displayed elevated cytosolic calcium levels leading to increased beat rate irregularities. FIG. 4A) Seahorse mitostress assay to analyze maximum oxygen consumption rate after oligomycin and FCCP addition. MiMaC treated CMs showed a significant increase in maximum OCR compared to control EV CMs. FIG. 4B) Representative trace of the mitostress assay. FIG. 4C) Seahorse analysis of fatty acid oxidation capacity showed that MiMaC treated hiPSC-CMs matured to a point where they could oxidize palmitate for ATP generation while controls cells were not able to utilize palmitate. MiMaC hiPSC-CMs had a significant increase in OCR due to palmitate addition. Both MiMaC treated Mut and KO hiPSC-CMs were unable to oxidize palmitate. FIG. 4D) Representative trace of the change in fluorescence during calcium transient analysis. FIG. 4E) Quantification of the maximum change in fluorescence during calcium transients. Mut CMs as compared to WT CMs after 12D of Glc+FA media treatment had a statistically significantly lower change in calcium. FIG. 4F) Quantification of the tau-decay constant. Mut CMs as compared to WT CMs after 12D of Glc+FA media treatment had a higher tau-decay constant. FIG. 4G) Representative trace of the change in fluorescence during Fluovolt, action potential, analysis. FIG. 4H) Quantification of the maximum change in fluorescence during action potential. FIG. 4I) Time to wave duration 50% is significantly longer in Mut CMs as compared to WT CMs after 12D of Glc+FA media treatment. FIG. 4J) Representative beat rate trace of Mut CM in Glc or Glc+FA media. FIG. 4K) Quantification of the change in beat interval (ABI). Mut CMs in Glc+FA media as compared to Mut CMs in Glc media had a statistically significant higher ABI. FIG. 4L) Poincaré plot showing ellipses with a 95% confidence interval for each group. The more rounded ellipse of the Mut Glc+FA condition shows that these cells had a greater beat to beat instability as compared to Mut Glc CMs.

FIGS. 5A-5J illustrate that scRNA-Seq revealed multiple disease states of fatty acid challenged HADHA Mut CMs. FIG. 5A) Single cell RNA-sequencing tSNE plot of WT compared to HADHA Mut CMs shows a clear distinction between these two groups. Four conditions of D30 CMs: 6 days of FA treated MiMaC WT CM, 6 days of FA and SS-31 MiMaC WT CMs, 6 days of FA treated MiMaC HADHA Mut CMs and 6 days of FA and SS-31 treated MiMaC HADHA Mut CMs. FIG. 5B) Unbiased clustering revealed 6 unique groups. FIG. 5C) Heatmap detailing the enrichment of conditions in each cluster. FIG. 5D) Heatmap of maturation categories based on MiMaC cluster. FIG. 5E) Heatmap of in vivo mouse maturation markers that are up-regulated with maturation. FIG. 5F) Confocal microscopy showing that HADHA Mut CMs have more nuclei than WT CMs. Blue—DAPI, green—ATP synthase beta subunit and pink—Titin. Inset is of the nuclei shown in grey scale. FIG. 5G) Histogram of the frequency of cells with either 1, 2, 3 or 4 or more nuclei. HADHA mutant CMs have a significant number of cells with 3 or more nuclei. FIG. 5H) Down-regulated metabolic pathways in cluster 0 (non-replicating HADHA CMs) as compared to cluster 3 (WT CMs). FIGURE SI) Down-regulated metabolic pathways in cluster 2 (endoreplicating HADHA CMs) as compared to cluster 3 (WT CMs). FIG. 5J) Up-regulated metabolic pathways in cluster 2 (endoreplicating HADHA CMs) as compared to cluster 3 (WT CMs). Metabolic bubble plot circle size is proportional to the statistical significance. The smaller the p-value, the larger circle. Adjusted p-value 0.01 used as cut-off.

FIGS. 6A-6H illustrate that fatty acid challenged HADHA Mut CMs displayed swollen mitochondria with severe mitochondrial dysfunction. FIG. 6A) Representative confocal images of WT and Mut CMs in 12D of Glc+FA media. FIG. 6B) Quantification of mitotracker and ATP synthase β colocalization and intensity. FIG. 6C) Transmission electron microscopy images of WT and Mut CMs after 12D of Glc+FA media showing sarcomere and mitochondria structure. FIG. 6D) Histogram of mitochondria circularity index for WT and HADHA Mut CMs after 12 days of Glc+FA media showed HADHA Mut CMs mitochondria are rounder. FIG. 6E) Histogram of mitochondria area for WT and HADHA Mut CMs after 12 days of Glc+FA media showed HADHA Mut CMs mitochondria are smaller. FIG. 6F) Quantification of maximum OCR from mitostress assay. Mut and KO CMs as compared to WT CMs after 12D of Glc+FA media had a significantly lower max OCR. FIG. 6G) Quantification of ATP production from mitostress assay, calculated as the difference between baseline OCR and OCR after oligomycin. Mut and KO CMs as compared to WT CMs after 12D of Glc+FA media had significantly lower ATP production. FIG. 6H) Quantification of proton leak from mitostress assay, calculated as the difference between OCR after oligomycin and OCR after antimycin & rotenone. Mut and KO CMs as compared to WT CMs after 12D of Glc+FA media had significantly higher proton leak. SS-31 treated Mut CMs after 12D of Glc+FA had a significantly lower proton leak and non-treated Mut CMs.

FIGS. 7A-7K illustrate that fatty acid challenged HADHA KO and Mut CMs have elevated fatty acids and abnormal cardiolipin profiles. FIG. 7A) Model of long-chain FA intermediate accumulation after the first step of long-chain FAO due to the loss of HADHA. FIG. 7B) The sum of all long-chain acyl-carnitines in WT, Mut and KO FA treated hPSC-CMs. FIG. 7C) Amount of physeteric acid in the free fatty acid state in WT, Mut and KO FA treated hPSC-CMs. FIG. 7D) Amount of palmitoleic acid in the free fatty acid state in WT, Mut and KO FA treated hPSC-CMs. FIG. 7E) Amount of oleic acid in the free fatty acid state in WT, Mut and KO FA treated hPSC-CMs. FIG. 7F) Relative amount of tetra[18:2]-CL in WT and HADHA KO CMs treated with either Glc or Glc+FA. FIG. 7G) Cardiolipin profile generated from targeted lipidomics for WT and HADHA KO CMs treated with either Glc or Glc+FA. FIG. 7H) Cardiolipin profile generated from global lipidomics for WT CMs 12D Glc+FA, HADHA Muts CM 6D and 12D Glc+FA and HADHA KO CMs 12D Glc+FA. FIG. 7I) The sum of all CLs that have myristic acid (14:0) in their side chain in WT, HADHA Mut and HADHA KO CM FA treated hPSC-CMs. FIG. 7J) The sum of all CLs that have palmitic acid (16:0) in their side chain in WT, HADHA Mut and HADHA KO CM FA treated hPSC-CMs. FIG. 7K) Schematic diagram of how HADHA works in series with TAZ to remodel CL.

FIG. 8 graphically illustrates cardiolipin maturation in CM. WT iPSC derived CMs shift their CL profile during maturation by decreasing CLs with [14:0],[14:1][16:1] or [16:0] and increasing CLs with acylchains greater than 18 carbons, including the intermediate [18:1][18:2][18:2][20:2], compared to non-matured iPSC derived CM.

### DETAILED DESCRIPTION

This disclosure is based on the inventors' analysis of mitochondrial tri-functional protein deficiency. As described in more detail below, the inventors addressed a major deficiency in current cell models by generating novel stem cell-derived cardiomyocytes from HADHA-deficient human induced pluripotent stem cells (hiPSCs). The inventors developed methods to accelerate the maturation of the cardiomyocytes using an engineered microRNA maturation cocktail that upregulates the epigenetic regulator, homeobox protein (HOPX). Fatty acid (FA) challenged HADHA mutant cardiomyocytes showed aberrant calcium handling, delayed repolarization and erratic beating suggesting a pro-arrhythmic state. These pathological cardiac manifestations were a result of the underlying mitochondrial pathology, which presented as mitochondrial dysfunction due to proton gradient loss and lack of normal cristae of the mitochondria. The mechanism underlying this pathological mitochondrial state was identified as a dysregulation of cardiolipin homeostasis due to the HADHA knockout and consequent the reduction of tetra[18:2] cardiolipin species. These data revealed the essential dual role of HADHA in fatty acid beta-oxidation and as an acyl-transferase in cardiolipin remodeling for cardiac homeostasis.

These studies provide a novel approach to promoting the maturation of cardiomyocytes for therapeutic, experimental modeling, or drug screening applications. Additionally, the underlying observations of CL modeling provide methods of detecting and monitoring the CL remodeling state to infer health or maturation of the cells.

In accordance with the foregoing, in one aspect the disclosure provides a method for inducing maturation of cardiomyocyte. The method comprises inducing in an immature cardiomyocyte two, three, or all of the following: overexpression of a Let7 microRNA (miRNA), overexpression of miR-208b, overexpression of miR-452, reduced expression of miR-122, and reduced expression of miR-200a. The agents that induce the modulated miRNA expression are together referred to herein as a microRNA maturation cocktail (MiMaC).

In some embodiments, the method comprises inducing in an immature cardiomyocyte at least overexpression of a Let7 miRNA and overexpression of miR-452. In another embodiment, the method comprises inducing in an immature cardiomyocyte at least overexpression of a Let7 miRNA and reduced expression of miR-122. In another embodiment, the method comprises inducing in an immature cardiomyocyte at least overexpression of a Let7 miRNA and reduced expression of miR-200a. In another embodiment, the method comprises inducing in an immature cardiomyocyte at least overexpression of miR-452 and reduced expression of miR-122. In another embodiment, the method comprises inducing in an immature cardiomyocyte at least overexpression of miR-452 and reduced expression of miR-200a. In another embodiment, the method comprises inducing in an immature cardiomyocyte at least reduced expression of miR-122 and reduced expression of miR-200a.

In some embodiments, the method comprises inducing in an immature cardiomyocyte at least overexpression of a Let7 miRNA, overexpression of miR-452, and reduced expression of miR-122. In another embodiment, the method comprises inducing in an immature cardiomyocyte at least overexpression of a Let7 miRNA, overexpression of miR-452, and reduced expression of miR-200a. In another embodiment, the method comprises inducing in an immature cardiomyocyte at least overexpression of a Let7 miRNA, reduced expression of miR-122, and reduced expression of miR-200a. In another embodiment, the method comprises inducing in an immature cardiomyocyte at least overexpression of miR-452, reduced expression of miR-122, and reduced expression of miR-200a.

Let7, miR-452, miR-208b, miR-122, and miR-200a are all microRNA's in cardiomyocytes (CM) that are shown herein to have an influence on aspects of maturation if the SM (see experimental discussion below). As described, the manipulation of these miRNAs is shown to influence signaling pathways that leads to more advanced maturation and cardiolipin remodeling in CM. When implemented in cultured (e.g., stem-cell derived) CM, these miRNA manipulations result in CMs that more closely resemble adult cardiomyocytes (ACM).

Let7 is a family of miRNAs that is described in more detail in Kuppusamy, K. T., et al., Let-7 family of microRNA is required for maturation and adult-like metabolism in stem cell-derived cardiomyocytes. Proc Natl Acad Sci USA, 2015, and U.S. Pat. No. 9,624,471, each of which is incorporated herein by reference in its entirety. The Let-7 miRNA can be selected from Let7a-1, Let7a-2, Let7b, Let7c, Let7e, Let7f-1, Let7f-2, Let7g, and Let7i. In some embodiments, the Let7 miRNA is Let7i. A representative DNA sequence encoding the Let7i miRNA is included within the sequence set forth as SEQ ID NO:11, which is the sequence of an amplicon produced from human genomic template that was inserted into an expression vector to promote expression of the Let7i miRNA. Nucleic acid molecules encoding the indicated Let7 miRNA can be obtained by any conventional approach. In some embodiments, the nucleic acid can be obtained by amplifying the sequence from an encoding genome using specific primers. For example, as described in more detail below, this amplification process was used to amplify and obtain the encoding sequence for Let7i (plus additional sequence up and down stream), such that it could incorporated into an expression vector for overexpression in the cell. Exemplary forward and reverse primers to amplify such a region including Let7i are set forth in SEQ ID NOS:12 and 13, respectively.

A representative sequence encoding miR-452 is included within the sequence set forth as SEQ ID NO:14, which is the sequence of an amplicon produced from human genomic template that was inserted into an expression vector to promote expression of the mi-452 miRNA. Exemplary forward and reverse primers to amplify a region including human miR-452 are set forth in SEQ ID NOS:41 and 42, respectively.

miR-208b is described in, e.g., Callis, T. E., et al., MicroRNA-208a is a regulator of cardiac hypertrophy and conduction in mice. J Clin Invest, 2009. 119(9): p. 2772-86, incorporated herein by reference in its entirety. Exemplary forward and reverse primers to amplify a region including human miR-452 are set forth in SEQ ID NOS:39 and 40, respectively.

A representative sequence encoding miR-122 is included within the sequence set forth as SEQ ID NO:46. A representative sequence encoding miR-200a is included within the sequence set forth as SEQ ID NO:45. Each of these sequences represent amplicons of human genomic sequence that includes the indicated miRNA coding region in addition to additional sequence up and down stream. The sequences of the entire amplicons can be used to transgenically express the entire miRNA. A person of ordinary skill in the could readily use this sequence to generate guide RNAs to hybridize to the encoding sequence, or to generate single stranded nucleic acid fragments that hybridize to a portion of the miRNA, to reduce functional expression of the target miRNA (discussed in more detail below.

With respect to Let7i, miR-452, and miR-208b, the term “induce overexpression” and grammatical variants thereof encompass any additional levels of the miRNA within the cell. In some embodiments, the levels of expression of the target miRNA increase by at least about 1%, 5%, 25%, 50%, 75%, 100%, 125%, 150%, 175%, 200%, 250%, 300%, 350%, 400%, 450%, 500% or more. The overexpression can be induced by enhancing the cell's own endogenous expression activity from its encoding region, or by providing additional exogenous encoding regions to the cell for additional transcription activity. In some embodiments, “induce overexpression” entails simply providing the cell with additional copies of the miRNA itself.

In some embodiments, induction of overexpression of a miRNA (e.g., one or more of Let7i, miR-452, and miR-208b) can comprise a step of contacting the immature CM with a vector comprising a nucleic acid encoding the miRNA to be overexpressed. The vector can be configured to promote either transient or constitutive expression of the miRNA in the cell. In this regard, the nucleic acid is operatively linked to a promoter sequence that can drive the transcription of the miRNA encoding region within the cell. The promoter region can be selected by a person of ordinary skill in the art to accommodate the type of expression desired.

In some embodiments, the vector is configured to promote integration of the nucleic acid encoding the miRNA to be overexpressed (e.g., one or more of Let7i, miR-452, and miR-208b in the same or separate vectors). For example, the vector can be a viral vector comprising the nucleic acid encoding the miRNA to be overexpressed. Any appropriate viral vector for such genome integration of the encoding nucleic acid is contemplated herein. Non-limiting, exemplary viral vectors for this purpose are lentiviral vectors and adeno-associated viral vectors (AAV). Use of a lentiviral embodiment is described in more detail below for illustration.

With respect to miR-122 and miR-200a, the term “reduced expression” encompasses any reduction in the expression levels of functional miRNA within the cell. In some embodiments, reduction in expression levels of a target miRNA encompasses a “sponge” approach wherein a single stranded nucleic acid that hybridizes to at least a portion of the miRNA (i.e., miR-122 or miR-200a) such that it interferes with the capacity of the miRNA to affect transcription of its genomic targets. As indicated above, the sequences encoding the miR-122 and mi-200a are included within the amplicon sequences set forth in SEQ ID NOS:46 and 35, respectively. In this sense, the added nucleic acids “soak” up the target miRNA's from the immature cardiomyocyte and remove them from the milieu of transcription modulators within the cell. In some embodiments, hybridization leads to degradation of the miRNA, such as in RNA interference. The single stranded nucleic acid can be administered directly. In other embodiments, the immature cardiomyocyte cell can be transformed with a sequence encoding the single stranded nucleic acid using a vector configured to promote transient or constitutive expression of the single stranded nucleic acid. Non-limiting examples of vector platforms useful for this purpose include lentiviral and AAV vectors. See the discussion above regarding applicable vectors, which is also applicable in this context.

In other approaches, the miRNA targeted for reduced expression (i.e., miR-122 and/or miR-200a) is targeted for genomic alteration within the immature cardiomyocyte that permanently reduces or knocks out expression of functional target miRNA in the cell. In one embodiment, the immature cardiomyocyte is provided with a guide RNA and a nuclease. The guide RNA has a sequence that allows it to hybridize to a region of the genomic sequence encoding the target miRNA. Upon hybridization, the guide RNA facilitates specific cleavage of genomic region by the nuclease. The immature cardiomyocyte has endogenous DNA repair enzymes that periodically introduce repair mistakes manifesting in a substitution, insertion or deletion within the encoding sequence, resulting in the functional knockout of the miRNA. Even if the repair process is accurate in the initial rounds, eventually, a guide RNA/nuclease/repair combination will result in a misrepair and, thus, functional knockout. Exemplary guide RNAs for miR 122 and miR 200a are discussed below in the Examples and set forth in Table 1 as SEQ ID NOS:17 and 18 (for miR-200a) and SEQ ID NOS:19 and 20 (for miR-122).

In some embodiments, the nuclease has endonuclease activity. Exemplary, non-limiting nucleases include Cas9 and TALENS. Other such nucleases that can specifically edit or cleave DNA based on a guide RNA are known and are encompassed by this disclosure.

The guide RNA can be provided to the immature cardiomyocyte directly or by transgenically expressing the guide RNA in the immature cardiomyocyte. Independent of the guide RNA, the nuclease can be provided to the immature cardiomyocyte directly or by transgenically expressing the nuclease in the immature cardiomyocyte.

In one embodiment, a guide RNA that hybridizes to a region of the genomic sequence encoding the target miRNA is administered directly to the immature cardiomyocyte to facilitate specific cleavage of genomic region by the nuclease. In other embodiments, the guide RNA is transgenically expressed transiently or constitutively in the immature cardiomyocyte by transforming the cell with a nucleic acid construct encoding the guide RNA. An appropriate vector can be selected for the desired expression. For example, non-limiting examples of vector platforms useful to integrate the guide RNA encoding sequence in the immature cardiomyocyte genome include lentiviral and AAV vectors. See the discussion above regarding applicable vectors, which is also applicable in this context.

As indicated above, the nuclease can be provided to the immature cardiomyocyte directly, or can be transgenically expressed transiently or constitutively within the immature cardiomyocyte. To induce expression of the nuclease in the immature cardiomyocyte, the cell can be transformed with a nucleic acid construct encoding the nuclease using a vector configured to promote transient or constitutive expression of the single stranded nucleic acid. For example, non-limiting examples of vector platforms useful to integrate the nuclease encoding sequence in the immature cardiomyocyte genome include lentiviral and AAV vector. See the discussion above regarding applicable vectors, which is also applicable in this context.

In view of the foregoing, illustrative examples of specific embodiments for inducing maturation of cardiomyocyte are described.

In one embodiment, the method comprises inducing in the immature cardiomyocyte reduced expression of one or both of miR-122 and miR-200a. The corresponding guide RNA(s) and nuclease (e.g., Cas9) are transgenically expressed in the immature cardiomyocyte. The DNA encoding the guide RNA (or guide RNAs if both miRNA's are targeted) and the DNA encoding the nuclease can be integrated into the same or separate vectors. Each encoding DNA region is operatively linked to its own promoter sequence configured to drive transcription in the immature cardiomyocyte. The sequence encoding the guide RNA is typically operatively linked to an RNA promoter to ensure that the transcribed guide RNA remains an RNA construct. In some embodiments, DNA encoding the guide RNAs for miR-122 and miR-200a are integrated into the same vector. In other embodiments, the DNA encoding the guide RNAs for miR-122 and miR-200a are integrated into different vectors. In some embodiments, the DNA encoding the nuclease is integrated into the same vector with the DNA encoding the one or both guide RNAs. In other embodiments, the DNA encoding the nuclease is integrated into a different vector with the DNA encoding the one or both guide RNAs.

In some embodiments, cell lines of immature cardiomyocytes are transgenically modified to integrate a gene encoding the nuclease into the cell genome. In this embodiment, the modification can be implemented on the immature cardiomyocyte or a stem cell progenitor thereof. For example, the cell is contacted with a vector (e.g., lentiviral vector) comprising the DNA encoding the nuclease (e.g., Cas9) operatively linked to a promoter, wherein the lentiviral vector permanently integrates the expression cassette with the nuclease gene and promoter into the cell's genome. The promoter can be configured to promote conditional or constitutive expression of the nuclease. With such a genetic background, the immature cardiomyocyte can be contacted with one or more guide RNA's as described above that promote the specific cleavage of the DNA encoding the target miRNA (e.g., miR-122 or miR-200a). The guide RNA's can be produced previously using typical methods (e.g., recombinant expression in bacteria, and the like). This allows for efficient production of a cell culture of cardiomyocytes with knockouts of the desired target miRNAs (e.g., miR-122 and/or miR-200a). Alternatively, the immature cardiomyocyte can be contacted with one or more plasmids or other vectors that incorporate a sequence encoding the guide RNA's as described above that promote the specific cleavage of the DNA encoding the target miRNA (e.g., miR-122 or miR-200a). The DNA encoding the different guide RNAs can be incorporated into the same or different plasmids or other vectors. Integration into the genome is not necessary and transient expression of the guide RNA's can suffice to cause knockout in the cell.

In yet another embodiment, the nuclease (e.g., Cas9) and the guide RNA (e.g., directed to the DNA encoding miR-122 or miR-200a) can be produced exogenously and administered directly to the immature cardiomyocyte without reliance on transgenic expression in the immature cardiomyocyte itself.

In other embodiments where both overexpression of target miRNAs and reduced expression of other target miRNAs are sought, the nucleic acid constructs driving overexpression or reduced expression for each of the respective target miRNA can be integrated into the same vector construct. In exemplary embodiment, the cell is contacted with a vector that comprises an expression cassette with multiple nucleic acid expression constructs. In this embodiment, the cell can be the immature cardiomyocyte or a stem cell progenitor thereof. The expression cassette can promote inducible expression of the transcripts encoded therein, using an inducer specific to the vector. The expression cassette can include any combination of the encoding constructs indicated above. To illustrate, in an embodiment, the expression cassette comprises DNA sequence(s) encoding each of one or more miRNA targeted for overexpression (e.g., one or more of Let7, miR-452, and miR-208b) as well as DNA sequence(s) encoding the single stranded nucleic acid(s) that hybridize with the one or more miRNAs targeted for reduced expression (e.g., one or both of miR-122 and miR-200a). An exemplary vector applicable to this embodiment is pAC150-PBLHL-4×HS-EF1a-DEST (Addgene, #48234), which has insulator sequence flanking the expression cassette to ensure the expression constructs in the cassette are not silenced. Such an inducible vector can be induced by, e.g., doxycycline, to promote the expression of the members of the MiMaC contained therein.

As indicated above, the immature cardiomyocyte can be derived from a stem cell. In some embodiments, the cell is derived from stem cells in vitro by promoting differentiation of the stem cell into an immature stem cell, as described in more detail in the Examples. This process is also described in more detail in, e.g., Palpant, N.J., et al., Generating high- purity cardiac and endothelial derivatives from patterned mesoderm using human pluripotent stem cells. Nat Protoc, 2017. 12(1): p. 15-31; Burridge, P. W., et al., Chemically defined generation of human cardiomyocytes. Nat Methods, 2014. 11(8): p. 855-60; and Tohyama, S., et al., Distinct metabolic flow enables large-scale purification of mouse and human pluripotent stem cell-derived cardiomyocytes. Cell Stem Cell, 2013. 12(1): p. 127-37 each of which is incorporated herein by reference in its entirety. The stem cell can be an embryonic stem cell, a pluripotent stem cell, or an induced pluripotent stem cell.

In some embodiments, the method further comprises contacting the immature cardiomyocyte with an effective amount of a long chain fatty acid. As used herein, the term “effective amount” refers to an amount sufficient to promote maturation of the cardiomyocyte and/or cardiolipin remodeling in the cell into a more mature state. In some embodiments, the method further comprises contacting the immature cardiomyocyte with at least two long chain fatty acid species. In some embodiments, the method further comprises contacting the immature cardiomyocyte with at least three long chain fatty acid species. The long chain fatty acid species can be selected from palmitic acid, oleic acid, and linoleic acid. Typically, palmitic acid is used with either oleic acid or linoleic acid because on its own it can be cytotoxic to the cells.

The long chain fatty acids can be contacted to the immature cardiomyocyte in a form wherein it is conjugated to a carrier, such as BSA, that can assist its uptake and stability. Exemplary oleic acid/BSA conjugate concentrations or ranges in the cell culture media include: about 10-14 μg/mL, about 11-13 μg/mL, about 12-13 μg/mL, such as about 11 μg/mL, about 11.5 μg/mL, about 12 μg/mL, about 12.5, μg/mL, about 12.7 m/mL, about 13 μg/mL, and about 13.25 μg/mL. Exemplary linoleic acid/BSA conjugate concentrations or ranges in the cell culture media include: about 5.5-8.5 m/mL, about 6.5-8 m/mL, about 6.75-8.0 μg/mL, such as about 6 μg/mL, about 6.5 μg/mL, about 7 μg/mL, about 7.05, μg/mL, about 7.5 μg/mL, about 8 μg/mL, and about 8.25 μg/mL. Exemplary plamitic acid (in the form of sodium palmitate)/BSA conjugate concentrations or ranges in the cell culture media include: about 40-60 μM, about 45-55 μM, about 50-55 μM, such as about 45 μg/mL, about 48 μM, about 50 μM, about 52.5 μM, about 55 μM, about 58 μM, and about 60 μM. In one exemplary embodiment, as described in the Examples, the fatty acid media utilized concentrations of with oleic acid conjugated to BSA (Sigma 03008): 12.7 μg/mL, linoleic acid conjugated to BSA (Sigma L9530): 7.05 μg/mL, sodium palmitate (Sigma P9767) conjugated to BSA (Sigma A8806): 52.5 μM.

In further embodiments, the method further comprises contacting the immature cardiomyocyte with carnitine at concentrations of about 100-150 μM, such as about 110-140 μM, about 115-135 μM, and about 120-130 μM. Exemplary concentrations include about 100 m/mL, 110 m/mL, about 120 μM, about 125 μM, about 130 μM, about 135 μM, about 140 μM, and about 150 μM. The carnitine assists the transportation of the administered long chain fatty acids into the mitochondria.

In some embodiments, the immature cardiomyocyte comprises a genetic aberration. The genetic aberration can be associated with a metabolic or pathological disease state in the heart. For example, the genetic aberration is associated with a fatty acid oxidation (FAO) disorder. In some embodiments, the cardiomyocyte comprises a mutation in a gene encoding one of the following: HADHA, FATP1, FACS1, OCTN2, L-CPTI, M-CPT I, CAT, CPT II, VLCAD, LCAD, MCAD, SCAD, LCHAD, SHYD, M/SCHAD, SKAT, MKAT, HS, HL, ETF, and ETF QO, which result in a fatty acid disorder. By implementing the genetic aberration in a cardiomyocyte that with induced maturation, the resulting cardiomyocyte provides a disease model for an ACM with the indicated aberration.

In another aspect, the disclosure provides the cardiomyocyte produced by the above methods. As indicated, the cardiomyocyte can be derived from a stem cell, such as an embryonic stem cell, a pluripotent stem cell, or an induced pluripotent stem cell. In some embodiments, the stem cell is from a human.

Furthermore, as described in more detail, the cell can comprise a genetic aberration, such as an aberration associated with a fatty acid oxidation (FAO) disorder. Target genes containing exemplary genetic aberrations are listed above. In one embodiment, the genetic aberration is a mutation in the gene encoding HADHA.

Considering the methods of applying the MiMaC to induce maturation in the cardiomyocyte, the cell can comprise exogenous nucleic acids that are or encode miRNAs to be overexpressed (i.e., Let7, miR-452, and/or miR-208b). Alternatively or additionally, the cell can comprise exogenous nucleic acids that are or encode single stranded nucleic acids that can hybridize to a target miRNA targeted for reduced expression (i.e., miR-122 and/or miR-200a). Alternatively or additionally, the cell can comprise exogenous nucleic acids that are or encode guide RNAs that can hybridize to the genomic sequence encoding the miRNA targeted for reduced expression (i.e., miR-122 and/or miR-200a). In some embodiments that involve guide RNAs for the reduced expression of the target miRNA, the cell also comprises a nuclease (e.g., Cas9 or TALENS) or a nucleic acid construct encoding the nuclease. In one embodiment, the cell comprises an expression cassette with a first nucleic acid encoding a Let7 miRNA, a second nucleic acid encoding miR-452, a third nucleic acid encoding a single stranded nucleic acid that hybridizes to at least a portion of miR-122, and a fourth nucleic acid that encodes a single stranded nucleic acid that hybridizes to at least a portion of miR-200a. The nucleic acid sequences are operably linked to one or more promoters. In a further embodiment, expression of the nucleic acid sequences can be induced from the application of doxycylin.

In another aspect, the disclosure provides a method of treating a subject with a condition treatable by administration of cardiomyocytes with a mature cardiolipin profile. The method comprising administering to the subject an effective amount of cardiomyocytes produced by the method described herein to promote maturation in culture. For example, the method can comprise culturing inducing stem cells obtained from the subject to differentiate into immature cardiomyocytes, administer the MiMaC to the cells, in any format described herein, and permitting the cells to progress in their maturation towards adult cardiomyocytes. The cells can then be administered to the subject in need. In other embodiments, the stem cells can be from a different subject of the same species. As indicated above, the stem cells can be embryonic, pluripotent, or induced pluripontent stem cells.

The subject can be any mammal. In some embodiments, the subject is a rodent or primate. In some embodiments, the subject is human, dog, cat, mouse, rat, rabbit, and the like.

In some embodiments, the subject has compromised cardiac cells in heart tissue. This can include scenarios where the subject has diabetes, congenital heart disease, ischemia, myopathy, mitochondrial disease, and/or has suffered from infarction events. In some embodiments, the mitochondrial disease is a fatty acid oxidation (FAO) disorder. In some embodiments, the subject has mitochondrial trifunctional protein (MTP/TFP) deficiency. In some embodiments the subject has a mutation in the gene encoding HADHA. In other embodiments, the subject has a mutation in a gene encoding at least one of FATP1, FACS1, OCTN2, L-CPTI, M-CPT I, CAT, CPT II, VLCAD, LCAD, MCAD, SCAD, LCHAD, SHYD, M/SCHAD, SKAT, MKAT, HS, HL, ETF, and ETF QO.

In some embodiments, the condition or dysfunction can manifest in experiencing arrhythmia. The subject, can be a newborn or infant with high risk of sudden infant death syndrome (SIDS), such as in the case of, e.g., having mitochondrial trifunctional protein (MTP/TFP) deficiency.

The cells can be readily formulated for administration to damaged heart tissue according to techniques understood in the art.

In another aspect, the disclosure provides a method of treating a mitochondrial fatty acid oxidation (FAO) disorder in a subject. The method comprises administering an effective amount of a composition stabilizing a cardiolipin profile or promoting mature cardiolipin remodeling in mitochondria of the subject.

The subject can be any mammal, such as a human.

The mitochondrial dysfunction can be associated with diabetes, heart failure, neurodegeneration, advanced age, congenital heart disease, ischemia, myopathy, and/or instance of infarction. In some embodiments, the FAO disorder is a fatty acid β-oxidation disorder. The FAO disorder can be associated with mutations in any of the genes indicated above. In some embodiments, the mitochondrial dysfunction is associated with arrhythmia and/or increased risk of sudden infant death syndrome.

In some embodiments, stabilizing a cardiolipin profile comprises prevention of oxidation of cardiolipin. In some embodiments, the composition is or comprises elamipretide (also referred to as SS-31) (Stealth BioTherapeutics Inc, Newton, Mass.), which is a small mitochondrial-targeted tetrapeptide that is known to reduce the production of toxic reactive oxygen species and stabilize cardiolipin. In one embodiment, an effective amount of elamipretide is administered to a subject with mitochondrial trifunctional protein deficiency.

In another aspect, the disclosure provides a method of screening a candidate compound for potential modulation of heart function. In this regard, the methods and compositions described herein have enabled the production of cultured cardiomyocytes to progress in their maturation to more accurately reflect adult cardiomyocytes. Therefore, such cells can be readily produced in vitro to provide for a screening process of candidate agents/compounds.

The method comprises contacting one or more cardiomyocytes produced by the methods described herein with a candidate agent; and measuring a cardiac functional parameter in the one or more cardiomyocytes. A change in the cardiac functional parameter indicates the candidate agent modulates heart function. A candidate that promotes favorable functional parameters, and/or reduces negative functional parameters can be selected as a strong candidate agent or compound for treatment or continued study.

Cardiac functional parameters can include any relevant, measurable parameter with implications on heart tissue function. Non-limiting, exemplary cardiac functional parameters include the lipid profile, the cardiolipin profile, metabolic profile, oxygen consumption rate, mitochondrial proton gradient, force of contraction, calcium transport, conduction velocity, glucose stress, and cell death in defined circumstances. Furthermore, potential toxicity and dosing concentrations can be tested in the disclosed cells.

In addition to screening a candidate agent for an effect on a model of healthy adult cardiomyocytes, the method also comprises embodiments of screening candidate compounds for the effects of disease models that have a more mature cardiomyocyte status. Thus, in some embodiments, the matured cardiomyocyte used in the screen can comprises a genetic aberration, such as described elsewhere herein. The aberration can be associated, for example, with a fatty acid oxidation (FAO) disorder. To illustrate, the experimental description below addresses cells with a genetic mutation in the HADHA protein. The cells were induced to progress to a more mature state to provide a model of an adult cardiomyocyte with mitochondrial trifunctional protein (MTP/TFP) deficiency. This allowed testing of compounds to counter the dysfunction.

In another aspect, the disclosure provides a method of detecting a pathological state of a cultured cardiomyocyte. The method comprises determining the cardiolipin profile in the cardiomyocyte. A relative increase of cardiolipins with acyl chains with more than 18 carbons indicates and/or a relative decrease in cardiolipins with acyl chains with less than 18 carbons indicates a reduced pathological state of the cardiomyocyte.

An exemplary lipidomics methodology for determining the cardiolipin profile is described in more detail below in the Examples.

The relative increase or decrease of cardiolipins can be in comparison to a reference standard for a cardiomyocyte, such as derived from a wild-type adult cardiomyocyte or a cultured cardiomyocyte established as exhibiting normal or acceptable mitochondrial function, or having an established normal or mature cardiolipin profile. In other embodiments, the relative increase or decrease of cardiolipins can be in comparison to a wild-type immature cardiomyocyte or a cultured cardiomyocyte established as exhibiting normal or acceptable mitochondrial function, or having an established normal or mature cardiolipin profile. The experimental disclosure below describes the profiling of cardiolipins in cultured human cardiomyocytes during the maturation process.

The cultured cardiomyocyte can be derived from a stem cell in vitro, such as an embryonic stem cell, pluripotent stem cell, or induced pluripotent stem cell, as described above.

The pathological state can be a state associated with a mitochondrial dysfunction, as described in more detail above. In some embodiments, the mitochondrial dysfunction is mitochondrial tri-functional protein deficiency.

The method can be performed to ascertain whether cultured cells are sufficiently mature, i.e., have sufficient cardiolipin remodeling, to serve their intended purpose. Additionally, the method can be performed at one or more times during an in vitro screen of a candidate agent compound to ascertain its impact on cardiac homeostasis or other mitochondrial function. Thus, the method can comprise further contacting the cultured cardiomyocyte with a candidate agent for reducing the pathological state of the cultured cardiomyocyte. The timing of the detection steps can be designed appropriately for the particular screen or treatment. The determining step can be performed, for example, a plurality of times before, during, and/or after the step of contacting the cultured cardiomyocyte with a candidate agent to ascertain the effect of the candidate agent on the pathological state of the cultured cardiomyocyte.

It will be appreciated that the determining methodology can also be extended to be performed on cells obtained from a subject to diagnose a pathological state of a cardiomyocyte.

In this regard, mitochondrial trifunctional protein deficiency often manifests coordinately in cells from the heart (cardiomyocytes), liver (hepatocytes), and retina. Thus one or more cells from any of these tissues (typically liver) can be obtained and the cardiolipin profile can be ascertained. Lower relative levels of cardiolipin with 18 (or higher) carbon chains, as described herein, indicate a failure of the cells to fully remodel cardiolipin from an immature to a mature state. Failure of the cardiolipin remodeling indicates an inability for the cell to efficiently utilize fatty acids as the primary energy source. This failure is likely to be experienced in parallel with cardiomyocytes in the subject, leading to an increased risk of pathologies, such as, e.g., arrhythmia and SIDS.

In another aspect, the disclosure provides a composition, or kit of compositions, to induce maturation of a cultured cardiomyocyte. The composition can be useful in the methods, described above, to promote the maturation of an immature cardiomyocyte. The composition addresses the MiMaC formulation and comprises two or more of the following: a nucleic acid construct encoding a Let7i microRNA, a nucleic acid construct encoding miR-452, a nucleic acid construct that is or encodes a nucleic acid fragment that hybridizes to a portion of a sequence encoding miR-122, and a nucleic acid construct that is or encodes a nucleic acid fragment that hybridizes to a portion of a sequence encoding miR-200a.

In one embodiment, the composition comprises three or more of the following: a nucleic acid construct encoding a Let7i microRNA, a nucleic acid construct encoding miR-452, a nucleic acid construct that is or encodes a nucleic acid fragment that hybridizes to a portion of a sequence encoding miR-122, and a nucleic acid construct that is or encodes a nucleic acid fragment that hybridizes to a portion of a sequence encoding miR-200a. In a further embodiment, the composition comprises a nucleic acid construct encoding a Let7i microRNA, a nucleic acid construct encoding miR-452, a nucleic acid construct that is or encodes a nucleic acid fragment that hybridizes to a portion of a sequence encoding miR-122, and a nucleic acid construct that is or encodes a nucleic acid fragment that hybridizes to a portion of a sequence encoding miR-200a.

The target miRNAs are described in more detail above. Also described are the nucleic acid fragments that hybridize to a portion of a sequence encoding a target miRNA so as to prevent functionality and result in a functional reduction of expression.

The nucleic acid constructs that encode a microRNA and/or encode a nucleic acid fragment are each operatively linked to one or more promoter sequences.

One or more of constructs can be incorporated into one or more vectors configured for delivery to a cell. The one or more vectors can be viral vectors, such as lentiviral or AAV vectors.

In some embodiments, each nucleic acid construct is incorporated into an individual vector and, thus, the composition comprises and admixture of multiple vectors (with different incorporated expression constructs). In another embodiment, each of the nucleic acid constructs is incorporated into the same vector. For example, the multiple nucleic acid constructs can be incorporated into the same expression cassette that is incorporated into a single vector. The vector can be configured to promote expression of all the constructs in the cassette, either constitutively or transiently (e.g., by induction). The vector can provide insulator sequences to prevent inactivation after delivery to the cell. An exemplary vector applicable to this embodiment is pAC150-PBLHL-4×HS-EFla-DEST (Addgene, #48234).

The nucleic acid fragment that hybridizes to a portion of a sequence encoding miR-122 and the nucleic acid fragment that hybridizes to a portion of a sequence encoding miR-200a are guide RNA molecules that are configured to induce a gene editing enzyme to cleave miR-122 and miR-200a, respectively. The gene editing enzyme can be a nuclease and/or have endonuclease function, as described above. Examples are Cas9 and TALENS, although others are known and encompassed by this disclosure.

In some embodiments, the kit or composition disclosed herein further comprises the nuclease. In other embodiments, the kit or composition further comprises a nucleic acid construct that encodes the nuclease. The nuclease-encoding nucleic acid construct is operatively linked to a promoter sequence that facilitates the expression of the nuclease in the target cell.

In some embodiments, the kit or composition further comprises one or more long-chain fatty acids, which are described in more detail above. The one or more long-chain fatty acids comprise palmitic acid, oleic acid, and/or linoleic acid. In some embodiments, the kit or composition comprises a combination of palmitic acid, oleic acid, and linoleic acid.

In some embodiments, kits disclosed herein can further comprise cell culture medium and instruction to facilitate preparation of mature cultured cardiomyocytes from the stem-cell derived cardiomyocytes.

In another embodiment, kits disclosed herein can further comprise stem cell-derived cardiomyocytes, which can be metabolically active or frozen. In another embodiment, the kit and/or any of its constituents can be shipped and/or stored at ambient or room temperature, or at, e.g., 4° C. The stem cell-derived cardiomyocytes can be ultimately derived from a subject with a disease or disorder (e.g., mitochondrial dysfunction, as described herein) or are genetically modified to mimic a disease or disorder, including, for example, a cardiac disease or disorder.

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, N.Y. (2001); Ausubel, F. M., et al. (eds.), Current Protocols in Molecular Biology, John Wiley & Sons, New York (2010); Coligan, J. E., et al. (eds.), Current Protocols in Immunology, John Wiley & Sons, New York (2010); Mirzaei, H. and Carrasco, M. (eds.), Modern Proteomics—Sample Preparation, Analysis and Practical Applications in Advances in Experimental Medicine and Biology, Springer International Publishing, 2016; and Comai, L, et al., (eds.), Proteomic: Methods and Protocols in Methods in Molecular Biology, Springer International Publishing, 2017, for definitions and terms of art.

For convenience, certain terms employed herein, in the specification, examples and appended claims are provided here. The definitions are provided to aid in describing particular embodiments and are not intended to limit the claimed invention, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

The following describes a study addressing disease etiology of mitochondrial trifunctional protein (MTP) deficiency, which can lead to sudden infant death syndrome (SIDS). The study involved development of a novel approach to promote maturation of cardiomyocytes (CMs) from induced pluripotent stem cells was developed to generate a model of CMs reflective of relevant disease states.

Abstract

Mitochondrial trifunctional protein deficiency results from mutations in hydratase subunit A (HADHA). To reveal the disease etiology, stem cell-derived cardiomyocytes were generated from HADHA-deficient hiPSCs and accelerated their maturation via a novel, engineered MicroRNA Maturation Cocktail (“MiMaC”) that upregulated the epigenetic regulator, HOPX. Fatty acid challenged MiMaC treated HADHA mutant cardiomyocytes manifested the disease phenotype: defective calcium dynamics and repolarization kinetics which resulted in a pro-arrhythmic state. Single cell RNA-seq revealed a novel cardiomyocyte developmental intermediate, based on metabolic gene expression. This intermediate gave rise to mature-like cardiomyocytes in control cells but, mutant cells transitioned to a pathological state with reduced fatty acid beta-oxidation (FAO), reduced mitochondrial proton gradient, disrupted cristae structure and defective cardiolipin remodeling. This study reveals that TFPa/HADHA, a MLCL-AT-like enzyme, is required for FAO and cardiolipin remodeling, essential for functional mitochondria in human cardiomyocytes.

Results

Generation of Mitochondrial Trifunctional Protein Deficient Cardiomyocytes

To recapitulate the cardiac pathology of mitochondrial trifunctional protein deficiency on the cellular level in vitro, the CRISPR/Cas9 system was used to generate mutations in the gene HADHA of human iPSCs. From the wild type (WT) hiPSC line, which serves as our isogenic control, multiple HADHA mutant hiPSC lines were generated using two different guides targeting exon 1 of HADHA mutations were confirmed for clones by Western blot (not shown). A knockout (KO) HADHA (HADHA^{KO}) and compound heterozygote (HADHA^{Mut}) hiPSC lines that were generated using gRNA1 were used for further study.

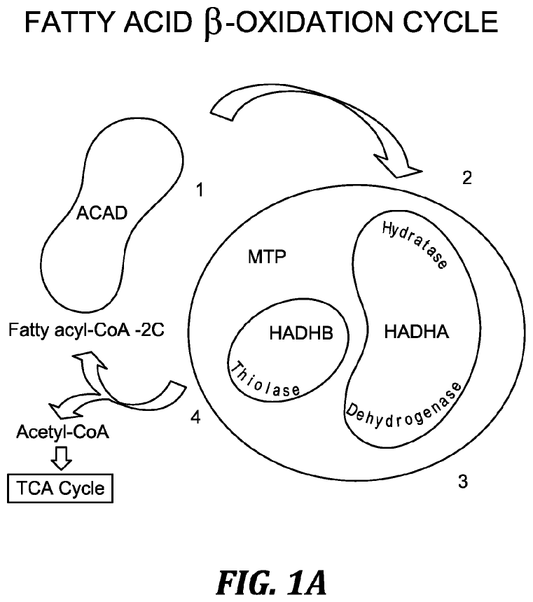

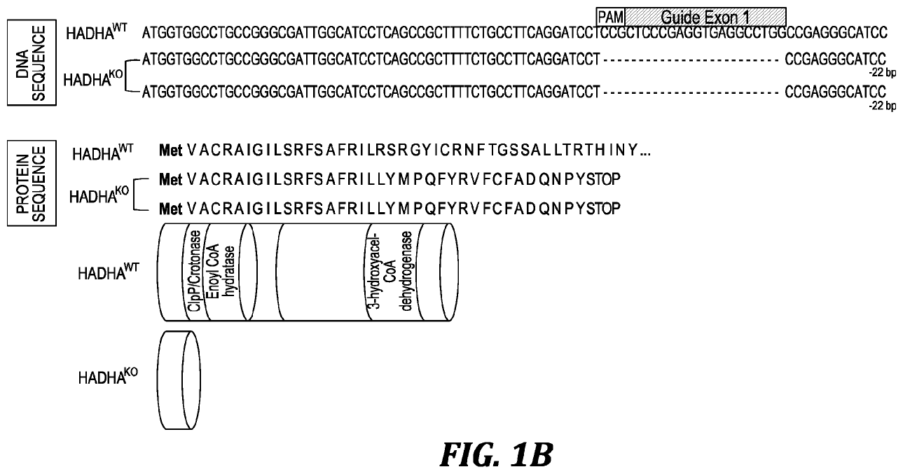

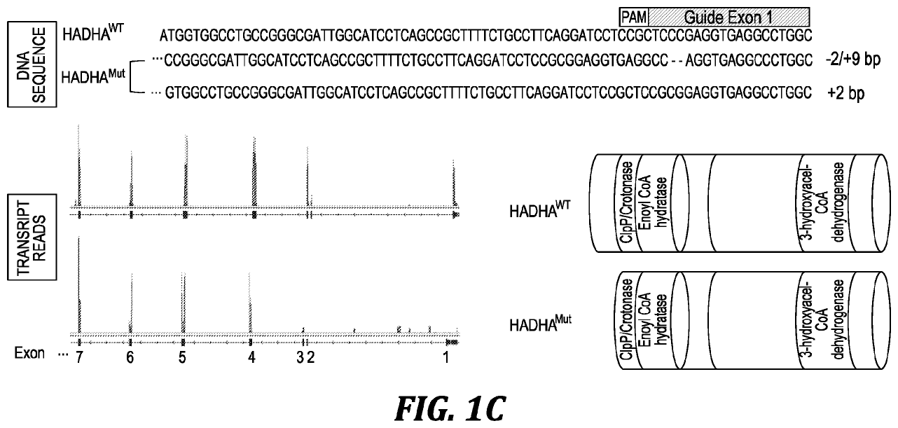

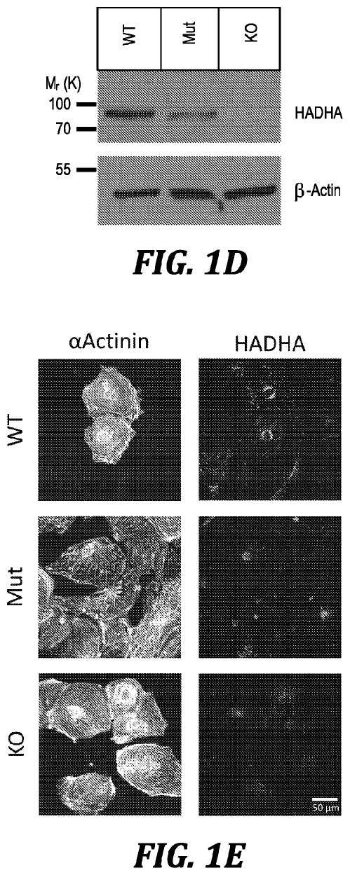

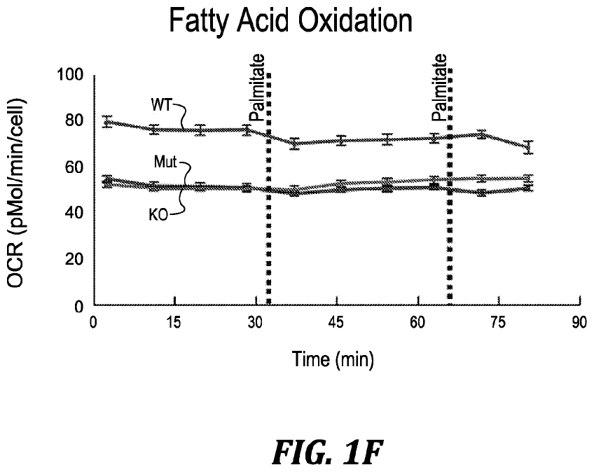

Examining the DNA sequence of the HADHA^{KO} line showed a homozygous 22 bp deletion, which resulted in an early stop codon in exon 1 (FIG. 1B). The HADHA^{Mut} line had a 2 bp deletion and 9 bp insertion on the first allele and a 2 bp insertion on the second allele (FIG. 1C). Both lines showed no off-target mutations on the top three predicted sites (not shown). The mutations found in the HADHA^{Mut} line resulted in a predicted early stop codon on both alleles. (A representative HADHA^{WT} protein fragment sequence corresponding to the wild-type sequence illustrated in FIG. 1C is set forth in SEQ ID NO:6. HADHA^{Mut} protein fragment sequence corresponding the two HADHA^{Mut} mutant alleles illustrated in FIG. 1C are set forth as SEQ ID NOS:8 and 10, respectively). However, when the protein in each line was examined HADHA was observed to be expressed in the WT hiPSC line, not expressed in the HADHA^{KO} line, and was still expressed, to a lower degree, in the HADHA^{Mut} line (FIG. 1D). The transcript of HADHA expressed in WT and HADHA^{Mut} lines was then examined. The WT line was found to express the full length HADHA transcript from exon 1-20 while the HADHA^{Mut} line skipped exons 1-3 and expressed HADHA exons 4-20 (FIG. 1C). It is possible that the mutations generated at the intron-exon junction induced an alternative splicing event and a new transcript as there is no known transcript of HADHA from exon 4-20. The observed reduction in the HADHA mutant molecular weight (FIG. 1D) supports this hypothesis. The expressed HADHA^{Mut} protein skips the expression of exons 1-3, 60 amino acids, generating a truncated ClpP/crotonase domain, which likely compromises the mitochondrial localization and protein folding of the enzyme pocket resulting in the inability to stabilize enolate anion intermediates during FAO.

Using a monolayer directed differentiation protocol [Palpant, N.J., et al., Generating high-purity cardiac and endothelial derivatives from patterned mesoderm using human pluripotent stem cells. Nat Protoc, 2017. 12(1): p. 15-31], a human induced pluripotent stem cell derived cardiomyocytes (hiPSC-CMs) was generated from the three lines. The reduction or loss of HADHA was found to not hinder the ability to generate cardiomyocytes (see images in FIG. 1E). To assess the functional phenotype of the MTP deficient cardiomyocytes, a Seahorse Assay was performed to measure the increase in oxygen consumed due to the presentation of a long chain FA, palmitate. The MTP deficient CMs were expected to display a hindered ability to utilize long chain FAs in comparison to the WT CMs. However, it was found that all CMs, even the control CMs, were unable to utilize long chain FAs (FIG. 1F). hiPSC-CMs are immature cells representative of a FCM rather than an ACM, which is why they are unable to utilize FAs as a substrate for ATP production. Consequently, a strategy was required to mature the hiPSC-CMs so that they could utilize FAs allowing better assessment of the functional phenotype of the MTP deficient CMs.

Screening microRNAs (miRs) for hPSC-CM Maturation

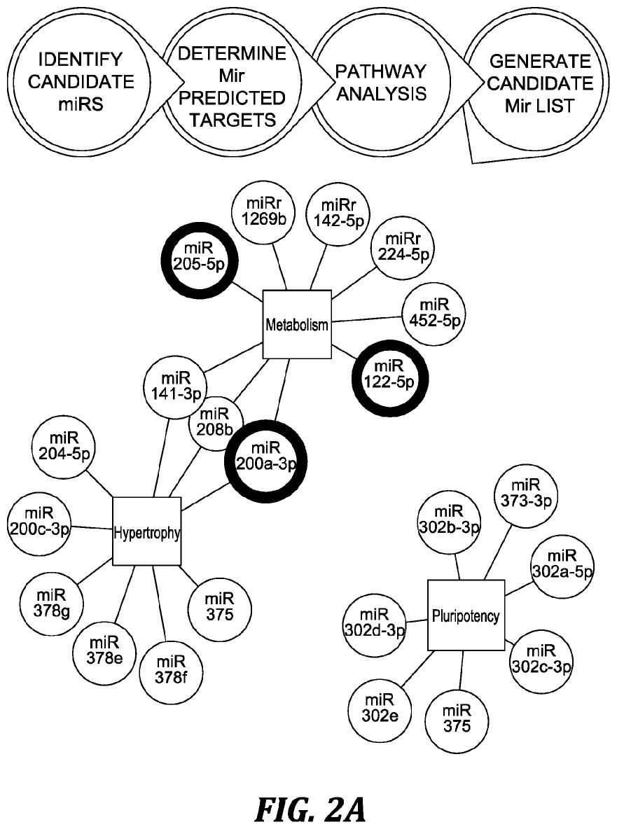

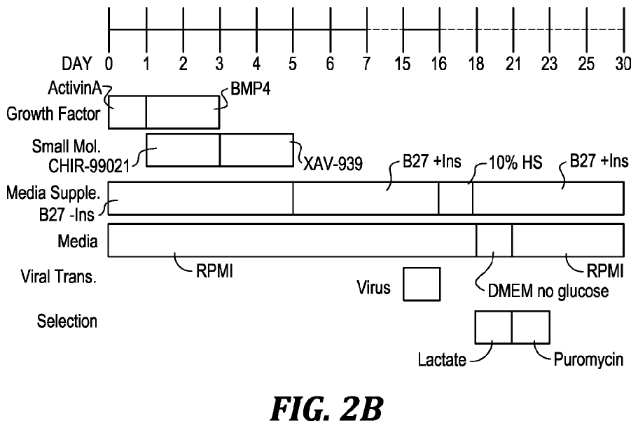

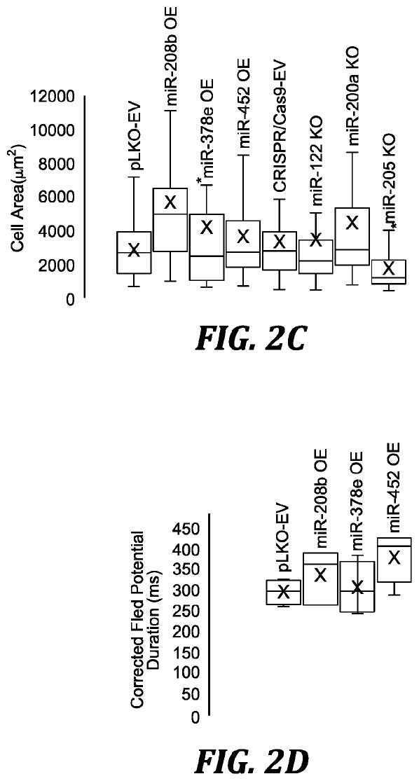

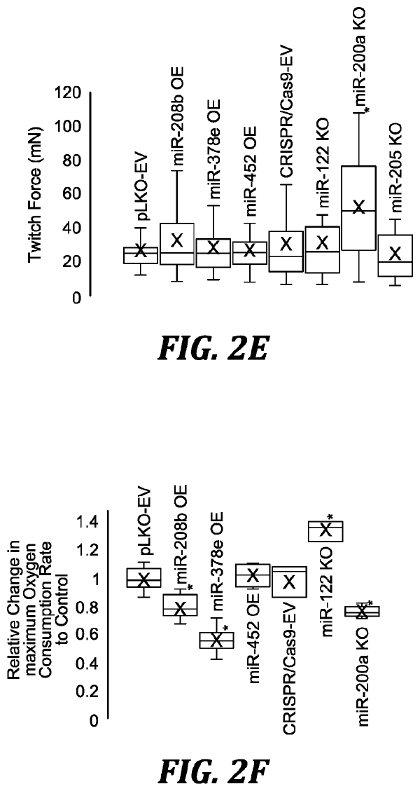

To better understand the biological changes that occur during human cardiac maturation, the inventors previously conducted a miR screen where many significantly regulated miRs were observed during the in vitro transition between Day-20 (D20) hPSC-CMs and 1-year matured hPSC-CMs [Kuppusamy, K. T., et al., Let-7 family of microRNA is required for maturation and adult-like metabolism in stem cell-derived cardiomyocytes. Proc Natl Acad Sci U.S.A., 2015]. This list was cross-referenced to in vivo miR-sequencing data of human fetal ventricular to adult ventricular myocardium [Akat, K. M., et al., Comparative RNA-sequencing analysis of myocardial and circulating small RNAs in human heart failure and their utility as biomarkers. Proc Natl Acad Sci USA, 2014. 111(30): p. 11151-6; Yang, K. C., et al., Deep RNA sequencing reveals dynamic regulation of myocardial noncoding RNAs in failing human heart and remodeling with mechanical circulatory support. Circulation, 2014. 129(9): p. 1009-21]. The inventors previously found the highest up-regulated family of miRs, Let-7, could be overexpressed in hESC-CMs to drive a robust, albeit incomplete, maturation response [Kuppusamy, K. T., et al. Proc Natl Acad Sci U.S.A., 2015]. Here, additional miRs were combined together with Let-7 to rapidly mature hPSC-CMs by promoting a more complete adult like transcriptome. The top 15 up- and down-regulated miRs were selected from the screen and the top 200 predicted targets (TargetScanHuman) were identified for each miR. Using each miR's predicted targets, pathway analysis was performed using GeneAnalytics software to determine which miRs were affecting pathways associated with cardiomyocyte maturation. These included glucose and/or fatty acid metabolism, cell growth and hypertrophy, and cell cycle. Many down-regulated miRs were associated with maintenance of a pluripotent state and were not chosen to screen for cardiomyocyte maturation. Ultimately, six miRs were chosen for further analysis, based on the pathway analysis, to assess for their CM maturation potency: three up-regulated miRs (miR-452, -208b and -378e) and three down-regulated miRs (miR-122, -200a, and -205) (FIG. 2A).

Specifically, the three candidate highly up-regulated miRs chosen were miR-378e [Nagalingam, R. S., et al., A cardiac-enriched microRNA, miR-378, blocks cardiac hypertrophy by targeting Ras signaling. The Journal of biological chemistry, 2013. 288(16): p. 11216-32], -208b [Callis, T. E., et al., MicroRNA-208a is a regulator of cardiac hypertrophy and conduction in mice. J Clin Invest, 2009. 119(9): p. 2772-86] and -452. The family of 378 miRs was chosen due to their high expression level in matured CMs and involvement in cardiac hypertrophy. MiR-378e and -378f share the same seed region and miR-378e was chosen as the representative miR for the 378 family. Mir-208b was chosen due to its predicted involvement in both metabolic and cardiac hypertrophic pathways. Furthermore, miR-208b is an intronic miR in the gene myosin β-heavy chain (MYH7) and has been reported to have roles in specifying slow muscle fibers while repressing fast muscle fiber gene programs in mouse hearts [van Rooij, E., et al., A family of microRNAs encoded by myosin genes governs myosin expression and muscle performance. Dev Cell, 2009. 17(5): p. 662-73]. MiR-452 was the second highest up-regulated miR, after Let-7, and was found to have predicted targets associated with metabolism.

The three candidate highly down-regulated miRs chosen were miR-200a, -122 and -205. MiR-141 and -200a share the same seed region and are involved in both hypertrophy and metabolism pathways. MiR-200a was chosen as the representative miR to study. The other two highly down-regulated miRs, miR-205 and miR-122, showed the greatest degree of down-regulation.

Functional Analysis of Candidate microRNAs (miRs)

The six miRs indicated above were assessed using four functional tests to determine hPSC-CM maturation: cell area, force of contraction, metabolic capacity and electrophysiology. WT D15 hiPSC-CMs were transduced with a lentivirus to either OE a miR or KO a miR using CRISPR/Cas9. Cells were then lactate-selected to enrich for the cardiomyocyte population and puromycin-selected to enrich for the population containing the viral vector. Functional assessment was performed after two weeks of miR perturbation on D30 (FIG. 2B).

An important feature of cardiomyocyte maturation is an increase in cell size. Out of the tested miRs, only miR-208b OE was found to induce a significant increase in cell area (EV: 2891 μm², 208b: 5802 μm², P<0.05) (FIG. 2C). Immature hPSC-CMs spontaneously beat at a high rate and have a short field potential duration when studied by extracellular micro-electrodes. Using micro-electrode arrays, the OE miRs were assessed for whether they increased the field potential duration to a physiologically relevant length. Out of the tested miRs, only miR-452 OE increased the corrected field potential duration (cFPD) to a more adult like duration (cFPD, EV: 296 ms, 452: 380 ms) (FIG. 2D). One of the hallmarks of cardiomyocyte maturation is the increase in contractile force generated by the cell. Single cell force of contraction analysis was performed using a micropost platform [Kuppusamy, K. T., et al., Proc Natl Acad Sci USA, 2015; Rodriguez, M. L., et al., Measuring the contractile forces of human induced pluripotent stem cell-derived cardiomyocytes with arrays of microposts. J Biomech Eng, 2014. 136(5): p. 051005; Beussman, K. M., et al., Micropost arrays for measuring stem cell-derived cardiomyocyte contractility. Methods, 2016. 94: p. 43-50]. Out of the tested miRs, only the KO of miR-200a brought about a significant increase in force of contraction (EV: 30.8 nN, miR-200a: 51.7 nN, P<0.05) (FIG. 2E). Finally, the metabolic capacity of miR treated hPSC-CMs was assessed. Cardiomyocytes are a metabolically demanding cell type necessitating mitochondria that have a high capacity for ATP synthesis. Out of the tested miRs, only the KO of miR-122 brought about a significant increase in maximum oxygen consumption rate (OCR) indicating more active mitochondria (miR-122 KO: 1.35 fold change compared to EV, P<0.001) (FIG. 2F).

Bioinformatic Analysis of Candidate microRNAs (miRs)

RNA-Sequencing was performed after alterations of some of the miRs (miR-378e OE, -208b OE, -452 OE, -122 KO or -205 KO) to assess their global transcriptional impact in hPSC-CMs. To determine if each miR was able to generate a differential effect on a global transcript level the samples were analysed using principal component analysis (PCA). In each sample, approximately 11,000 protein-coding genes were expressed with an aggregated expression of at least three FPKM across all samples were used for PCA. PCA showed that each miR was able to bring a significant change from their respective controls (not shown). MiR-452 OE had the largest separation on PC1 while miR-122 KO had the largest separation on PC2. This suggests that each of these two miRs have a robust influence on the hPSC-CM transcriptional profile. Furthermore, since none of the miRs clustered with one another, each miR was capable of inducing a unique expression signature.

Each miR's function was then analyzed in a more targeted manner by specifically examining pathways that are essential for cardiac maturation. A pathway enrichment heat map was generated showing how each miR influenced seven different pathways chosen as hallmarks of cardiomyocyte maturation (characterized as cardiac hypertrophy, cardiac identity, cell cycle, electrophysiology, fatty acid metabolism, glucose metabolism, and cytoskeleton; not shown). MiR-122 KO had an up-regulation of cell cycle and fatty acid metabolism genes. MiR-452 OE showed an up-regulation of cardiac hypertrophy, electrophysiology and cytoskeleton. MiR-208b OE showed a strong up-regulation of cardiac identity along with cell cycle and electrophysiology genes. Finally, miR-378e OE showed an up-regulation of electrophysiology genes while miR-205 KO showed poor up-regulation of cardiac maturation related pathways. This heat map reinforces that each miR has a unique influence on cardiomyocyte maturation, as each miR brought about a different set of pathway enrichment. Furthermore, based on the heat map data miR-205 KO had a poor ability to bring about cardiomyocyte maturation while miRs-122 KO, -452 OE and -208b OE all showed a strong ability to influence hallmark pathways of cardiomyocyte maturation.

From these data, a MicroRNA Maturation Cocktail was generated, termed MiMaC, that included constructs for: Let7i OE, miR-452 OE, miR-122 KO, and miR-200a KO. Let7i was chosen due to the inventors' initial study showing the potency of this miR to bring about cardiomyocyte maturation [Kuppusamy, K. T., et al., Proc Natl Acad Sci USA, 2015]. From each of the functional assays, a miR was chosen that brought a significant increase in maturation to generate a cocktail that consisted of the smallest number of miRs.

Functional Assessment of MiMaC

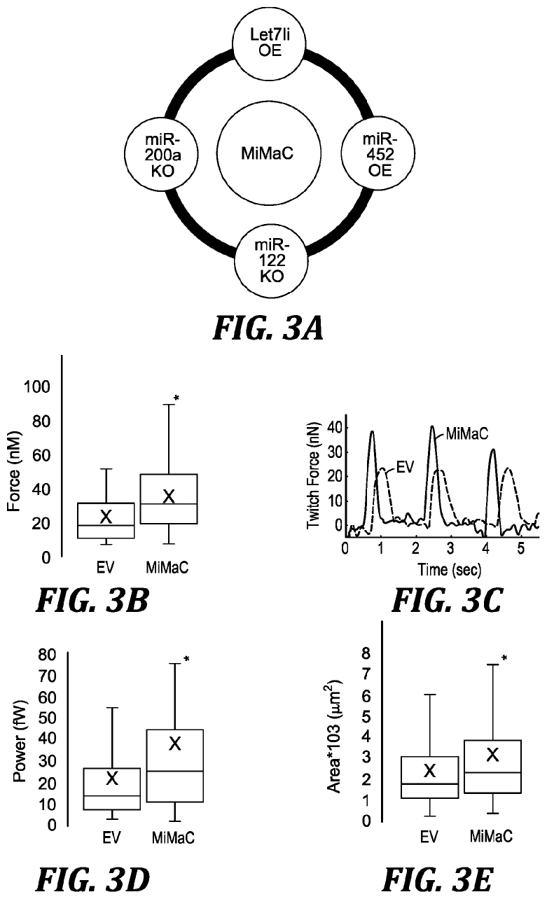

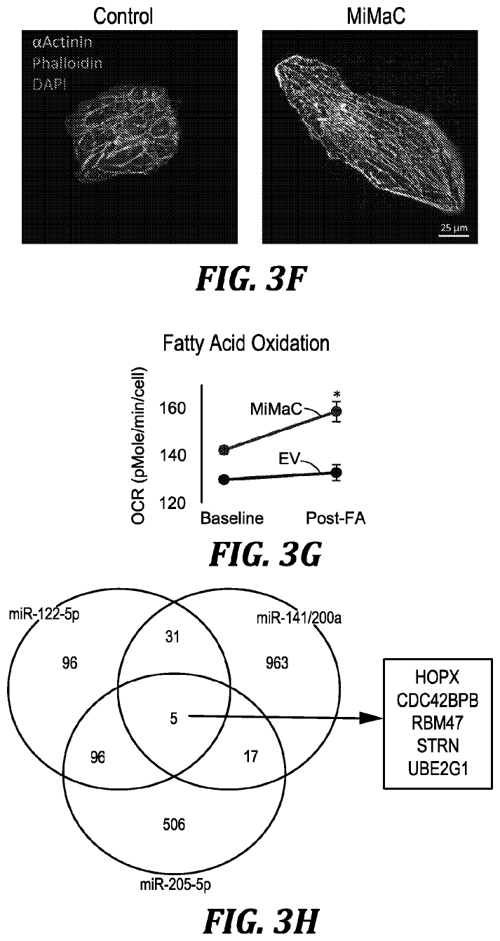

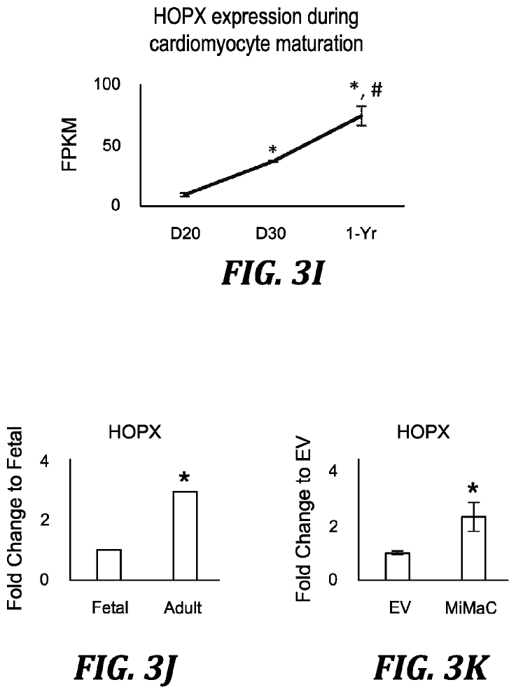

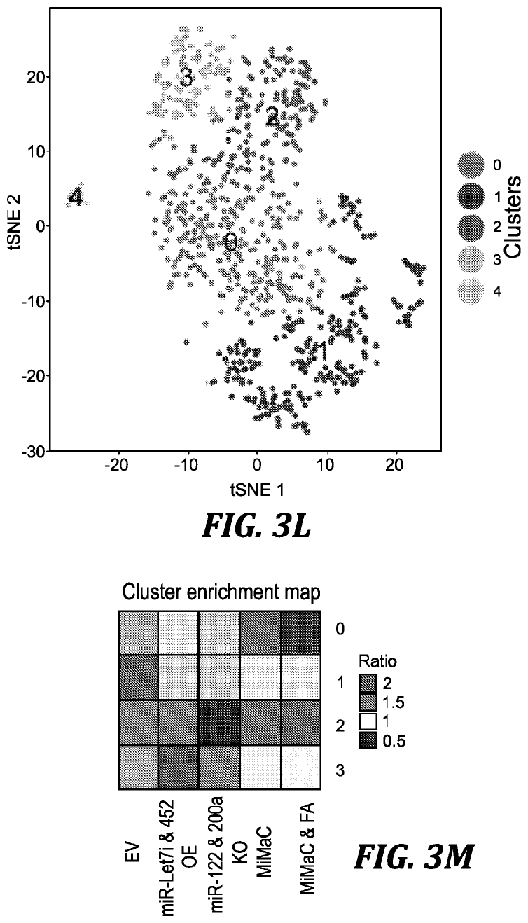

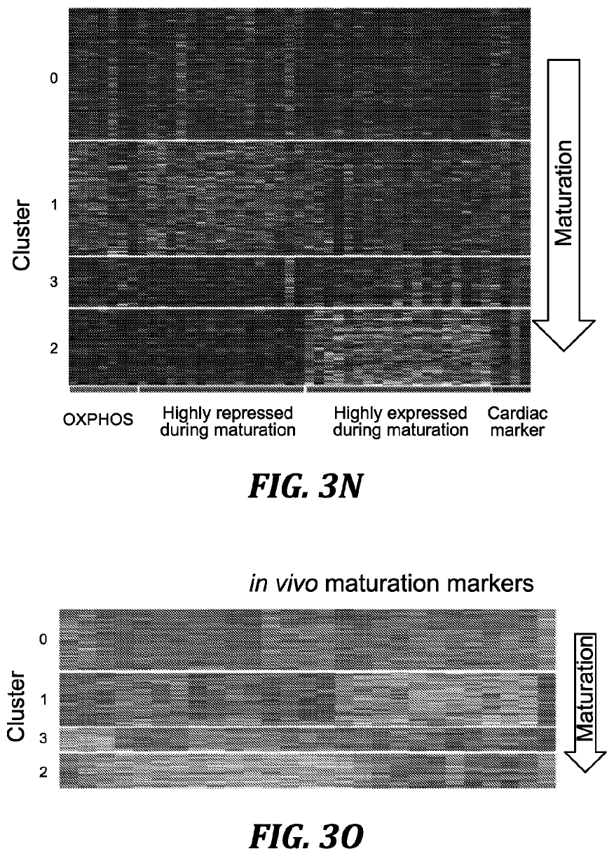

To assess MiMaC treated hPSC-CM maturation we performed force of contraction, cell area and metabolic assays (FIG. 3A). MiMaC treated hiPSC-CMs had a statistically significant increase in twitch force (mean force: 36 nN, P=0.002) as compared to control cells (mean force: 24 nN) (FIG. 3B). MiMaC treated hiPSC-CMs also had a statistically significant increase in power generated (mean power: 38 fW, P=0.016) as compared to control cells (mean power: 22 fW) (FIG. 3D).

MiMaC treated hPSC-CMs had a statistically significant increase in cell area. Using hiPSC-CMs, MiMaC treated CMs had a mean area of 3022 μm², P<0.001, as compared to control cells which had a mean area of 2389 μm² (FIGS. 3E and 3F). In addition to MiMaC's effect on hiPSC-CMs, MiMaC also significantly increased the cell area of treated hESC-CMs (not shown).

One of the hallmarks of cardiomyocyte maturation is gaining the ability to utilize FAs to generate ATP. Immature hPSC-CMs are unable to utilize long-chain FAs for ATP production via β-oxidation. To assess whether MiMaC treated hPSC-CMs could oxidize long-chain FAs, the cells were acutely challenged with palmitate and measured if there was an increase in OCR. Both MiMaC-treated hESC-CMs and hiPSC-CMs were able to utilize palmitate significantly greater than control CMs (see FIG. 3G addressing hiPSC-CMs; similar results for hESC-CMs not shown).

Transcriptional Assessment of MiMaC

To gain a better understanding of how MiMaC was affecting the transcriptome of hiPSC-CMs, RNA-Sequencing was performed comparing D30 EV control CMs to D30 MiMaC treated CMs. Pathway enrichment analysis using a hallmark gene set showed that many cell maturation and muscle processes were up-regulated such as: myogenesis and epithelial mesenchymal transition [34]. The top down-regulated pathways were associated with cell cycle, a key feature of cardiomyocyte maturation. Using STRING Analysis, we determined the network of significantly up-regulated and interconnected genes associated with two pathways: myogenesis and epithelial mesenchymal transition. STRING Analysis was also used to show that the significantly down-regulated and interconnected genes associated with repressed cell cycle were the mitotic spindle and G2M checkpoint. These findings demonstrate that the MiMaC tool promotes a more mature transcriptome in hiPSC-CMs.

HOPX is a Novel Regulator of CM Maturation

To better understand the molecular mechanisms that are critical for cardiac maturation, the overlapping predicted targets of the chosen six miRs were determined. One of the predicted targets, HOPX (FIG. 3H), is important for cardiomyoblast specification [Jain, R., et al., HEART DEVELOPMENT Integration of Bmp and Wnt signaling by Hopx specifies commitment of cardiomyoblasts. Science, 2015. 348(6242): p. aaa6071], yet no work on this transcriptional regulator has addressed the later process, human cardiomyocyte maturation. Here, it was determined that HOPX expression was up-regulated in vitro (FIG. 3I), in vivo (FIG. 3J) and in MiMaC treated hiPSC-CMs (FIG. 3K). To analyze how the selected MiMaC miRs might individually regulate HOPX expression during maturation, HOPX levels were analyzed in miR-122 KO and Let7i OE hiPSC-CMs. HOPX was found to be up-regulated 6.8 fold in D30 miR-122 KO hiPSC-CMs while Let7i OE matured hiPSC-CMs had no effect on HOPX expression (not shown). These data indicate that Let7i OE maturation does not govern HOPX cardiac maturation pathways. This highlights the necessity of combining multiple miRs together to generate a robust maturation effect in hPSC-CMs and that HOPX seems to be a strong candidate for post-committed cardiomyocyte maturation.

scRNA-Sequencing Analysis of miR Treated CM Maturation

Using single cell RNA-Sequencing (scRNA-Seq), the MiMaC tool was utilized to provide further insight into the underlying mechanisms of cardiomyocyte maturation and to garner a better understanding of how each miR that constitutes MiMaC behaves in CM maturation. scRNA-Seq was performed on five groups of miR treated CMs: EV, Let7i & miR-452 OE, miR-122 & -200a KO, MiMaC and MiMaC+FA. Unbiased clustering was performed to determine how the miR perturbations changed CMs; five subgroups were discovered (FIG. 3L) and used the Chi-square test to assess whether the miR perturbations resulted in enrichments in these five clusters (FIG. 3M). The EV group was enriched in clusters 0 and 3, Let7i and miR-452 OE group was enriched in clusters 0 and 1, miR-122 and -200a KO group was enriched in clusters 0 and 3 and MiMaC and MiMaC+FA were enriched in clusters 1 and 2. Cluster 4 mainly consisted of cells with poor read counts and was not analyzed further. Characterizing the cell fate in each subgroup showed the majority of cells were cardiomyocytes with a very small subset of cells in cluster 1 displaying fibroblast (ENC1, DCN and THY1) and epicardial markers (WT1, TBX18) (not shown). These data indicate that the lactate enrichment protocol successfully generated highly enriched cardiomyocyte populations.

To rank which clusters had a higher degree of cardiomyocyte maturation, the scRNA-Seq clusters were assessed in two different ways. First, the genes highly up- and down-regulated in the MiMaC enriched cluster, cluster 2, were assessed along with cardiac markers and oxidative phosphorylation genes (FIG. 3N). Next, in vivo human cardiac maturation markers in the identified clusters were examined (FIG. 3O). Cluster 2 was found to have genes associated with myofibril structural proteins highly up-regulated and ribosomal and ECM adhesion genes down-regulated (FIG. 3N). The mean expression levels of the in vivo maturation marker genes were significantly higher in cluster 2 as compared to the other clusters (FIG. 3O; P<2×10¹⁶, using linear mixed effects model). These same analyses were also performed based on the experimental groups. The MiMaC treated cells were also found to be the most mature as demonstrated in a series of tSNE plots and heatmaps (not shown). Based on these findings, each cluster was ranked from least mature to most mature as cluster: 0<1<3<2. Cluster 2, the most mature CM cluster enriched for the MiMaC treated CMs, showed the highest expression of HOPX, a gene that is up-regulated in maturation and is the predicted target of the down-regulated miRs in MiMaC (not shown). Importantly, these data indicate that the observed transcriptional maturation mirrors normal in vivo cardiomyocyte maturation (FIG. 3O).

Finally, the addition of fatty acids to the MiMaC formulation was assessed to increase cardiomyocyte maturation. Three long chain fatty acids, palmitate, linoleic and oleic acid were added to the basal cardiac media used. We found the MiMaC+FA cells were enriched in cluster 2. While some studies have shown lipotoxicity with particular FAs, the analysis of the carefully optimized FA-treatment procedure showed no increase in transcripts indicative of apoptosis, indicating minimal lipotoxicity in this assay (not shown). These data indicate that MiMaC was essential to bring about a robust transcriptional maturation of our hiPSC-CMs and that it was necessary to incorporate all four microRNAs together to bring about this robust maturation response.

scRNA-Seq Reveals an Intermediate Cardiomyocyte Maturation Stage

After unbiased analysis of the miR treated CMs it was clear each miR combination resulted in enrichment of different states of CM maturation. Interestingly, cardiomyocyte cluster 1, enriched for Let7i and miR-452 OE, showed a robust up-regulation of OXPHOS and Myc target genes but was not yet significantly increased in most cardiomyocyte maturation markers (FIGS. 3N and 3O). Hence, treatment of Let7i and miR-452 OE created an intermediate maturity CM in which the metabolic maturation was the leading force. These data suggest a possible intermediate stage is a necessary transition stage between a fetal like CM to a more mature CM which requires transient up-regulation of OXPHOS genes.

MTP/HADHA Deficient CMs Display Reduced Mitochondrial Function

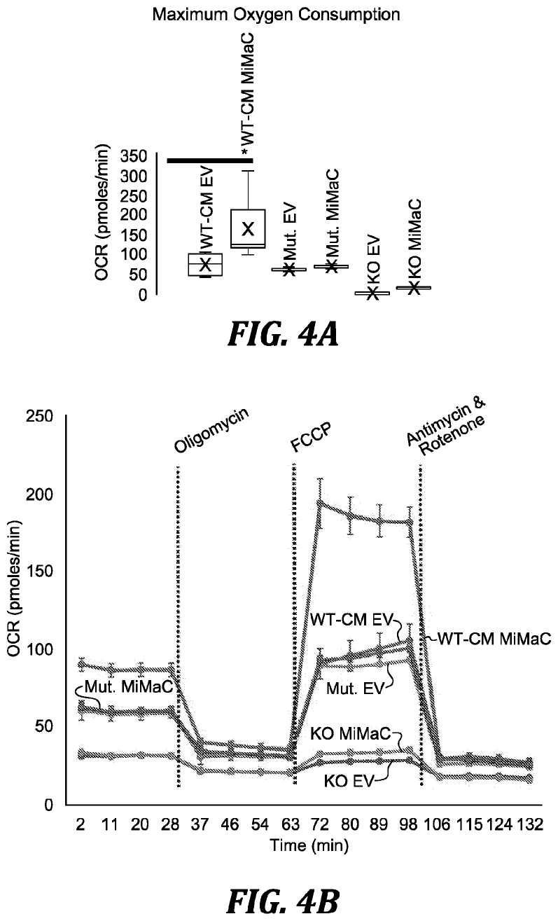

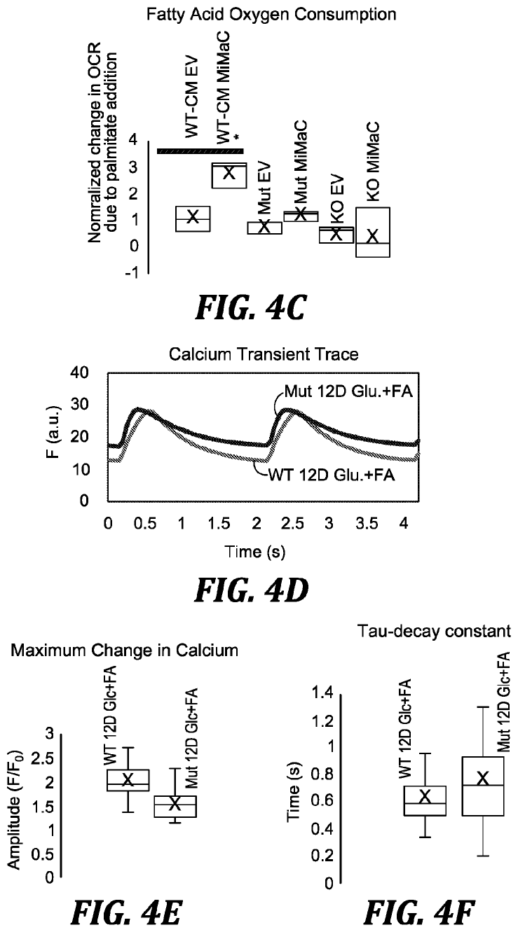

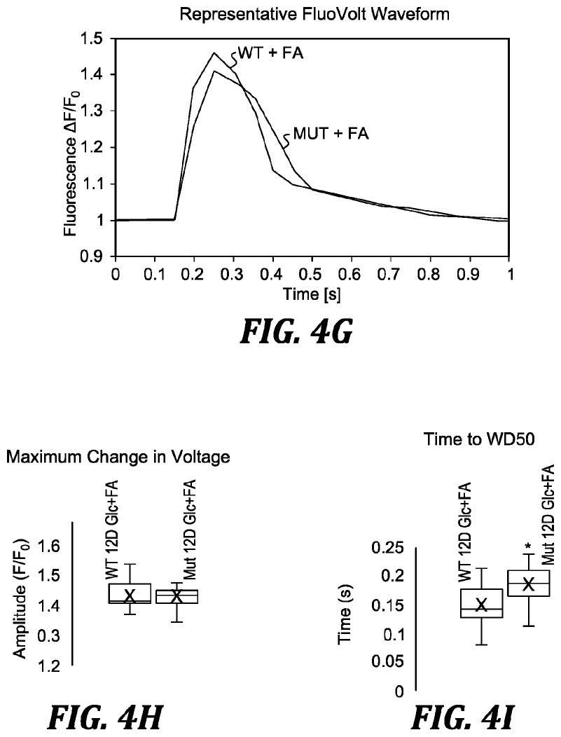

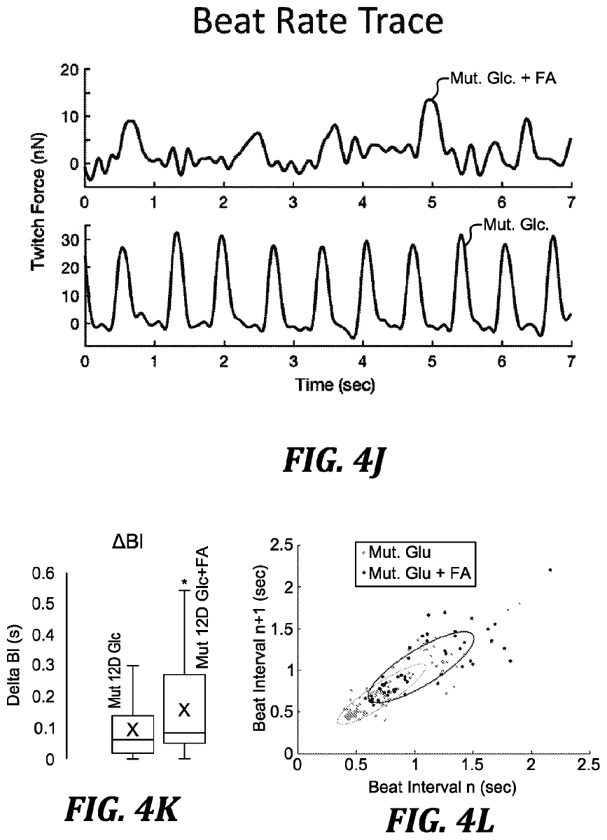

The generation of the MiMaC tool allowed the study HADHA CM disease etiology. Because immature hPSC-CMs were unable to oxidize fatty acids, it was necessary to mature the HADHA Mut and KO CMs with MiMaC, which brings about fatty acid oxidation in WT CMs. First, the maximum OCR of WT, HADHA Mut and KO CMs was assessed. MiMaC treated WT CMs had a statistically significant increase in maximum OCR (2.2 fold change) as compared to control cells (FIGS. 4A and 4B). Interestingly, control and MiMaC treated HADHA Mut CMs had maximum OCR similar to control WT-CMs while the HADHA KO CMs had depressed maximum OCR. These data suggest defective mitochondrial activity of HADHA in Mut and KO CMs.

Next, it was assessed whether MiMaC treated HADHA Mut and KO CMs could utilize the fatty acid palmitate for ATP production. Only MiMaC-treated WT CMs showed a statistically significant increase in oxygen consumption due to palmitate addition (FIG. 4C). WT control CMs along with control and MiMaC treated HADHA Mut and KO CMs were unable to utilize FAs. These data show that MiMaC treated CMs have the capacity to utilize long chain FAs. However, MiMaC treated HADHA Mut and KO CMs are unable to do so. MiMaC was essential to assess the FAO limitations of the HADHA Mut and KO CMs.

Abnormal Calcium Handling of HADHA Mut CMs

MTP deficient infants can present with sudden, initially unexplained death after birth. It is proposed that the stress of lipids, the main substrate for ATP production found in a mother's breast-milk, is what precipitates the early infant death due to MTP deficiency. To address this hypothesis, a combination of three long chain fatty acids supplemented to the base cardiac media which contains glucose (Glc+FA media): palmitate, oleic and linoleic acid, because these FAs are the most abundant in the serum of breastfed human infants. Palmitate, as a fatty acid substrate, is one of the most abundant fatty acids circulating during the neonatal period, representing 36% of all long-chain free fatty acids. While challenging CMs with FAs can lead to lipotoxicity, a concentration and combination of three fatty acids that do not result in lipotoxicity was carefully developed (FIG. 3L).

To better understand the way in which MTP deficient CMs may be precipitating an arrhythmic state leading to SIDS, calcium transients were measured in the WT and HADHA Mut CMs (see, e.g., FIG. 4D). The fold change in calcium being cycled was found to be significantly higher in WT CMs as compared to HADHA Mut CMs (WT CM: 2.03, Mut CM: 1.55, P<0.001) (FIG. 4E) with no change in calcium rise velocity (not shown). This suggested calcium was being cycled from the cytosol and stored in an aberrant manner in HADHA Mut CMs. When examining the tau-decay constant, HADHA Mut CMs were found to have a higher average value (WT CM: 0.63 s, Mut CM: 0.76 s) (FIG. 4F). This suggested the rate at which calcium was being pumped back into the sarco/endoplasmic reticulum was slower in the HADHA Mut CMs.

Delayed Repolarization and Beat Rate Abnormalities in HADHA Mut CMs

Because HADHA Mut CMs cultured in Glc+FA media exhibited abnormal calcium cycling, it was assessed whether or not these CMs also exhibited abnormal electrophysiology. It was determined membrane potential changes using a voltage sensitive fluorescent dye, Fluovolt. It was found that while HADHA Mut CMs had no change in the maximum change in voltage amplitude, the time to reach maximum depolarization, or the rate of depolarization (FIGS. 4G and 4H), significant differences were observed when examining repolarization rates. The time to wave duration (WD) 50% (WD50) and 90% (WD90) were found to be significantly longer in the HADHA Mut CMs as compared to WT CMs (see FIG. 4I for the WD50; similar results were observed for WD90, not shown). These data indicate that the HAHDA Mut CMs had impaired repolarization. This phenotype can be caused by the observed abnormal calcium dynamics due to impaired cycling of calcium back into the sarcoendoplasmic reticulum.

Because HADHA Mut CMs exhibited defective calcium handling and electrophysiology, it was assessed whether these CMs exhibited beat rate abnormalities. The spontaneous beating of HADHA Mut CMs was tracked in the presence of FAs to quantify beat rate abnormalities. HADHA Mut CMs displayed abnormal beat rate variability as the time between beats was not even (see, e.g., FIG. 4J). Quantifying these findings, it was found that the HADHA Mut CMs had a significantly higher beat interval (not shown) and a significantly higher change in beat-to-beat interval (ABI) (FIG. 4K). These data indicate that HADHA Mut CMs beat on average slower and the time between beats was more variable. Furthermore, the percentage of ΔBI that were greater than 250 ms were quantified and on average the HADHA Mut CMs after 12D of Glc+FA media had a higher percentage of potentially arrhythmic ΔBIs (˜30%) compared to Mut CMs in Glc media (˜10%). This quantification of the number of cells with a ABI greater than 250 ms suggested erratically beating cells. Finally, a Poincaré plot was generated with fitted ellipses (95% confidence interval) around each group's beat interval data (FIG. 4L). A narrow and elongated ellipse suggested uniform beat intervals while a more rounded ellipse suggested beat rate abnormalities. Taking the ratio of the major to minor axis of each ellipse we found that the HADHA Mut Glc condition had a ratio of 4.36 while the HADHA Mut Glc+FA condition had a ratio of 3.12 indicating that the HADHA Mut Glc+FA condition had a more rounded ellipse meaning more beat-to-beat variability in these CMs.

Single Cell RNA-Sequencing Identifies HADHA Mut CM Subpopulations

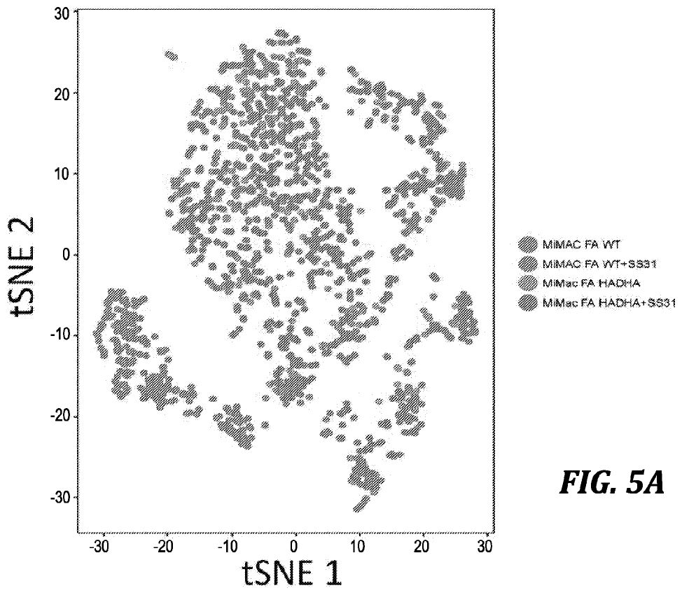

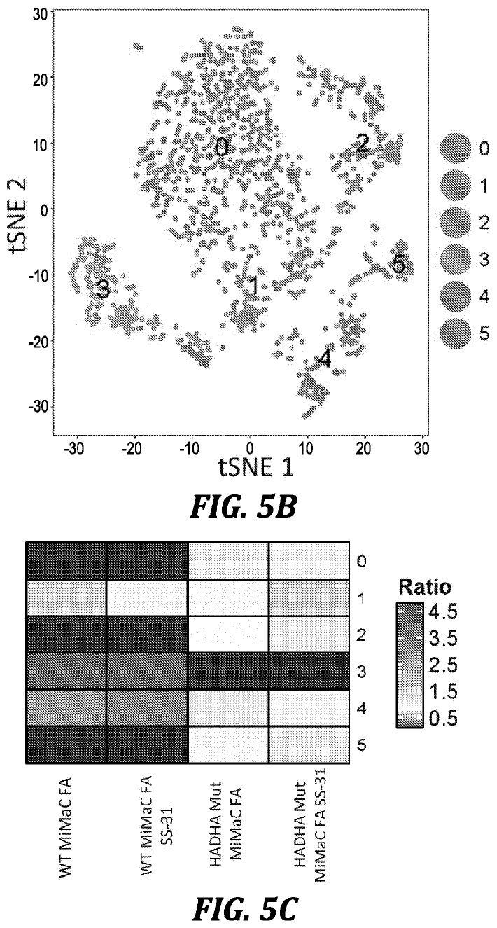

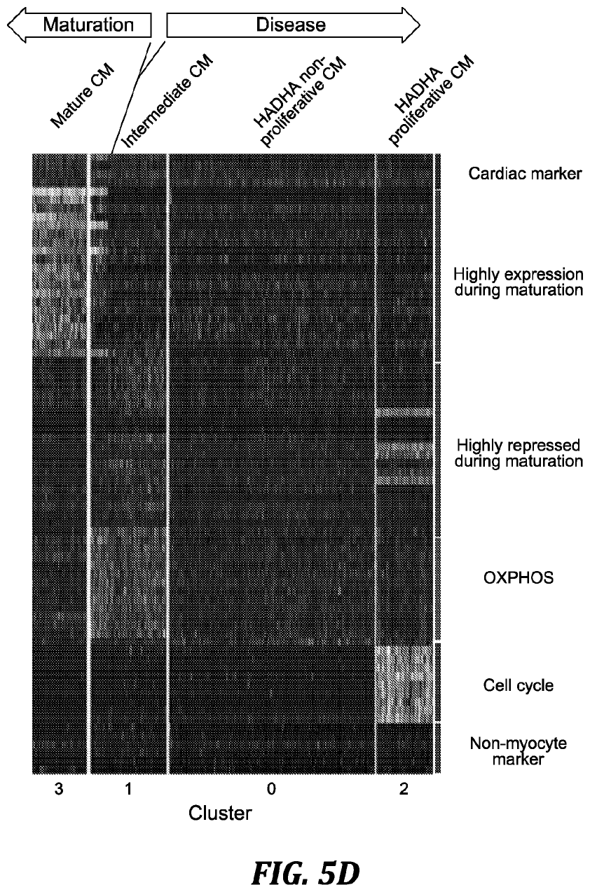

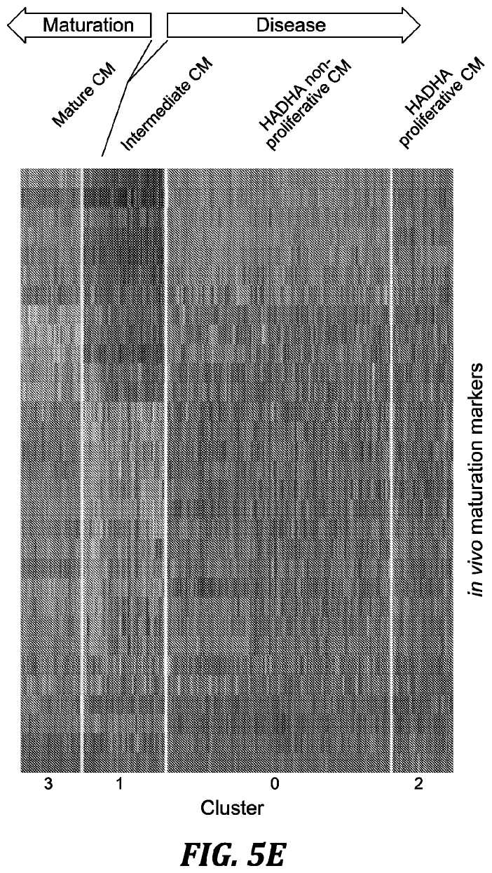

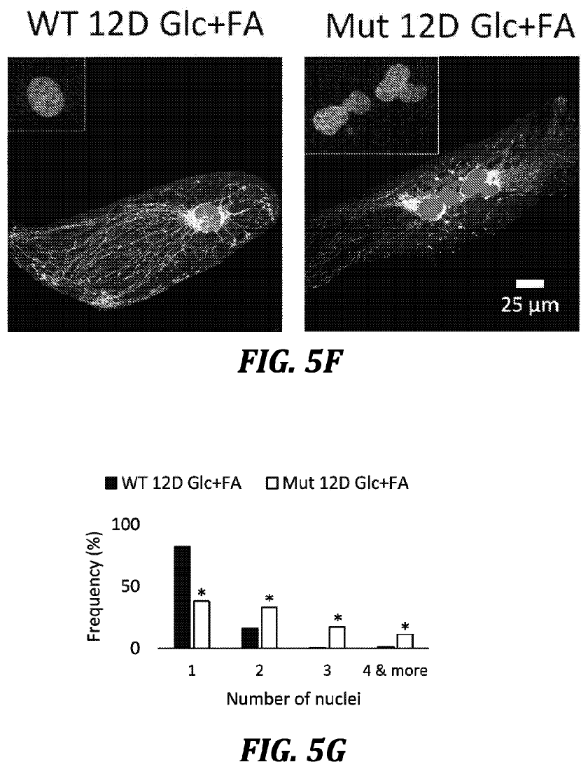

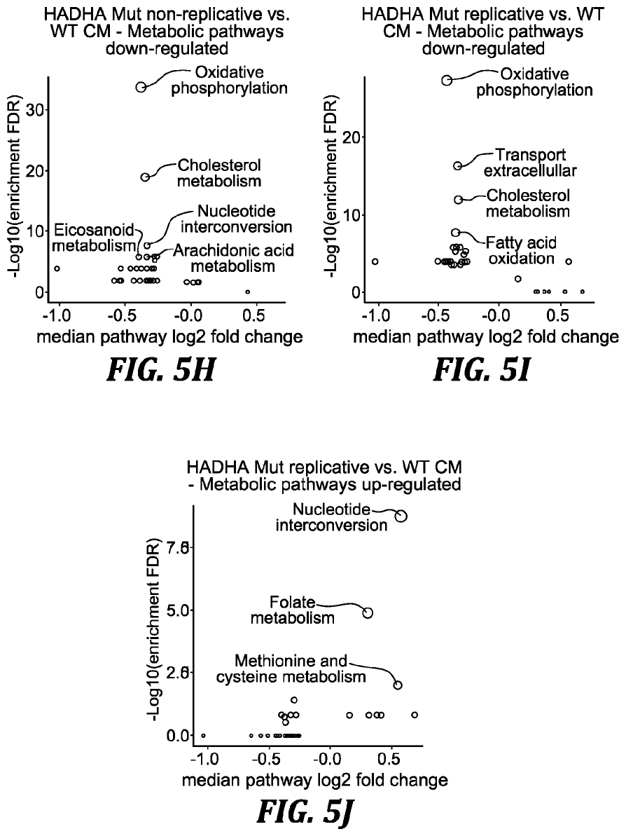

Single cell RNA-Sequencing was performed to better understand how the HADHA Mut CM population was behaving when challenged with FAs. A tSNE plot detailing each of the sequenced cell groups showed a clear distinction between WT and HADHA Mut CMs, with a small but significant overlap (FIG. 5A). When performing unbiased clustering, 6 clusters were found: 0 HADHA Mut CMs non-replicating, 1 an intermediate maturation population of WT and Mut CMs, 2 HADHA Mut CMs replicating, 3 healthy CMs, 4 fibroblast like population, 5 epicardial like population (FIGS. 5B and 5C).

To assess the degree of maturation and disease state, each cluster was categorized based on the key categories described above (FIG. 3N). Up-regulated genes in cluster 3 were associated with myofibril assembly and striated muscle cell development while down-regulated genes in cluster 3 were associated with ribosomal proteins and ECM associated proteins. Interestingly, a subset of both WT and HADHA Mut CMs were identified in an intermediate CM maturation cluster, cluster 1, as described above (FIGS. 3L and 5D). This cardiac population had a high up-regulation of OXPHOS and Myc target genes such as FABP3, COX6C, ATPSE, UQZRQ, NDUFA1, and COX7B. WT cells that further developed from this intermediate state were identified in the more mature CM state, cluster 3. HADHA Mut cells, however, entered two different pathological states of disease. It was postulated that first, HADHA Mut cells lose many highly expressed and repressed cardiac markers along with cell cycle inhibitor CDKN1A, as seen in cluster 0 (Supplemental FIG. 5C). Finally, very diseased HADHA Mut CMs in cluster 2 up-regulate genes that should be highly repressed in mature CMs, and activate cell cycle genes (FIG. 5D). For example, tSNE plots demonstrated HADHA Mut CMs lose cell cycle repressor CDKN1A and a subset of HADHA Mut CMs gain markers for proliferation: MKI67 and RRM2, (not shown). These stages of maturation and disease progression were benchmarked against in vivo mouse and human maturation markers and a similar trend was found for maturation, disease progression and loss of cardiac identity (FIG. 5E).

Examining significantly changed hallmark pathways between HADHA Mut CM clusters and the WT CM cluster, OXHPOS, cardiac processes and myogenesis were found to be depressed in the mutant cells. Furthermore, while WT CMs show strong expression of cell cycle repressor CDKN1A, both HADHA Mut CM populations lost this expression. Cluster 2, the replicating HADHA Mut CMs, had an up-regulation of DNA replication, G2M checkpoint and mitotic spindle genes. Moreover, genes that are expressed in replicating and/or endocycling cells such as MKI67 and RRM2 were expressed only in cluster 2 HADHA Mut CMs. To address potential pathological outcomes of the abnormal cell cycle marker increase, the number of nuclei per cell in HADHA mutant CM were analyzed. Importantly, we observed a significant increase of the nuclei per cell in HAHDA Mut CMs as compared to WT CMs (Chi square test P<0.001) (FIGS. 5F and 5G). The majority of WT CMs were mono- or bi-nucleated, which is the healthy state found in vivo for nuclei number in CMs. However, the number of mono-nucleated HADHA Mut CMs was significantly reduced while the number of bi- and multi-nucleated HADHA Mut CMs were increased suggesting a pathological state in the HADHA Mut CMs. These data support the surprisingly high cell cycle transcript expression demonstrated in a subpopulation of HADHA Mut CMs (cluster 2). These data suggest multiple stages of disease state in the HADHA mutant CMs.

To ensure cell cycle was not the underlying difference between all clusters, cell cycle genes were examined in each cluster. Unlike previous studies which found that the bias imposed on cluster differences was dictated by which state of the cell cycle the cells were in, it was found that only cluster 2 (FIG. 5B) showed up-regulated cell cycle genes. The clustering data was also re-processed with the removal of cell cycle genes and all clusters remained, except for original cluster 2, high cell cycle HADHA Mut CMs. These findings suggest that cell cycle is the underlying reason for cluster 2 but not for the rest of the cell populations (FIGS. 5A and 5B).

Based on this data, three different states of pathology were postulated in HADHA Mut CMs challenged with FAs: intermediate state::non-replicating CM state::replicating CM state. Cluster 1 showed an intermediate state of CM maturity, characterized by elevated OXPHOS and Myc target genes. Importantly, both WT and HADHA CMs are found in cluster 1, suggesting that the HADHA CMs only manifest pathological phenotypes that separate them from wild type cells later in development, during the maturation process, similar to that seen in human development. However, cluster 0 only contained HADHA mutant CMs and showed a pathological state with depressed cell cycle repressors along with depressed metabolic and cardiac structural genes. Finally, cluster 2 was the most pathological having repressed metabolic and cardiac genes and upregulated cell cycle genes.

Unbiased metabolic pathway analysis was performed, screening 68 metabolic pathways and found HADHA Mut CM clusters, 0 and 2, displayed reduced metabolic pathway gene expression in comparison to WT CM, cluster 3 (FIGS. 5H and 5I). Specifically, OXPHOS was one of the most down-regulated pathways followed by cholesterol metabolism and fatty acid oxidation. Interestingly, in cluster 2, there were two highly up-regulated metabolic pathways: nucleotide interconversion and folate metabolism, two key metabolic processes involved in DNA synthesis (FIG. 5J). Since HADHA Mut CMs displayed a down-regulation of many metabolic pathways including fatty acid and OXPHOS genes, the mitochondria and myofibrils of these cells were then examined.

Sarcomere Degradation and Aberrant Mitochondrial Activity of HADHA Mut and KO CMs

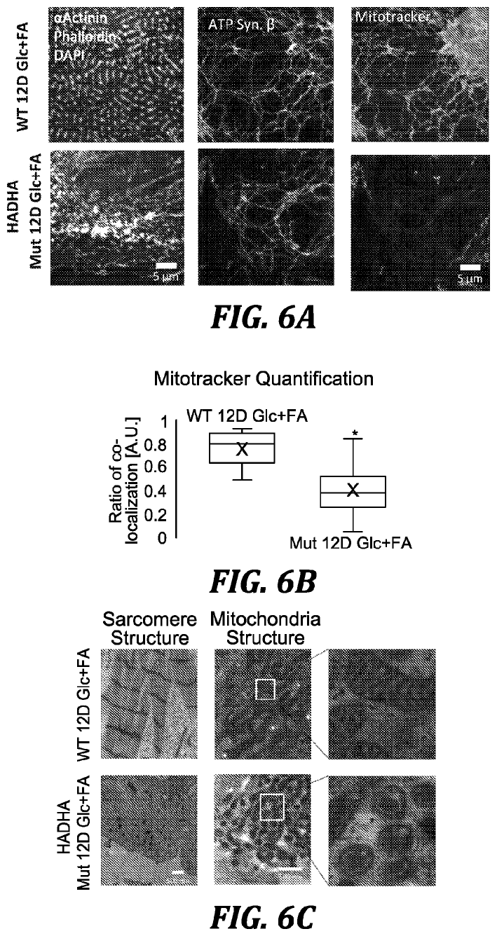

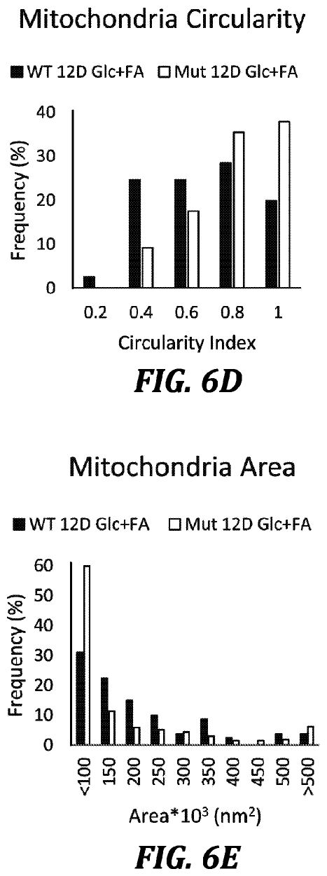

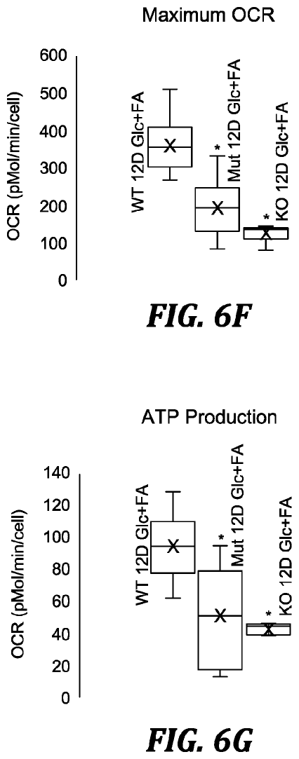

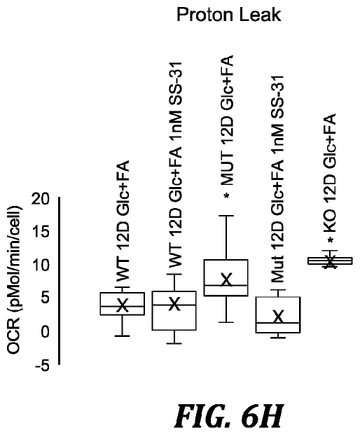

When HADHA Mut and KO CMs were cultured in glucose-media alone, no obvious defects were observed in HADHA Mut and KO compared to the WT CMs (not shown; confocal images were taken of D24 and D30 WT, HADHA Mut and HADHA KO hiPSC-CMs were cultured in glucose media, and myofibril staining of αActinin and actin (phalloidin) showed no abnormalities; mitochondrial staining with ATP synthase β subunit and mitochondrial potential gradient shown via mitotracker staining showed no mitochondrial abnormalities). However, when cultured 6-12 days in FA media, sarcomere and mitochondrial defects manifested in the HADHA Mut and KO CMs, while the WT CMs appeared normal (FIG. 6A; not shown: after 6D of glucose and fatty acid media treatment HADHA Mut and KO hiPSC-CMs displayed signs of sarcomere disruption as seen in the less defined αActinin staining and the beginnings of loss of mitochondrial proton gradient as seen from the mitotracker staining in the mutant; ATP synthase I subunit showed normal mitochondrial networks in both the WT and HADHA Mut hiPSC-CMs while there are the beginnings of loss of mitochondrial network in the HADHA KO hiPSC-CMs). After 12D of Glc+FA media treatment, WT CMs had healthy myofibrils while the HADHA Mut CMs showed sarcomere dissolution, as α-actinin staining became punctate and actin filaments were difficult to detect (FIG. 6A). Mitochondrial health was assessed next as the HADHA Mut and KO CMs were unable to process long-chain FAs. Mitochondria were stained using ATP synthase beta subunit to assess the presence of a mitochondrial network. Both the WT and HADHA Mut CMs had many connected mitochondria while the KO CMs, at 6D FA, had lost their mitochondrial network to small, more circular mitochondria. To assess the functionality of these mitochondria, the mitochondrial proton gradient was analyzed via mitotracker orange staining. After 12-days of Glc+FA rich media, HADHA Mut CMs had highly depressed mitochondrial membrane proton gradient (FIGS. 6A and 6B).

To better assess the sarcomere and mitochondrial disease phenotype, transmission electron microscopy (TEM) was performed on WT and HADHA Mut CMs after 12D of Glc+FA exposure (FIG. 6C). WT CMs showed abundant myofibrils, clear Z bands but indistinct A-bands and I-bands, and no M-lines, indicating an intermediate, normal stage of CM myofibrillogenesis. Furthermore, WT CMs showed healthy mitochondria with good cristae formation. In contrast, HADHA Mut CMs showed poor myofibrils with a disruption of Z-disk structure replaced by punctate Z-bodies and disassembled myofilaments in the cytoplasm. Interestingly, HADHA Mut CM mitochondria were small and swollen with very rudimentary cristae morphology (FIG. 6C). Quantifying the WT and HADHA Mut CM mitochondria revealed HADHA Mut mitochondria were smaller in area and more rounded as compared to WT mitochondria (FIGS. 6D and 6E). Finally, Western blot analysis examining complex I-V proteins showed that HADHA Mut CMs had depressed complex I-IV protein expression in Glc+FA conditions (not shown). These data show HADHA CMs lose sarcomere structure and mitochondrial membrane potential and morphology when exposed to FAs.

SS-31 Rescues Aberrant Proton Leak in HADHA Mut CMs Chronically Exposed to FAs

To better understand the pathological state of HADHA Mut and KO CMs exposed to chronic FAs, their mitochondria were functionally assessed. The maximum OCR of Glc+FA treated HADHA Mut and KO CMs were significantly depressed as compared to WT cells (Mut CM: 190 pmoles/min/cell, KO CM: 125 pmoles/min/cell, WT CM: 359 pmoles/min/cell, P<0.05) (FIG. 6F). Furthermore, HADHA Mut CMs displayed reduced oxygen dependent ATP production (Mut CM: 51 pmoles/min/cell, KO CM: 43 pmoles/min/cell, WT CM: 93 pmoles/min/cell, P<0.05) (FIG. 6G) and HADHA Mut CMs displayed a reduced glycolytic capacity (Mut CM: 14 mpH/min/cell, KO CM: 18 mpH/min/cell, WT CM: 23 mpH/min/cell, Mut Vs WT P<0.05) (not shown: observed via mitostress assay, and calculated as the difference between the extracellular acidification rate after oligomycin and 2-deoxy-D-glucose). Because exposure to FAs led to a reduction in mitochondrial membrane potential and reduced ATP production, it was postulated that this may be due in part to an increased proton leak. By testing the difference in OCR between repressing ATP synthase (oligomycin treatment) and repressing the electron transport chain (antimycin, rotenone), it was demonstrated that HADHA Mut and KO CMs had a significantly higher proton leak than WT CMs (Mut CM: 7.66 pmoles/min/cell, KO CM: 10.52 pmoles/min/cell, WT CM: 3.64 pmoles/min/cell, P<0.05). Previous studies revealed that elamipretide (SS-31), a mitochondrial-targeted peptide, can prevent mitochondrial depolarization and proton leak. Interestingly, a 1 nM treatment of HADHA Mut cardiomyocytes with elamipretide (SS-31) rescued the increased proton leak in Glc+FA challenged Mut CMs (FIG. 6H). These data suggest that HADHA Mut and KO CMs exposed to FAs resulted in reduced mitochondrial capacity due in part to increased proton leak.

Loss of HADHA Function Leads to Long-Chain Fatty Acid Accumulation

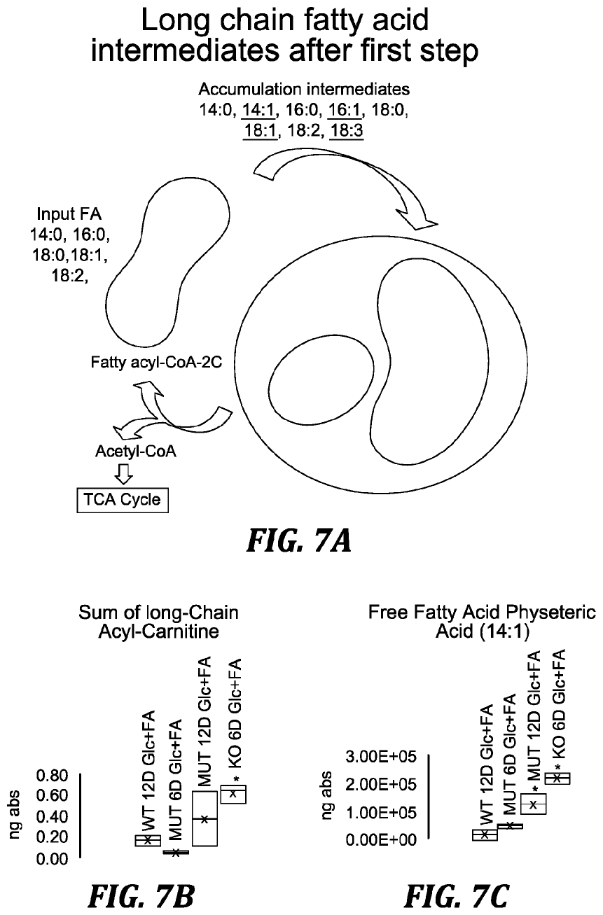

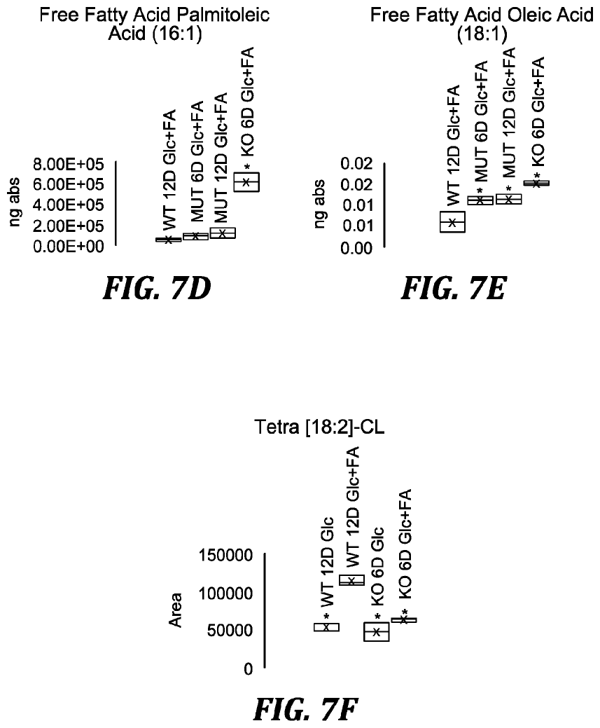

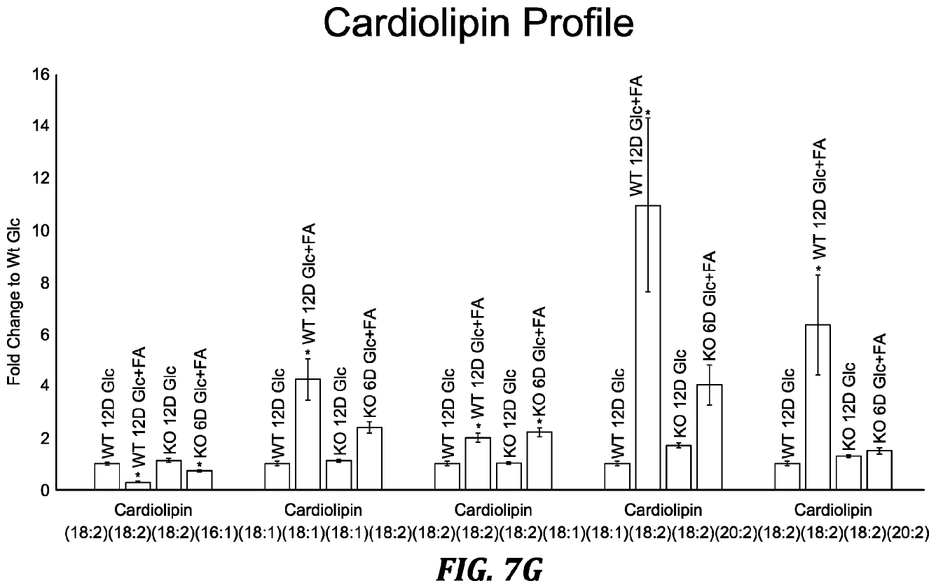

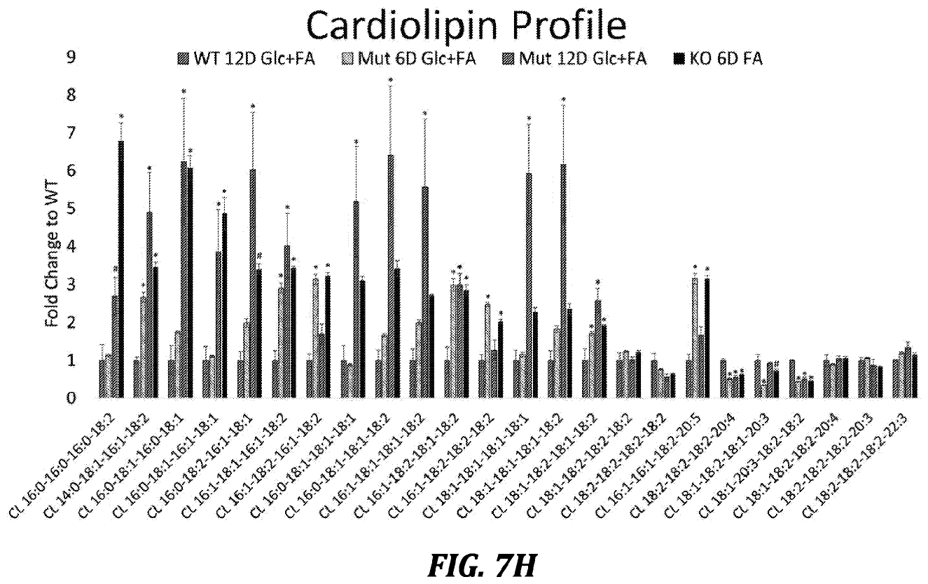

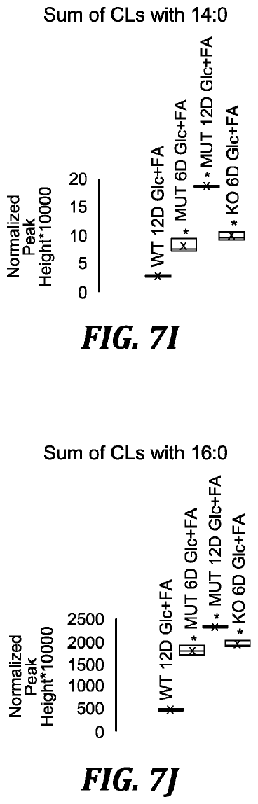

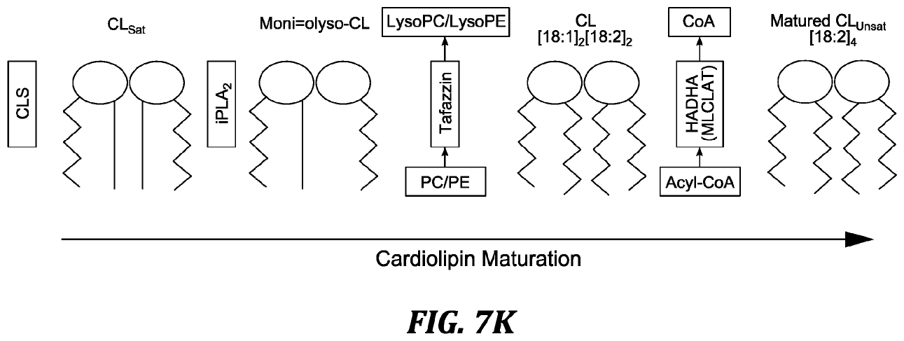

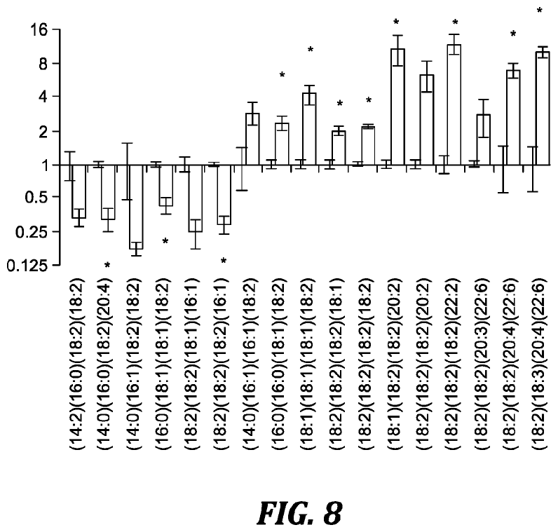

During the first step of fatty acid β-oxidation, acyl-CoA dehydrogenase generates a double bond between the alpha and beta carbons. Consequently, a disruption in HADHA should result in a build-up of FA intermediates after the first step (FIG. 7A). To assess the disruption of long-chain fatty acid oxidation in HADHA Mut and KO CMs, untargeted lipidomic analysis was performed to characterize global lipidomic changes.

There was an increase in long-chain acyl-carnitines in HADHA Mut and KO CMs as compared to WT CMs with no significant change in medium-chain acyl-carnitine levels (FIG. 7B; results for medium-chain acyl-carnitine levels not shown). These data suggest that a mutation in HADHA led to an accumulation of long-chain fatty acids in the mitochondria in the absence of HADHA. During the first step of long-chain FAO, saturated fatty acids are processed into fatty acids with a single double bond, for instance: 14:0→14:1, 16:0→16:1 and 18:0→18:1, while unsaturated fatty acids, on the carboxyl end, go through the first step of FAO and gain another double bond, for instance: 18:1→18:2 and 18:2→18:3. Accordingly, minimal variation was found in the levels of the saturated fatty acids: 14:0, 16:0 and 18:0 (not shown) but, large increases in the abundance of 14:1, 16:1, 18:1 in the HADHA Mut and KO CMs along with slight increases in 18:2 and 18:3 in the HADHA KO CMs (FIGS. 7C-7E; results for 18:2 and 18:3 in the HADHA KO CMs not shown). These data show that disruption and KO of HADHA leads to a specific long-chain FA intermediate accumulation. Yet, one of the striking phenotypes that were observed were rounded and collapsed mitochondria and not bursting mitochondria due to potential fatty acid overload. Therefore the next step was to examine another phospholipid category that regulates mitochondrial structure, cardiolipins.

HADHA and TAZ Act in Series to Bring about Mature Cardiolipin Remodeling

Cardiolipin (CL) is a phospholipid essential for optimal mitochondrial function and homeostasis as it maintains electron transport chain function along with other mitochondrial functions. CL is the major phospholipid of the mitochondrial inner membrane that is synthesized in the mitochondria and is dynamically remodeled during postnatal development and disease [see, e.g., Kiebish, M. A., et al., Myocardial regulation of lipidomic flux by cardiolipin synthase: setting the beat for bioenergetic efficiency. J Biol Chem, 2012. 287(30): p. 25086-97; and He, Q. and X. Han, Cardiolipin remodeling in diabetic heart. Chem Phys Lipids, 2014. 179: p. 75-81]. The most abundant species of CL in the human heart is tetralinoleoyl-CL (tetra[18:2]-CL). In cardiac diseases such as diabetes, ischemia/reperfusion and heart failure, or due to a specific mutation in a cardiolipin remodeling enzyme tafazzin (TAZ; which leads to Barth syndrome), tetra[18:2]-CL levels are abnormal. Specific cardiolipin maturation is observed in the early postnatal mouse heart. The inventors have used the maturation paradigm to reach the maturation step of iPSC derived cardiomyocytes (CM) that allows utilization of fatty acids as energy source. Various maturation paradigms were identified that were able to mimic not only FAO steps but also Cardiolipin (CL) early post-natal remodeling process (FIG. 8). Using targeted mass spec lipidomic analysis, maturation in wild type (WT) CMs were shown to result in a significant increase in tetra[18:2]-CL, similar to previously observed findings during in vivo cardiomyocyte postnatal maturation. These data show that CL maturation in cardiomyocytes can be induced in vitro. Furthermore, as shown in early postnatal in vivo development, WT CMs shift their CL profile by decreasing most CLs with [14:0],[14:1],[16:1] and [16:0] (FIG. 8) and increasing CLs with acylchains greater than 18 carbons, including the intermediate [18:1][18:2][18:2][20:2] (FIG. 8). While this CL maturation did not reach that of the adult CL remodeling stage, the post-natal maturation observed serves as a useful assay for interrogating, with the goal of ultimately understanding and manipulating the first steps in CL maturation in CM, in normal and pathological mutant12-21 situations. Using targeted lipidomics, WT CMs were analyzed supplemented with and without FAs. FA treated WT CMs resulted in a significant increase in tetra[18:2]-CL (FIG. 7F), similar to previously observed findings during in vivo cardiomyocyte postnatal maturation [see, Kiebish, M. A., et al., J Biol Chem, 2012. 287(30): p. 25086-97; and He, Q. and X. Han, Chem Phys Lipids, 2014. 179: p. 75-81, supra]. These data show that CL maturation in cardiomyocytes can be induced in vitro. However, the HADHA KO CMs, after FA treatment, were unable to increase the amount of tetra[18:2]-CL as compared to WT FA treated CMs. Furthermore, as shown in postnatal in vivo development, WT CMs shift their CL profile to a more mature CL profile showing a significant decrease in CLs with [16:1] and increased CLs with carbons greater than 18, including the intermediate [18:1][18:2][18:2][20:2] [see, Kiebish, M. A., et al., J Biol Chem, 2012. 287(30): p. 25086-97]. However, HADHA KO CMs were unable to remodel their CL profiles as efficiently as WT CMs (FIG. 7G). These data show that, surprisingly, HADHA, in addition to its role in long-chain FAO, is also required for the cardiomyocyte CL remodeling process.

Since HADHA KO CMs showed a CL remodeling defect, the cardiolipin species were next analyzed in more detail in WT, HADHA Mut and KO CMs using full lipidomics. Reinforcing our targeted lipidomics results, we found that HADHA Mut and KO CMs challenged with FAs showed an increased abundance of lighter chain CLs and a depletion of heavier chain CLs (FIG. 7H). Three CL species, tetra[18:1], [18:1][18:1][18:1][18:2] and [18:1][18:1][18:2][18:2] were significantly enriched in the HADHA Mut and KO CMs (FIG. 7H). Interestingly, [18:1][18:1][18:2][18:2] CL is specifically depleted in Barth syndrome patients who have a mutation in TAZ.

It has been previously shown that the HADHA protein has a similar enzymatic function to monolysocardiolipin acyltransferase (MLCL AT). MLCL AT transfers mainly unsaturated fatty acyl-chains to lyso-CL. It therefore seems plausible that HADHA has a direct role in remodeling cardiolipin to produce mature tetra[18:2]-CL species in cardiomyocytes. If TAZ and HADHA are acting in parallel to produce remodeled CL, they should both be equally depleting the MLCL pool. When TAZ is KO'd, there is a dramatic increase in MLCL, showing the direct usage of MLCL by TAZ to generate mature CL. However, it was observed here that when HADHA is KO'd, there is no change in the MLCL pool (not shown). This suggests that HADHA does not remodel MLCL but rather CL. If TAZ and HADHA are acting in parallel, the KO of each should not result in the inverse accumulation relationship to specific CL intermediates. For instance, TAZ KO results in the decrease of [18:1][18:1][18:2][18:2] CL. Yet in the current HADHA KO an accumulation of the same species is observed. Accordingly, it is proposed that TAZ first remodels MLCL to an intermediate of CL such as [18:1][18:1][18:2][18:2] and then HADHA continues to remodel the CL species to tetra[18:2]-CL.

Loss of HADHA Function does not Augment ALCAT1 Function

To garner a better understanding of how the cardiolipin profile was changing due to the lack of HADHA, which new CL species became enriched in the HADHA Mut and KO CMs was investigated. CL species that had fatty-acid acyl-chains of saturated fatty-acids, such as 14:0 and 16:0, were enriched in the HADHA Mut and KO CMs (FIG. 7I, J). No CL acyl-chains that had 18:0 were identified. Typically, nascent CL with multiple saturated fatty-acid acyl-chains (CL_{Sat}), have been synthesized from cardiolipin synthase (CLS) (see, e.g., FIG. 7K). During the remodeling process of CL_{Sat}, the saturated fatty-acid acyl-chains are replaced by unsaturated fatty-acid acyl-chains. These data suggest a nascent CL_{Sat} accumulation in HADHA mutants.

We next examined ALCAT1 as a means for the HADHA Mut and KO CMs to utilize for CL remodeling. Since ALCAT1 has no preference for fatty-acyl substrate, it should utilize whichever fatty-acyl-CoA substrate is present. Hallmarks of ALCAT1 activity are an increase in polyunsaturated fatty-acid acyl-chains being incorporated to CL. However, when the CL species that had acyl-chains with fatty-acids with a carbon length 20 or greater were examined, the majority of the HADHA Mut and KO CMs actually had less species as compared to WT CMs (FIG. 7H). Furthermore, there was no increase in CL species that had multiple acyl-chains with fatty-acids with a carbon length 20 or greater in any of the groups. Consequently, these data suggest that ALCAT1 is not being engaged in the HADHA Mut and KO CMs to compensate for the loss of HADHA.

### Discussion

This investigation involved development of the first human MTP deficient cardiac model in vitro utilizing MiMaC matured hiPSC-CMs and resulted in the discovery that a TFPa/HADHA defect in long-chain FAO and CL remodeling results in disease like erratic beating suggesting a pro-arrhythmic state. Furthermore, a mechanism of action was demonstrated; mutations in HADHA resulted in abnormal composition of the prominent phospholipid, cardiolipin due to its acyl-CoA transferase activity. Abnormal CL composition results in defective mitochondrial cristae and highly reduced mitochondrial proton gradient. These mitochondrial defects manifested as sarcomere dissolution, defective calcium handling and electrophysiology. Delayed calcium storage and repolarization contributed to the uneven cardiomyocyte beating patterns, which in turn can precipitate tissue level arrhythmia seen in MTP deficient newborns with SIDS.

The study of MTP deficiency using pluripotent stem cell derived cardiomyocytes necessitated the generation of a tool that rapidly and efficiently matures cardiomyocytes in vitro to a stage that manifests post-natal cardiac FAO diseases. Many tools have been generated to mature hPSC-CMs which include: electrical and/or mechanical stimulation, cell microenvironment and culture time. However, none of these methods directly affect maturation aspects that allow the analysis of FA metabolism. Building off the inventors' preliminary work studying the role of Let-7 in hPSC-CM maturation, a microRNA maturation cocktail (MiMaC) was developed that was able to mature hPSC-CM size, force of contraction and metabolism. MiMaC facilitated the study of MTP deficiency in hPSC-CMs and is a potent tool that can be used to mature hPSC-CMs for the study of FAO disorders. Furthermore, the MiMaC system was used to better understand the late development, maturation processes. Importantly, a common microRNA target, HOPX, was discovered as a novel, critical regulator of cardiomyocyte maturation.

Previous studies have shown an increase in metabolic gene expression when cardiomyocytes develop from fetal to adult stage, metabolic remodeling. Increase of OXPHOS gene expression may indicate an increase in mitochondrial copy number or biosynthesis of a more mature mitochondria, or both. These scRNA-seq studies discovered a novel, intermediate cardiomyocyte sub-group with high metabolic gene expression for OXHPOS and Myc targets. These data suggest a possible intermediate stage from a fetal like CM to a more mature CM, which requires transient up-regulation of OXPHOS genes. Since Parkin is upregulated at this stage as well, these data support the hypothesis that quality control type mitophagy of fetal stage mitochondria and biosynthesis of mature mitochondria takes place in MiMaC induced cardiomyocyte maturation. This is similar to the previously shown mitophagy mediated response via Parkin during perinatal mouse heart development. Importantly, this intermediate stage in maturation was also observed in MTP/HADHA mutant cardiomyocytes, prior to development of the pathological state. Further dissection of this stage will allow mechanistic understanding of the regulation of this process both in normal and disease states.

Using pluripotent stem cell derived cardiomyocytes we searched for the etiology of the arrhythmia observed in patients with HADHA mutations, causing MTP deficiency. Importantly, hiPSC derived HADHA mutant cardiomyocytes recapitulated the arrhythmic phenotype observed in patients, emphasizing the utility of hiPSC-CMs for modeling human disease. To better understand the cause of the arrhythmia, the phenotype was assessed using fatty-acid challenged HADHA cardiomyocytes and identified a potential clue for the disease progression, cardiolipin. One novel therapeutic intervention that rescued part of the HADHA mutant phenotype was SS-31. SS-31 is a mitochondrial targeted peptide that has been shown to bind cardiolipin and prevent cardiolipin conformation changes under stress such as peroxidation [Birk, A. V., et al., The mitochondrial-targeted compound SS-31 re-energizes ischemic mitochondria by interacting with cardiolipin. J Am Soc Nephrol, 2013. 24(8): p. 1250-61]. SS-31 has been shown to inhibit mitochondrial depolarization and swelling in cardiac cells and islets and rescue cardiolipin defects in cardiomyocytes. Since it was found in this study that SS-31 rescued one aspect of the mitochondrial pathology, i.e., increased proton leak, and since abnormal CL species were observed in HADHA Mut CMs, it is proposed that cardiolipin defects precipitate the observed mitochondrial dysfunctions.

Cardiolipins are a critical component of the mitochondrial inner membrane. CL is an atypical phospholipid composed of four (instead of two) acyl-chains that are connected with a glycerol moiety. This atypical structure of cardiolipin results in a conical shape that is thought to be critical for inner mitochondrial membrane structure and function. In particular, cardiolipin has been shown to function in organizing the electron transport chain (ETC) higher order structure, important for ETC activity, and acts as a proton trap on the outer leaflet of the inner mitochondrial membrane. Hence, the reduction of the mature form of CL results in mitochondrial abnormalities such as proton gradient loss, ETC depression resulting in depressed ATP production and abnormal mitochondrial architecture.

Pathological remodeling of CL has been implicated in the mitochondrial dysfunction observed in diabetes, heart failure, neurodegeneration, and aging. However, the pattern and composition of abnormal CL species in the case of the HADHA Mut and KO CMs were more specific than seen previously in heart failure or diabetes, suggesting that HADHA may be directly involved in CL processing. Interestingly, previous studies using HeLa cells have suggested HADHA exhibits acyl-CoA transferase activity upon MLCL for its remodeling into cardiolipin. As such, these data suggest that defects in HADHA directly cause impaired cardiolipin remodeling resulting in the inability to produce and possibly maintain the acyl-chain composition of mature cardiolipin. However, the exact contribution of this acyltransferase to physiological CL remodeling has been unclear. It is now reported here that, in the described FA challenged human HADHA Mut and KO cardiomyocytes, mature tetra[18:2]-CL was reduced and mitochondrial activity was compromised. This is similar to previously seen findings in TAZ mutant causing Barth's syndrome, an X-linked cardiac and skeletal mitochondrial myopathy. These data, for the first time, establish the exact contribution of HADHA acyltransferase to physiological CL remodeling in human cardiomyocytes.

TAZ is a transacylase that is essential for the remodeling of MLCL to mature cardiolipin. Both HADHA and TAZ play key roles in generating mature cardiolipin and both diseases have similar pathological phenotypes including sudden unexplained death due to ventricular arrhythmias. Cardiolipin species [18:1][18:1][18:2][18:2], which is specifically reduced in abundance in TAZ mutants, showed an increased abundance in the described HAHDA Mut and KO CMs. Furthermore, there was no observed accumulation of MLCL in the HADHA Mut and KO CMs, which typically occurs when there are mutations in TAZ. These data suggest that CL remodeling is the result of first processing by TAZ and then by HADHA to generate tetra[18:2]-CL in human cardiomyocytes (FIG. 7K).

Mutations in HADHA clearly led to the inability of the CM to generate large amounts of tetra[18:2]-CL. It is also clear from the literature that once tetra[18:2]-CL species begin to deplete, CMs can fall into a pathological state of mitochondrial disarray. What is interesting about the present findings is that, as long as HADHA Mut and KO CMs are not challenged with FAs, they do not enter a disease state, even though they have less tetra[18:2]-CL. It is also demonstrated here that the addition of FAs to HADHA Mut and KO CMs does result in a long-chain FA accumulation. However, this FA accumulation does not lead to mitochondrial swelling and eventual rupture. Instead, it was found that the mitochondria collapse had become rounded in HADHA mutant. These data suggest that FAO phenotypes alone might not explain the defects observed in HADHA Mut and KO CMs and furthermore, CL remodeling is particularly important during the CM maturation process.

When the full panel of CL species was examined in CM, it became clear that the HADHA Mut and KO CMs could not achieve a mature CL profile by properly remodeling the CL side chains to [18:2] during CM maturation. Furthermore, long carbon groups of 20 or greater were not being aggressively incorporated multiple times in CL, suggesting ALCAT1 was not compensating. What was apparent was the presence of saturated FA side chains, 14:0 and 16:0 in CL. It is possible that the accumulation of CL species in the HADHA Mut and KO with saturated side chains resulted in the collapse of the mitochondrial structure and cardiac pathology that follows. Hence, these data suggest that a mutation in the HADHA enzyme during CM maturation process results in an over accumulation of immature CL-saturated species, that may be causal for the mitochondrial defects and pathology seen in HADHA CMs (FIG. 7K).

Here it is demonstrated that long-chain fatty acids, the normal substrates used to generate energy and phospholipids in postnatal and adult CMs, precipitate the MTP deficient pathology in CMs leading to an abnormal cardiolipin pattern that resulted in severe mitochondrial defects and calcium abnormalities that pre-dispose CMs to erratic beating in HADHA Mut CMs. SS-31 was identified as a novel therapy to rescue the proton leak phenotype of FA challenged HADHA Mut CMs. This demonstrates that SS-31, or other cardiolipin-affecting compounds, can serve as a potential treatment to mitigate aspects of mitochondrial dysfunction in MTP deficiency.

### Examples

The following methods are set forth so as to provide those of ordinary skill in the art with a complete disclosure and description of how to make and use embodiments of the disclosed innovations, and are not intended to limit the scope of what the inventors regard as their invention nor are they intended to represent that the experiments below are all or the only experiments performed.

Methods

hESC and hiPSC and Cardiac Differentiation

The hESC line RUES2 (NIHhESC-09-0013) and hiPSC line WTC #11, previously derived in the Conklin laboratory [Kreitzer, F. R., et al., A robust method to derive functional neural crest cells from human pluripotent stem cells. Am J Stem Cells, 2013. 2(2): p. 119-31], were cultured on Matrigel growth factor-reduced basement membrane matrix (Corning) in mTeSR media (StemCell Technologies). A monolayer-based directed differentiation protocol was followed to generate hESC-CMs and hiPSC-CMs, as done previously [Palpant, N.J., et al., Generating high-purity cardiac and endothelial derivatives from patterned mesoderm using human pluripotent stem cells. Nat Protoc, 2017. 12(1): p. 15-31]. hiPSC-CM cardiolipin assay was done with a small molecule monolayer-based directed differentiation protocol, as done previously [Burridge, P. W., et al., Chemically defined generation of human cardiomyocytes. Nat Methods, 2014. 11(8): p. 855-60]. 15 days after differentiation hPSC-CMs were enriched for the cardiomyocyte population using a lactate selection process [Tohyama, S., et al., Distinct metabolic flow enables large-scale purification of mouse and human pluripotent stem cell-derived cardiomyocytes. Cell Stem Cell, 2013. 12(1): p. 127-37]. Cardiomyocyte populations were generated ranging from 40-60% that were then enriched to 75-80% cardiomyocytes after 4 days of lactate enrichment.

HADHA Line Creation

Using LentiCrisprV2 plasmid [Sanjana, N. E., O. Shalem, and F. Zhang, Improved vectors and genome-wide libraries for CRISPR screening. Nat Methods, 2014. 11(8): p. 783-4] (Addgene plasmid #52961) two different gRNAs targeted to Exon 1 of HADHA were designed using CRISPRScan [Moreno-Mateos, M. A., et al., CRISPRScan: designing highly efficient sgRNAs for CRISPR-Cas9 targeting in vivo. Nat Methods, 2015. 12(10): p. 982-8]. Sequences for the gRNAs can be found in Table 1. The gRNA and Cas9 expressing plasmids were transiently transfected into the WTC line using GeneJuice (EMD Millipore). 24 hours after transfection, WTCs were puromycin selected for two days and then clonally expanded. DNA of the clones was isolated, the region around the targeting guides was PCR amplified (see guides in Table 1) and sequenced to determine the insertion and deletion errors generated by CRISPR-Cas9 system in exon 1 of HADHA. Western analysis was performed to determine the levels of HADHA protein in HADHA mutants. 31 clones were sent for sequencing from gRNA1 experiment, 6 clones (19%) had no mutations while 25 clones (81%) were found to have mutations. 24 clones were sent for sequencing from gRNA2, 1 clone had no mutations (4%) while 23 clones (96%) were found to have mutations. Two of the mutant lines were analyzed further in this study.

TABLE 1
gRNA for LentiCRISPRV2 (SEQ ID NOS are in parentheses)

| Name | Forward Primer | Reverse Primer |
|---|---|---|
| 205-5p | CACCGCCGGAGTCTGTCTCATACCC | AAACGGGTATGAGACAGACTCCGG |
| | (15) | C (16) |
| 200a-3p | CACCGAGCTTGACTCTAACACTGTC | AAACGACAGTGTTAGAGTCAAGCTC |
| | (17) | (18) |
| 122-5p | CACCGAGTTTCCTTAGCAGAGCTG | AAACCAGCTCTGCTAAGGAAACTC |
| | (19) | (20) |
| HADHA | CACCGGGCAGGCCTCACCTCGGGA | AAACCTCCCGAGGTGAGGCCTGCCC |
| Ex1 | G (21) | (22) |
| gRNA1 | | |
| HADHA | CACCGGGAAGGCAGAAAAGCGGCT | AAACCAGCCGCTTTTCTGCCTTCCC |
| Ex1 | G (23) | (24) |
| gRNA 2 | | |

CRISPR Off-Target

The potential off targets of the HADHA gRNA were identified using Crispr-RGEN's Cas-OFFinder tool [Bae, S., J. Park, and J. S. Kim, Cas-OFFinder: a fast and versatile algorithm that searches for potential off-target sites of Cas9 RNA-guided endonucleases. Bioinformatics, 2014. 30(10): p. 1473-5]. The top predicted off targets were then amplified by GoTaq PCR and sequenced. Off-target primers can be found in Table 2.

TABLE 2
Primers for off-target analysis (SEQ ID NOS are in parentheses)

| Name | Forward Primer | Reverse Primer |
|---|---|---|
| HADHA_OFFT_1 | CGTACGTGTTCTGCACAGCC | CAGCAATGTTCTGAAGGCCC |
| | (25) | (26) |
| HADHA_OFFT_2 | CCTGCCCCCTTCAAGGTAAG | CTGGTCTGATAGGTGGGGGA |
| | (27) | (28) |
| HADHA_OFFT_3 | GAGAGCTAGGCTTTGTGCCA | CTTATGTGGCCCCGTGTTCT |
| | (29) | (30) |

RNA Extraction and qPCR Analysis

RNA was extracted from cells using Trizol and analysed with SYBR green qPCR using the 7300 real-time PCR system (Applied Biosystems). Primers used are listed in Table 3. Linear expression values for all qPCR experiments were calculated using the delta-delta Ct method.

TABLE 3
Quantitative RT-qPCR primers for human genes (SEQ ID NOS are in
parentheses)

| Gene | Forward Primer | Reverse Primer |
|---|---|---|
| Name | | |
| MYL2 | TGTCCCTACCTTGTCTGTTAGCCA | ATTGGAACATGGCCTCTGGATGGA |
| | (31) | (32) |
| TNNC1 | TGGTTCGGTGCATGAAGGAC (33) | GTCGATGTAGCCATCAGCATT (34) |
| GAPDH | CTGGGCTACACTGAGCACC (35) | AAGTGGTCGTTGAGGGCAATG (36) |
| NPPA | CAACGCAGACCTGATGGATTT (37) | AGCCCCCGCTTCTTCATTC (38) |

Protein Extraction and Western Blot Analysis

Cells were lysed directly on the plate with a lysis buffer containing 20 mM Tris-HCl pH 7.5, 150 mM NaCl, 15% Glycerol, 1% Triton X-100, 1M β-Glycerolphosphate, 0.5M NaF, 0.1M Sodium Pyrophosphate, Orthovanadate, PMSF and 2% SDS [Moody, J. D., et al., First critical repressive H3K27me3 marks in embryonic stem cells identified using designed protein inhibitor. Proc Natl Acad Sci USA, 2017]. 25 U of Benzonase Nuclease (EMD Chemicals, Gibbstown, N.J.) was added to the lysis buffer right before use. Proteins were quantified by Bradford assay (Bio-Rad), using BSA (Bovine Serum Albumin) as Standard using the EnWallac Vision. The protein samples were combined with the 4× Laemmli sample buffer, heated (95° C., 5 min), and run on SDS-PAGE (protean TGX pre-casted 4%-20% gradient gel, Bio-Rad) and transferred to the Nitro-Cellulose membrane (Bio-Rad) by semi-dry transfer (Bio-Rad). Membranes were blocked for lhr with 5% milk and incubated in the primary antibodies overnight at 4° C. The membranes were then incubated with secondary antibodies (1:10000, goat anti-rabbit or goat anti-mouse IgG HRP conjugate (Bio-Rad) for lhr and the detection was performed using the immobilon-luminol reagent assay (EMD Millipore). Primary antibodies are as follows: Alpha tubulin antibody Cell Signalling Technologies (2144) 1:2000, Beta tubulin Promega (G7121) anti-mouse 1:4000, Beta Actin Cell Signalling Technologies (4970) 1:4000, HADHA Abcam (ab54477 anti-rabbit 1:1000, UCP3 Abcam (ab3477) anti-rabbit 1:200, SLC25A4 (ANTI) Sigma (SAB2105530) anti-rabbit 1:1000, OXPHOS MitoSciences (MS604/G2830) anti-mouse 1:1000, anti-GFP Invitrogen (A-11122) anti-rabbit 1:1000.

microRNA Overexpression and Knockout

LentiCrisprV2 plasmid (Addgene 52961) was used to knockout (KO) microRNAs-141, -200a, -205 and -122. gRNAs for each miR that had either the protospacer adjacent motif (PAM) NGG cut site adjacent or in the seed region of the mature microRNA were chosen to test. gRNAs can be found in Table 1. The global reduction of each miR was assessed via TaqMan RT-qPCR with probes specific against the mature form of each respective miR.

The pLKO.1 TRC vector (pLKO.1—TRC cloning vector (Addgene plasmid #10878) was used to overexpress (OE) a microRNA [Moffat, J., et al., A lentiviral RNAi library for human and mouse genes applied to an arrayed viral high-content screen. Cell, 2006. 124(6): p. 1283-98]. The genomic sequence 200 bp up- and down-stream of the mature microRNA was amplified and purified. Primers for each microRNA can be found in Table 4. The amplicons were cloned between Agel and EcoRI sites of pLKO.1 TRC vector under the human U6 promoter.

TABLE 4
Primers for genomic amplification of microRNA region (SEQ ID NOS
are in parentheses)

| Name | Forward Primer | Reverse Primer |
|---|---|---|
| miR-208b | CCGGTGAGTTCTGAGCAGCCTGACT | ATCCTCTGCCTGATGTTCTCGAATT |
| | T (39) | C (40) |
| miR-452 | AAAAAACCGGTCTCACACGAGCTC | AAAAAGAATTCCAACCCCAGTTGG |
| | CATTCCC (41) | TAAGCGT (42) |
| miR-378e | ACTAGGACGAGCTAGTGGGG | ACCCAAAGTGTACAATCATTGACT |
| | (43) | (44) |

Viral Production

HEK 293FT cells were plated one day before transfection. On the day of transfection, the OE or KO plasmid of choice was combined with packaging vectors psPAX2 (psPAX2 was a gift from Didier Trono Addgene plasmid #12260) and pMD2.G (pMD2.G was a gift from Didier Trono Addgene plasmid #12259) in the presence of 1 μg/μL of polyethylenimine (PEI) per 1 μg of DNA. Medium was changed 24 hours later and the lentiviruses were harvested 48 and 72 hours after transfection. Viral particles were concentrated using PEG-it (System Biosciences, Inc).

hiPSC-CM Transduction and Selection

hiPSC-CMs were transduced on day 14 post-induction in the presence of hexadimethrine bromide (Polybrene, 6 μg/ml). Lentivirus was applied for 17-24 hours and then removed. Cells were cultured for an additional two weeks. Lactate selection was employed to obtain an enriched population of cardiomyocytes [Tohyama, S., et al., Distinct metabolic flow enables large-scale purification of mouse and human pluripotent stem cell-derived cardiomyocytes. Cell Stem Cell, 2013. 12(1): p. 127-37]. Puromycin selection was used to select for cells that have positively incorporated the vector. After two weeks of culture, cells were harvested for end point analysis. For the MiMaC group, hiPSC-CMs were transduced with a lower dose of the four different lentiviruses concurrently while controls were transduced with both control vectors: pLKO.1 and the LentiCRISPRv2 empty vector.

Immunocytochemistry and Morphological Analysis

Cells were fixed in 4% (vol/vol) paraformaldehyde, blocked for an hour with 5% (vol/vol) normal goat serum (NGS), and incubated overnight with primary antibody in 1% NGS, followed by secondary antibody staining in NGS. Measurements of CM area were performed using Image J software. Quantification of mitotracker intensity were performed using Image J software and following previously published methods on colocalization quantification [Li, Q., et al., A syntaxin 1, Galpha(o), and N-type calcium channel complex at a presynaptic nerve terminal: analysis by quantitative immunocolocalization. J Neurosci, 2004. 24(16): p. 4070-81]. Analysis was done on a Leica TCS-SPE Confocal microscope using a 40× or 63× objective and Leica Software. Primary antibodies used were: αActinin 1:250 Sigma A7811 anti-mouse, HADHA 1:250 abcam ab54477 anti-rabbit, ATP Synthase β 1:250 abcam ab14730 anti-mouse, Titin 1:300 Myomedix TTN-9 (cTerm) anti-rabbit, GFP 1:300 Invitrogen A-11122 anti-rabbit. Secondary antibodies and other reagents used were: DAPI at a concentration of 0.02 m/mL, phalloidin alexa fluor 568 1:250, alexa fluor 488 or 647-conjugated goat anti-mouse and anti-rabbit secondary antibodies 1:500 (Molecular Probes). MitotrackerCMTMRos Life technologies (M7510) used at a final concentration of 300 nM in RPMI with B27 plus insulin supplement, incubated with cells for 45 minutes prior to fixation.

Micro-Electrode Array

Electrophysiological recording of spontaneously beating cardiomyocytes was collected for 2 minutes using the AxIS software (Axion Biosystems). After raw data collection, the signal was filtered using a Butterworth band-pass filter and a 90 μV spike detection threshold. Field potential duration was automatically determined using a polynomial fit T-wave detected algorithm.

Microposts (Force of Contraction and Beat Rate)

Arrays of polydimethylsiloxane (PDMS) microposts were fabricated as previously described [Beussman, K. M., et al., Micropost arrays for measuring stem cell-derived cardiomyocyte contractility. Methods, 2016. 94: p. 43-50]. The tips of the microposts were coated with mouse laminin (Life Technologies), and cells were seeded onto the microposts in Attofluor® viewing chambers (Life Technologies) at a density of approximately 75,000 per cm² in RPMI medium with B27 supplement and 10% fetal bovine serum. The following day, the media was removed and replaced with serum-free RPMI medium, which was exchanged every other day. Once the cells resumed beating (typically 3 to 5 days after seeding), contractions of individual cells were imaged (at a minimum of 70 FPS) using a Hamamatsu ORCA-Flash2.8 Scientific CMOS camera fitted on a Nikon Eclipse Ti upright microscope using a 60× water immersion objective. Prior to imaging, the cell culture media was replaced with a Tyrode buffer containing 1.8 mM Ca2+, and a live cell chamber was used to maintain the cells at 37° C. throughout the imaging process. A custom-written matlab code was used to track the deflection, Δᵢ, of each post i underneath an individual cell, and to calculate the total twitch force, F_{twitch}=Σᵢ₌₁^{# posts} kₚₒₛₜ×Δᵢ [Beussman, K. M., et al., Methods, 2016. 94: p. 43-50], where kₚₒₛₜ=56.5 nN/μm and the spacing between posts was 6 μm.

Seahorse Assay

The Seahorse XF96 extracellular flux analyzer was used to assess mitochondrial function as previously described [Kuppusamy, K. T., et al., Let-7 family of microRNA is required for maturation and adult-like metabolism in stem cell-derived cardiomyocytes. Proc Natl Acad Sci U.S.A., 2015]. The plates were pre-treated with 1:60 diluted Matrigel reduced growth factor (Corning). At around 28 days after differentiation, cardiomyocytes were seeded onto the plates with a density of 50,000 cells per XF96 well. The seahorse assays were carried out 3 days after the seeding onto the XF96 well plate. One hour before the assay, culture media was exchanged for base media (unbuffered DMEM; Seahorse XF Assay Media) supplemented with sodium pyruvate (Gibco/Invitrogen, 1 mM) and with 25 mM glucose (for MitoStress assay), 25 mM glucose with 0.5 mM Carnitine for Palmitate assay. Injection of substrates and inhibitors were applied during the measurements to achieve final concentrations of 4-(trifluoromethoxy) phenylhydrazone at 1 μM (FCCP; Seahorse Biosciences), oligomycin (2.5 μM), antimycin (2.5 μM) and rotenone (2.5 μM) for MitoStress assay; 200 mM palmitate or 33 μM BSA, and 50 μM Etomoxir (ETO) for palmitate assay. The OCR values were further normalized to the number of cells present in each well, quantified by the Hoechst staining (Hoechst 33342; Sigma-Aldrich) as measured using fluorescence at 355 nm excitation and 460 nm emission. Maximal OCR is defined as the change in OCR in response to FCCP compared to OCR after the addition of oligomycin. ATP production was calculated as the difference between the basal respiration and respiration after oligomycin. Proton leak was calculated as the difference between respiration after oligomycin and after antimycin & rotenone. Cellular capacity to utilize palmitate as an energy source was calculated as the difference between the average OCR after second palmitate addition and the final respiration value before the second addition of palmitate. The reagents were from Sigma, unless otherwise indicated.

RNA-Sequencing

Day-30 hiPSC-CMs were harvested for RNA preparation and genome wide RNA-seq (>20 million reads). RNA-seq samples were aligned to hg19 using Tophat, version 2.0.13 [Trapnell, C., L. Pachter, and S. L. Salzberg, TopHat: discovering splice junctions with RNA-Seq. Bioinformatics, 2009. 25(9): p. 1105-11]. Gene-level read counts were quantified using htseq-count [Anders, S., P. T. Pyl, and W. Huber, HTSeq—a Python framework to work with high-throughput sequencing data. Bioinformatics, 2015. 31(2): p. 166-9] using Ensembl GRCh37 gene annotations. Genes with total expression above 1 normalized read count across RNA-seq samples in each binary comparison were kept for differential analysis using DESeq [Anders, S. and W. Huber, Differential expression analysis for sequence count data. Genome Biol, 2010. 11(10): p. R106]. Princomp function from R was used for Principal Component Analysis. TopGO R package [Alexa, A., J. Rahnenfuhrer, and T. Lengauer, Improved scoring of functional groups from gene expression data by decorrelating GO graph structure. Bioinformatics, 2006. 22(13): p. 1600-7] was used for Gene Ontology enrichment analysis. To assess the effects of miR perturbation on cardiac maturation pathways, each condition was compared against their empty vector (EV), and up-regulated genes (>1.5 fold change) and down-regulated genes were identified (<−1.5 fold change). A hypergeometric test was performed on up- and down-regulated genes separately for enrichment against a curated set of pathways that are beneficial for cardiac maturation, resulting in a m by n matrix, where m is the number of pathways (m=7) and n is the number of conditions (n=6, including EV). The negative log 10 of the ratio between enrichment p-value for up- and down-regulated genes were calculated to represent the overall net “benefit” of a treatment: large positive value (>0) means the treatment results in more up-regulation of genes in cardiac maturation pathways than down-regulation of these genes, and more negative values means the treatment results in more down-regulation of genes in cardiac maturation pathways.

Single Cell RNA-Sequencing

Raw single cell RNA-seq data is processed through the CellRanger pipeline from 10× Genomics. Output of the CellRanger pipeline is further analyzed using Seurat R package [Satij a, R., et al., Spatial reconstruction of single-cell gene expression data. Nat Biotechnol, 2015. 33(5): p. 495-502]. Cells with more than 40% of reads mapped to mitochondrial genes, less than 200 detected genes or less than 2000 Unique Molecular Identifiers (UMIs) are removed. Remaining cells are scaled by number of UMIs and % mapped to mitochondrial genes. Parameters for tSNE analysis of maturation single cell RNA-seq data were 2905 top variable genes, top 10 principal components, and resolution 0.5. Parameters for tSNE analysis of HADHA mutant single cell RNA-seq data were 3375 top variable genes, top 10 principal components, and resolution 0.4. Cell cycle genes from Kowalczyk et al [Kowalczyk, M. S., et al., Single-cell RNA-seq reveals changes in cell cycle and differentiation programs upon aging of hematopoietic stem cells. Genome Res, 2015. 25(12): p. 1860-72] and the CellCycleScoring function in the Seurat package were used to assess the effects of cell cycle on clustering. Genes detected in at least 25% of cells in either cluster and have false discovery rate<0.1 are defined as differentially expressed. Expression values are normalized for each gene across all cells plotted in the heat maps (i.e., Z-scores). Human in vivo maturation markers are based on genes up-regulated in adult heart compared to fetal heart in the Roadmap Epigenomics Project [Roadmap Epigenomics, C., et al., Integrative analysis of 111 reference human epigenomes. Nature, 2015. 518(7539): p. 317-30]. Mouse in vivo maturation markers are based on genes up-regulated in the in vivo cardiomyocyte single cell RNA-seq data from DeLaughter et al [DeLaughter, D. M., et al., Single-Cell Resolution of Temporal Gene Expression during Heart Development. Dev Cell, 2016. 39(4): p. 480-490]. Genes significantly higher in adult heart compared to fetal were selected using DESeq (2 fold higher in adult, FDR <0.05). We then intersected these genes with the top 30 most highly expressed genes in each scRNA-seq cluster to get the final gene list for heatmap in FIG. 3O. Gene Ontology enrichment is performed using the TopGO package [Alexa, A., J. Rahnenfuhrer, and T. Lengauer, Improved scoring of functional groups from gene expression data by decorrelating GO graph structure. Bioinformatics, 2006. 22(13): p. 1600-7].

Calcium Transient Analysis Method

Cardiomyocytes were plated on Matrigel coated round glass coverslips. The cardiomyocytes were incubated for 25 minutes at 37° C. with 1 mM Fluo-4 AM (Life Technologies, F14201) in Tyrode's buffer (1.8 mM CaCl₂, 1 mM MgCl₂, 5.4 mM KCl, 140 mM NaCl, 0.33 mM NaH₂PO₄, 10 mM HEPES, 5 mM glucose, pH to 7.4). The substrate was then transferred to a 60 mm Petri dish fresh with pre-warmed Tyrode's buffer for imaging. Samples were imaged using a Hamamatsu ORCA-Flash2.8 Scientific CMOS camera fitted on a Nikon Eclipse Ti upright microscope. Videos were taken with a 40× water-immersion objective at a framerate of at least 20 frames per second. The fluorescence power was adjusted to ensure adequate capture of fluorescence change during depolarization without bleaching, and the same fluorescence power was used for all experiments. The cardiomyocytes were biphasically stimulated at 5 V/cm with carbon electrodes (Ladd Research, 30250) at either 0.5 Hz or 1 Hz, and at least 5 beats were captured during each video for analysis.

Videos were analyzed with a custom MATLAB code; calcium transients were obtained finding the cell boundary and averaging the fluorescence within the boundary for each video frame. The background fluorescence was determined automatically for each video frame and subtracted from the calcium transients. The calcium transients were then analyzed to find the peak fluorescence (F), baseline fluorescence (F₀), time to peak (Tₚₑₐₖ), and time to 50% and 90% relaxation (T_{50R}, T_{90R}). The rates to peak, 50%, and 90% relaxation (Rₚₑₐₖ, R_{50R}, R_{90R}) were calculated by dividing the respective fluorescence change by the respective time. An exponential decay function (e^{-t/τ}) was fit to the relaxation between 10% and 90% relaxation to determine the relaxation coefficient, T. All of these measurements were obtained for at least 4 beats in each video and averaged for comparison.

TEM

Cells were fixed in 4% glutaraldehyde in sodium cacodylate buffer, post fixed in osmium tetroxide, en bloc stained in 1% uranyl acetate, dehydrated through a series of ethanol, and embedded in Epon Araldite. 70 nm sections were cut on a Leica EM UC7 ultra microtome, and viewed on a JEOL 1230 TEM.

Glucose and Fatty Acid Media

The base media, which we are calling Glucose Media, is RPMI supplemented with B27 with insulin. The fatty acid media is the glucose media with oleic acid conjugated to BSA (Sigma 03008): 12.7 m/mL, linoleic acid conjugated to BSA (Sigma L9530): 7.05 m/mL, sodium palmitate (Sigma P9767) conjugated to BSA (Sigma A8806): 52.5 μM and L-carnitine: 125 μM.

Elamipretide (SS-31)

SS-31 came from Stealth BioTherapeutics and was dissolved in PBS. A final concentration of 1 nM was used in experiments.

Box Plots

The ‘x’ in each box plot denotes the average value while the horizontal bar denotes the median value, no outlier values are shown. * denotes P<0.05.

Bar Graphs

Bar graphs show the mean±SEM. Bar graphs which do not show SEM are generated from RNA-Sequencing data that had one or two samples sequenced.

STRING Analysis

Protein association maps were generated using STRING version 10.5. In each diagram, genes connected to one another have an association with one another. There are three action effects: arrow->positive, —|− negative and line with a circle on the end —unspecified. There are also eight different action types that are denoted by line color: green—activation, blue—binding, cyan—phenotype, black—reaction, red—inhibition, purple—catalysis, pink—post-translational modification and yellow—transcriptional regulation. Kmeans clustering was used to identify the significantly changed genes due to MiMaC for: muscle structure development and extracellular matrix organization. Markov Clustering Algorithm (MCL) was used to identify genes MiMaC had down-regulated to control cell division.

Statistical Analysis

Statistical analysis was performed on experiments with an N equal or greater to 3. P values were calculated using student t-test or one-way ANOVA. For student t-test a Shapiro-Wilk normality test was performed. For one-way ANOVA a Kolmogorov-Smirnov normality test was performed. For multiple comparisons, the Holm-Sidak method was used. For one-way ANOVA analysis that failed the normality test, ANOVA a Kruskal-Wallis one-way ANOVA of Variance on Ranks was performed. For multiple comparisons, the Dunn's method was used. All statistical tests used an α=0.05.

Targeted Cardiolipin Analysis Using LC-MS/MS

Wildtype (WT) hiPSC-CMs treated for 12D Glc+FA media and HADHA Mut hiPSC-CMs treated for 6D and 12D Glc+FA media were used. Immediately before extraction, each cell pellet was dissolved in 40 μL DMSO and the membranes were disrupted by sonication. Cells were subjected to sonication using 3 cycles consisting of 20 seconds on, 10 seconds off. Care was taken to keep the cells on ice during sonication. After shaking, the suspension was transferred into a 2 mL glass LC vial.

For cardiolipin extraction, an extraction mixture consisting of 20 mL chloroform/methanol mix (2:1 v/v) and 304, internal standard solution (5 mg PC (18:0/18:1(9Z)) (Avanti Polar Lipids, Inc., Alabaster, Ala.) was prepared. Next, 600 μL of the extraction mixture was added to the samples, followed by vortexing and incubation at −20° C. for 20 minutes. The samples were then sonicated in an ice bath for 15 minutes. Purified water (100 μL) was added, and the samples were shaken for 30 minutes at room temperature. After centrifugation at 12,000×g for 10 minutes at 4° C., the bottom phase was transferred to a new glass LC vial and dried under vacuum. The residue was then reconstituted by adding 150 μL acetonitrile/isopropanol/H₂O (65:30:5, v/v/v), and centrifuged at 20,000×g for 10 minutes at 4° C. The supernatant was transferred to individual glass vials for MS analysis. All samples were n=3.

For targeted cardiolipin measurements, 24, of each prepared sample was injected into a 6410 Agilent Triple Quad LC-MS/MS system for analysis using an electrospray ionization source and negative ionization mode. Chromatographic separation was achieved on an Agilent 300 SB-C8 RRHD column (1.8 μm, 2.1×50 mm). The mobile phase A was 10 mM ammonium acetate in acetonitrile/H₂O (6:4, v/v), and mobile phase B was 10 mM ammonium acetate in isopropyl alcohol/acetonitrile/H₂O (90:10:1, v/v/v). The mobile phase composition changed from 60% A to 1% A over the 12 minute separation, followed by a rapid increase to 60% A and equilibration to prepare for the next injection. The total experimental time for each injection was 20 minutes. The flow rate was 0.26 mL/min, the auto-sampler temperature was 4° C., and the column compartment temperature was set to 55° C. Targeted MS/MS data were acquired using multiple-reaction-monitoring (MRM) mode. MassHunter Workstation Software Quantitative Analysis for QQQ B.07.00 (Agilent) was used to integrate extracted MRM peaks.

Untargeted Lipidomic Analysis

1 million cells were extracted with 225 μl of methanol at −20° C. containing an internal standard mixture of PE (17:0/17:0), PG (17:0/17:0), PC (17:0/0:0), C17 sphingosine, ceramide (d18:1/17:0), SM (d18:0/17:0), palmitic acid-d₃, PC (12:0/13:0), cholesterol-d₇, TG (17:0/17:1/17:0)-d₅, DG (12:0/12:0/0:0), DG (18:1/2:0/0:0), MG (17:0/0:0/0:0), PE (17:1/0:0), LPC (17:0), LPE (17:1), and 750 μL of MTBE (methyl tertiary butyl ether) (Sigma Aldrich) at −20° C. containing the internal standard cholesteryl ester 22:1. Cells were vortexed for 20 sec, sonicated for 5 min and shaken for 6 min at 4° C. with an Orbital Mixing Chilling/Heating Plate (Torrey Pines Scientific Instruments). Then 188 μl of LC-MS grade water (Fisher) was added. Samples were vortexed, centrifuged at 14,000 rcf (Eppendorf 5415D). The upper (non-polar, organic) phase was collected in two 350 μL aliquots and evaporated to dryness. One organic phase aliquot was re-suspended in 100 μL of methanol:toluene (9:1, v/v) mixture containing 50 ng/mL CUDA ((12-[[(cyclohexylamino)carbonyl]amino]-dodecanoic acid) (Cayman Chemical). Samples were then vortexed, sonicated for 5 min and centrifuged at 16,000 rcf and prepared for lipidomic analysis. Method blanks and pooled human plasma (BioreclamationlVT) were included as quality control samples. WT FA CM, HADHA Mut 12D FA were n=2, HADHA KO CM were n=3 and HADHA Mut 6D FA were n=2 with 6 technical replicates.

Chromatographic and Mass Spectrometric Conditions for Lipidomic RPLC-QTOF Analysis

Re-suspended samples were injected at 3 μL and 5 μL for ESI positive and negative modes, respectively, onto a Waters Acquity UPLC CSH C18 (100 mm length×2.1 mm id; 1.7 μm particle size) with an additional Waters Acquity VanGuard CSH C18 pre-column (5 mm×2.1 mm id; 1.7 μm particle size) maintained at 65° C. was coupled to a Vanquish UHPLC System. To improve lipid coverage, different mobile phase modifiers were used for positive and negative mode analysis [Cajka, T. and O. Fiehn, Increasing lipidomic coverage by selecting optimal mobile phase-modifiers in LC-MS of blood plasma. Metabolomics, 2016. 12(2): p. 34]. For positive mode 10 mM ammonium formate and 0.1% formic acid were used and 10 mM ammonium acetate (Sigma-Aldrich) was used for negative mode. Both positive and negative modes used the same mobile phase composition of (A) 60:40 v/v acetonitrile:water (LC-MS grade) and (B) 90:10 v/v isopropanol:acetonitrile. The gradient started at 0 min with 15% (B), 0-2 min 30% (B), 2-2.5 min 48% (B), 2.5-11 min 82% (B), 11-11.5 min 99% (B), 11.5-12 min 99% (B), 12-12.1 min 15% (B), and 12.1-15 min 15% (B). A flow rate of 0.6 mL/min was used. For data acquisition a Q-Exactive HF Hybrid Quadrupole-Orbitrap Mass Spectrometer was used with the following parameters: mass range, m/z 100-1200; MS' resolution 60,000: data-dependent MS² resolution 15,000; NCE 20, 30, 40; 4 targets/MS' scan; gas temperature 369° C., sheath gas flow (nitrogen), 60 units, aux gas flow 25 units, sweep gas flow 2 units; spray voltage 3.59 kV.

LC-MS Data Processing Using MS-DIAL and Statistics

Untargeted lipidomic data processing was performed using MS-DIAL [Tsugawa, H., et al., MS-DIAL: data-independent MS/MS deconvolution for comprehensive metabolome analysis. Nat Methods, 2015. 12(6): p. 523-6] for deconvolution, peak picking, alignment, and identification. In house m/z and retention time libraries were used in addition to MS/MS spectra databases in msp format [Kind, T., et al., LipidBlast in silico tandem mass spectrometry database for lipid identification. Nat Methods, 2013. 10(8): p. 755-8]. Features were reported when present in at least 50% of samples in each group. Statistical analysis was done by first normalizing data using the sum of the knowns, or mTIC normalization, to scale each sample. Normalized peak heights were then submitted to R for statistical analysis. ANOVA analysis was performed with FDR correction and post hoc testing.

### Exemplary Embodiments

For purposes of illustration only, a non-limiting listing of exemplary embodiments encompassed by the disclosure includes:

A1. A method for inducing maturation of cardiomyocyte, comprising inducing in an immature cardiomyocyte two or more of the following: overexpression of a Let7i microRNA (miRNA), overexpression of miR-452, reduced expression of miR-122, and reduced expression of miR-200a.

A2. The method of embodiment A1, comprising inducing in an immature cardiomyocyte three or more of the following: overexpression of a Let7i miRNA, overexpression of miR-452, reduced expression of miR-122, and reduced expression of miR-200a.

A3. The method of embodiment A1 or A2, comprising inducing in an immature cardiomyocyte overexpression of a Let7i miRNA, overexpression of miR-452, reduced expression of miR-122, and reduced expression of miR-200a.

A4. The method of one of embodiments A1-A3, wherein inducing overexpression comprises contacting the immature cardiomyocyte with a vector comprising a nucleic acid encoding the miRNA to be overexpressed.

A5. The method of embodiment A4, wherein the vector is configured to promote transient expression of the nucleic acid encoding the miRNA to be overexpressed.

A6. The method of embodiment A4 or A5, wherein the vector is a viral vector configured to integrate the nucleic acid encoding the miRNA to be overexpressed into the genome of the immature cardiomyocyte.

A7. The method of one of embodiments A4-A6, wherein the viral vector is a lentiviral vector or an adeno-associated viral vector.

A8. The method of one of embodiments A1-A7, wherein inducing reduced expression of an miRNA comprises contacting the immature cardiomyocyte with a nucleic acid fragment that hybridizes to the miRNA targeted for reduced expression, or with a vector comprising a nucleic acid encoding a transcript that hybridizes to the miRNA targeted for reduced expression.

A9. The method of one of embodiments A1-A8, wherein inducing reduced expression comprises implementing a knockout of a gene encoding the miRNA.

A10. The method of one of embodiments A1-A9, wherein inducing reduced expression comprises providing the immature cardiomyocyte with nuclease enzyme and a guide nucleic acid with a sequence to facilitate the specific cleavage of a nucleic acid encoding the miRNA targeted for reduced expression by the nuclease enzyme.

A11. The method of one of embodiments A1-A10, wherein providing the providing the immature cardiomyocyte with a nuclease enzyme comprises contacting the immature cardiomyocyte with the nuclease enzyme or with a vector encoding the nuclease enzyme, wherein the vector is configured to promote expression of the enzyme in the cardiomyocyte.

A12. The method of embodiment A10, wherein providing the providing the immature cardiomyocyte with a guide nucleic acid comprises contacting the immature cardiomyocyte with the guide nucleic acid or with a vector encoding the guide nucleic acid, wherein the vector is configured to promote expression of the guide nucleic acid in the cardiomyocyte.

A13. The method of embodiment A10 or A11, wherein the nuclease enzyme is an endonuclease, such as Cas9 or TALENS.

A14. The method of one of embodiments A8-A12 wherein the vector is a viral vector.

A15. The method of embodiment A14, where the viral vector is a lentiviral vector or an adeno-associated viral vector.

A16. The method of one of embodiments A1-A15, wherein the immature cardiomyocyte is derived from a stem cell.

A17. The method of one of embodiments A1-A16, wherein the immature cardiomyocyte is derived from a stem cell in vitro.

A18. The method of embodiments A16 or A17, wherein the stem cell is an embryonic stem cell, pluripotent stem cell, or induced pluripotent stem cell.

A19. The method of one of embodiments A1-A18, further comprising contacting the immature cardiomyocyte with two or more long-chain fatty acids selected from palmitic acid, oleic acid, and linoleic acid.

A20. The method of embodiment A19, wherein the one or more long chain fatty acids comprise palmitate, oleic acid, and linoleic acid.

A21. The method of one of embodiments A1-A20, wherein the cardiomyocyte comprises a genetic aberration.

A22. The method of embodiment A21, wherein the genetic aberration is associated with a metabolic or pathological disease state in the heart.

A23. The method of embodiment A22, wherein the genetic aberration is associated with a fatty acid oxidation (FAO) disorder.

A24. The method of embodiment A22 or A23, wherein the cardiomyocyte comprises a mutation in a gene encoding one of the following: HADHA, FATP1, FACS1, OCTN2, L-CPTI, M-CPT I, CAT, CPT II, VLCAD, LCAD, MCAD, SCAD, LCHAD, SHYD, M/SCHAD, SKAT, MKAT, HS, HL, ETF, and ETF QO.

B1. The cardiomyocyte produced by any method recited in one of embodiments A1-A24.

B2. The cardiomyocyte of embodiment B1, wherein the cardiomyocyte comprises a genetic aberration.

B3. The cardiomyocyte of embodiment B2, wherein the genetic aberration is associated with a fatty acid oxidation (FAO) disorder.

B4. The cardiomyocyte of embodiment B3, wherein the genetic aberration is a mutation in the gene encoding HADHA.

C1. A method of treating a subject with a condition treatable by administration of cardiomyocytes with a mature cardiolipin profile, comprising administering to the subject an effective amount of cardiomyocytes as recited in embodiment B1.

C2. The method of embodiment C1, wherein the subject has compromised cardiac tissue or cells.

C2. The method of embodiment C1 or C2, wherein the subject has diabetes, congenital heart disease, ischemia, myopathy, mitochondrial disease, and/or has suffered from infarction.

C3. The method of one of embodiments C1-C3, wherein the mitochondrial disease is a fatty acid oxidation (FAO) disorder.

C4. The method of one of embodiments C1-C4, wherein the subject has a mutation in the gene encoding HADHA.

C5. The method of one of embodiments C1-C5, wherein the subject experiences arrhythmia.

C6. The method of one of embodiments C1-C6, wherein the subject is at an elevated risk of sudden infant death syndrome (SIDS).

D1. A method of screening a compound for modulation of heart function, comprising:

contacting one or more cardiomyocytes as recited in one of embodiments B1-B4 with a candidate agent; and

measuring a cardiac functional parameter in the one or more cardiomyocytes;

wherein a change in the cardiac functional parameter indicates the candidate agent modulates heart function.

D2. The method of embodiment D1, wherein the mature cardiomyocyte comprises a genetic aberration.

D3. The method of embodiment D1 or D2, wherein the genetic aberration is associated with a fatty acid oxidation (FAO) disorder.

D4. The method of one of embodiments D1-D3, wherein the genetic aberration is a mutation in the gene encoding HADHA.

D5. The method of one of embodiments D1-D4, wherein the cardiac functional parameter comprises lipid profile, cardiolipin profile, metabolic profile, oxygen consumption rate, mitochondrial proton gradient, force of contraction, calcium transport, conduction velocity, glucose stress, and cell death.

E1. A method of treating a mitochondrial fatty acid oxidation (FAO) disorder in a subject, the method comprising administering an effective amount of a composition stabilizing a cardiolipin profile or promoting mature cardiolipin remodeling in mitochondria of the subject.

E2. The method of embodiment E1, wherein the FAO disorder is associated with diabetes, heart failure, neurodegeneration, advanced age, congenital heart disease, ischemia, myopathy, and/or instance of infarction.

E3. The method of embodiment E1 or E2, wherein the FAO disorder is a fatty acid (FA) β-oxidation disorder.

E4. The method of one of embodiments E1-E3, wherein a phenotype of the mitochondrial dysfunction is associated with increased risk of sudden infant death syndrome.

E5. The method of one of embodiments E1-E4, wherein stabilizing a cardiolipin profile comprises prevention of oxidation of cardiolipin.

E6. The method of one of embodiments E1-E4, wherein the composition is or comprises elamipretide.

F1. A method of detecting the pathological state of a cultured cardiomyocyte comprising,

determining the cardiolipin profile in the cardiomyocyte, wherein a relative increase of cardiolipins with acyl chains with more than 18 carbons indicates and a relative decrease in cardiolipins with acyl chains with less than 18 carbons indicates a reduced pathological state of the cardiomyocyte.

F2. The method of embodiment F1, wherein the increase or decrease of cardiolipins is relative to a wild-type immature cardiomyocyte.

F3. The method of embodiment F1 or F2, wherein the cultured cardiomyocyte is derived from a stem cell in vitro.

F4. The method of embodiment F3, wherein the stem cell is an embryonic stem cell, pluripotent stem cell, or induced pluripotent stem cell.

F5. The method of one of embodiments F1-F4, wherein the pathological state is associated with a mitochondrial dysfunction.

F6. The method of embodiment F5, wherein the mitochondrial dysfunction is mitchondrial tri-functional protein deficiency.

F7. The method of one of embodiments F1-F6, further comprising contacting the cultured cardiomyocyte with a candidate agent for reducing the pathological state of the cultured cardiomyocyte.

F8. The method of embodiment F7, comprising determining the cardiolipin profile in the cultured cardiomyocyte a plurality of times before, during, and/or after the step of contacting the cultured cardiomyocyte with a candidate agent to ascertain the effect of the candidate agent on the pathological state of the cultured cardiomyocyte.

G1. A composition to induce maturation of a cultured cardiomyocyte, comprising two or more of the following: a nucleic acid construct encoding a Let7i microRNA, a nucleic acid construct encoding miR-452, a nucleic acid construct that is or encodes an oligomer that hybridizes to a portion of a sequence encoding miR-122, and a nucleic acid construct that is or encodes an oligomer that hybridizes to a portion of a sequence encoding miR-200a.

G2. The composition of embodiment G1, comprising three or more of the following: a nucleic acid construct encoding a Let7i microRNA, a nucleic acid construct encoding miR-452, a nucleic acid construct that is or encodes an oligomer that hybridizes to a portion of a sequence encoding miR-122, and a nucleic acid construct that is or encodes an oligomer that hybridizes to a portion of a sequence encoding miR-200a.

G3. The composition of embodiment G1 or G2, comprising a nucleic acid construct encoding a Let7i microRNA, a nucleic acid construct encoding miR-452, a nucleic acid construct that is or encodes an oligomer that hybridizes to a portion of a sequence encoding miR-122, and a nucleic acid construct that is or encodes an oligomer that hybridizes to a portion of a sequence encoding miR-200a.

G4. The composition of one of embodiments G1-G3, wherein the nucleic acid constructs that encode a microRNA and/or encode an oligomer are each operatively linked to one or more promoter sequences.

G5. The composition of one of embodiments G1-G4, wherein one or more of the constructs are incorporated into one or more vectors configured for delivery to a cell.

G6. The composition of embodiment G5, wherein the one or more vectors are viral vectors.

G7. The composition of embodiment G5 or G660, wherein at least one viral vector is a lentiviral vector or AAV vector.

G8. The composition of one of embodiments G1-G7, wherein the oligomer that hybridizes to a portion of a sequence encoding miR-122 and the oligomer that hybridizes to a portion of a sequence encoding miR-200a are guide RNA molecules that are configured to induce a gene editing enzyme to cleave miR-122 and miR-200a, respectively.

G9. The composition of embodiment G8, wherein the gene editing enzyme is a nuclease.

G10. The composition of one of embodiments G1-G9, further comprising a nuclease.

G11. The composition of embodiment G9 or G10, wherein the nuclease is Cas9.

G12. The composition of one of embodiments G1-G11, further comprising one or more long-chain fatty acids.

G13. The composition of embodiment G12, wherein the one or more long-chain fatty acids comprise two or more of palmitate, oleic acid, and linoleic acid.

G14. The composition of embodiment G12 or G13, wherein the one or more long-chain fatty acids comprise palmitate, oleic acid, and linoleic acid.

H1. A kit comprising the composition or compositions of embodiment G1-G14.

H2. The kit of embodiment H1, further comprising cell culture media and/or one or more immature cardiomyocytes.

While illustrative embodiments have been illustrated and described, it will be appreciated that various changes can be made therein without departing from the spirit and scope of the invention.

1. A method for inducing maturation of cardiomyocyte, comprising inducing in an immature cardiomyocyte two or more of the following: overexpression of a Let7i microRNA (miRNA), overexpression of miR-452, reduced expression of miR-122, and reduced expression of miR-200a.

2. The method of claim 1, comprising inducing in an immature cardiomyocyte three or more of the following: overexpression of a Let7i miRNA, overexpression of miR-452, reduced expression of miR-122, and reduced expression of miR-200a.

3. The method of claim 2, comprising inducing in an immature cardiomyocyte overexpression of a Let7i miRNA, overexpression of miR-452, reduced expression of miR-122, and reduced expression of miR-200a.

4. The method of one of claims 1-3, wherein inducing overexpression comprises contacting the immature cardiomyocyte with a vector comprising a nucleic acid encoding the miRNA to be overexpressed.

5. The method of claim 4, wherein the vector is configured to promote transient expression of the nucleic acid encoding the miRNA to be overexpressed.

6. The method of claim 4, wherein the vector is a viral vector configured to integrate the nucleic acid encoding the miRNA to be overexpressed into the genome of the immature cardiomyocyte.

7. The method of claim 6, wherein the viral vector is a lentiviral vector or an adeno-associated viral vector.

8. The method of one of claims 1-3, wherein inducing reduced expression of an miRNA comprises contacting the immature cardiomyocyte with a nucleic acid fragment that hybridizes to the miRNA targeted for reduced expression, or with a vector comprising a nucleic acid encoding a transcript that hybridizes to the miRNA targeted for reduced expression.

9. The method of claim 1, wherein inducing reduced expression comprises implementing a knockout of a gene encoding the miRNA.

10. The method of one of claims 1-3, wherein inducing reduced expression comprises providing the immature cardiomyocyte with nuclease enzyme and a guide nucleic acid with a sequence to facilitate the specific cleavage of a nucleic acid encoding the miRNA targeted for reduced expression by the nuclease enzyme.

11. The method of claim 10, wherein providing the providing the immature cardiomyocyte with a nuclease enzyme comprises contacting the immature cardiomyocyte with the nuclease enzyme or with a vector encoding the nuclease enzyme, wherein the vector is configured to promote expression of the enzyme in the cardiomyocyte.

12. The method of claim 10, wherein providing the providing the immature cardiomyocyte with a guide nucleic acid comprises contacting the immature cardiomyocyte with the guide nucleic acid or with a vector encoding the guide nucleic acid, wherein the vector is configured to promote expression of the guide nucleic acid in the cardiomyocyte.

13. The method of claim 10, wherein the nuclease enzyme is an endonuclease, such as Cas9 or TALENS.

14. The method of one of claim 8, 11, or 12 wherein the vector is a viral vector.

15. The method of claim 14, where the viral vector is a lentiviral vector or an adeno-associated viral vector.

16. The method of claim 1, wherein the immature cardiomyocyte is derived from a stem cell.

17. The method of claim 14, wherein the immature cardiomyocyte is derived from a stem cell in vitro.

18. The method of claim 16 or claim 17, wherein the stem cell is an embryonic stem cell, pluripotent stem cell, or induced pluripotent stem cell.

19. The method of one of claims 1-18, further comprising contacting the immature cardiomyocyte with two or more long-chain fatty acids selected from palmitic acid, oleic acid, and linoleic acid.

20. The method of claim 19, wherein the one or more long chain fatty acids comprise palmitate, oleic acid, and linoleic acid.

21. The method of one of claims 1-20, wherein the cardiomyocyte comprises a genetic aberration.

22. The method of claim 21, wherein the genetic aberration is associated with a metabolic or pathological disease state in the heart.

23. The method of claim 22, wherein the genetic aberration is associated with a fatty acid oxidation (FAO) disorder.

24. The method of claim 22, wherein the cardiomyocyte comprises a mutation in a gene encoding one of the following: HADHA, FATP1, FACS1, OCTN2, L-CPTI, M-CPT I, CAT, CPT II, VLCAD, LCAD, MCAD, SCAD, LCHAD, SHYD, M/SCHAD, SKAT, MKAT, HS, HL, ETF, and ETF QO.

25. A cardiomyocyte produced by any method recited in one of claims 1-24.

26. The cardiomyocyte of claim 25, wherein the cardiomyocyte comprises a genetic aberration.

27. The cardiomyocyte of claim 26, wherein the genetic aberration is associated with a fatty acid oxidation (FAO) disorder.

28. The cardiomyocyte of claim 27, wherein the genetic aberration is a mutation in the gene encoding HADHA.

29. A method of treating a subject with a condition treatable by administration of cardiomyocytes with a mature cardiolipin profile, comprising administering to the subject an effective amount of cardiomyocytes as recited in claim 25.

30. The method of claim 29, wherein the subject has compromised cardiac tissue or cells.

31. The method of claim 29, wherein the subject has diabetes, congenital heart disease, ischemia, myopathy, mitochondrial disease, and/or has suffered from infarction.

32. The method of claim 29, wherein the mitochondrial disease is a fatty acid oxidation (FAO) disorder.

33. The method of claim 29, wherein the subject has a mutation in the gene encoding HADHA.

34. The method of claim 29, wherein the subject experiences arrhythmia.

35. The method of claim 29, wherein the subject is at an elevated risk of sudden infant death syndrome (SIDS).

36. A method of screening a compound for modulation of heart function, comprising:
contacting one or more cardiomyocytes as recited in one of claims 25-28 with a candidate agent; and
measuring a cardiac functional parameter in the one or more cardiomyocytes;
wherein a change in the cardiac functional parameter indicates the candidate agent modulates heart function.

37. The method of claim 36, wherein the mature cardiomyocyte comprises a genetic aberration.

38. The method of claim 37, wherein the genetic aberration is associated with a fatty acid oxidation (FAO) disorder.

39. The method of claim 38, wherein the genetic aberration is a mutation in the gene encoding HADHA.

40. The method of claim 36, wherein the cardiac functional parameter comprises lipid profile, cardiolipin profile, metabolic profile, oxygen consumption rate, mitochondrial proton gradient, force of contraction, calcium transport, conduction velocity, glucose stress, and cell death.

41. A method of treating a mitochondrial fatty acid oxidation (FAO) disorder in a subject, the method comprising administering an effective amount of a composition stabilizing a cardiolipin profile or promoting mature cardiolipin remodeling in mitochondria of the subject.

42. The method of claim 41, wherein the FAO disorder is associated with diabetes, heart failure, neurodegeneration, advanced age, congenital heart disease, ischemia, myopathy, and/or instance of infarction.

43. The method of claim 41, wherein the FAO disorder is a fatty acid (FA) β-oxidation disorder.

44. The method of claim 41, wherein a phenotype of the mitochondrial dysfunction is associated with increased risk of sudden infant death syndrome.

45. The method of claim 41, wherein stabilizing a cardiolipin profile comprises prevention of oxidation of cardiolipin.

46. The method of claims 41-45, wherein the composition is or comprises elamipretide.

47. A method of detecting the pathological state of a cultured cardiomyocyte comprising,
determining the cardiolipin profile in the cardiomyocyte, wherein a relative increase of cardiolipins with acyl chains with more than 18 carbons indicates and a relative decrease in cardiolipins with acyl chains with less than 18 carbons indicates a reduced pathological state of the cardiomyocyte.

48. The method of claim 47, wherein the increase or decrease of cardiolipins is relative to a wild-type immature cardiomyocyte.

49. The method of claim 47, wherein the cultured cardiomyocyte is derived from a stem cell in vitro.

50. The method of claim 49, wherein the stem cell is an embryonic stem cell, pluripotent stem cell, or induced pluripotent stem cell.

51. The method of claim 47, wherein the pathological state is associated with a mitochondrial dysfunction.

52. The method of claim 51, wherein the mitochondrial dysfunction is mitchondrial tri-functional protein deficiency.

53. The method of claim 47, further comprising contacting the cultured cardiomyocyte with a candidate agent for reducing the pathological state of the cultured cardiomyocyte.

54. The method of claim 53, comprising determining the cardiolipin profile in the cultured cardiomyocyte a plurality of times before, during, and/or after the step of contacting the cultured cardiomyocyte with a candidate agent to ascertain the effect of the candidate agent on the pathological state of the cultured cardiomyocyte.

55. A composition to induce maturation of a cultured cardiomyocyte, comprising two or more of the following: a nucleic acid construct encoding a Let7i microRNA, a nucleic acid construct encoding miR-452, a nucleic acid construct that is or encodes an oligomer that hybridizes to a portion of a sequence encoding miR-122, and a nucleic acid construct that is or encodes an oligomer that hybridizes to a portion of a sequence encoding miR-200a.

56. The composition of claim 55, comprising three or more of the following: a nucleic acid construct encoding a Let7i microRNA, a nucleic acid construct encoding miR-452, a nucleic acid construct that is or encodes an oligomer that hybridizes to a portion of a sequence encoding miR-122, and a nucleic acid construct that is or encodes an oligomer that hybridizes to a portion of a sequence encoding miR-200a.

57. The composition of claim 55, comprising a nucleic acid construct encoding a Let7i microRNA, a nucleic acid construct encoding miR-452, a nucleic acid construct that is or encodes an oligomer that hybridizes to a portion of a sequence encoding miR-122, and a nucleic acid construct that is or encodes an oligomer that hybridizes to a portion of a sequence encoding miR-200a.

58. The composition of one of claims 55-57, wherein the nucleic acid constructs that encode a microRNA and/or encode an oligomer are each operatively linked to one or more promoter sequences.

59. The composition of one of claims 55-57, wherein one or more of the constructs are incorporated into one or more vectors configured for delivery to a cell.

60. The composition of claim 59, wherein the one or more vectors are viral vectors.

61. The composition of claim 60, wherein at least one viral vector is a lentiviral vector or AAV vector.

62. The composition of one of claims 55-61, wherein the oligomer that hybridizes to a portion of a sequence encoding miR-122 and the oligomer that hybridizes to a portion of a sequence encoding miR-200a are guide RNA molecules that are configured to induce a gene editing enzyme to cleave miR-122 and miR-200a, respectively.

63. The composition of claim 62, wherein the gene editing enzyme is a nuclease.

64. The composition of one of claims 55-63, further comprising a nuclease.

65. The composition of claim 63 or claim 64, wherein the nuclease is Cas9.

66. The composition of one of claims 55-65, further comprising one or more long-chain fatty acids.

67. The composition of claim 66, wherein the one or more long-chain fatty acids comprise two or more of palmitate, oleic acid, and linoleic acid.

68. The composition of claim 67, wherein the one or more long-chain fatty acids comprise palmitate, oleic acid, and linoleic acid.


SEQUENCE LISTING (46 sequences)

SEQ ID NO: 1 - DNA, 90 bp, Homo sapiens
atggtggcctgccgggcgattggcatcctcagccgcttttctgccttcaggatcctccgctcccgaggtgaggcctggccgagggcatcc

SEQ ID NO: 2 - protein, 43 aa, Homo sapiens
MVACRAIGILSRFSAFRILRSRGYICRNFTGSSALLTRTHINY

SEQ ID NO: 3 - DNA, 68 bp, Artificial Sequence
  Synthetic
atggtggcctgccgggcgattggcatcctcagccgcttttctgccttcaggatcctccgagggcatcc

SEQ ID NO: 4 - protein, 37 aa, Artificial Sequence
  Synthetic
MVACRAIGILSRFSAFRILLYMPQFYRVFCFADQNPY

SEQ ID NO: 5 - DNA, 79 bp, Homo sapiens
atggtggcctgccgggcgattggcatcctcagccgcttttctgccttcaggatcctccgctcccgaggtgaggcctggc

SEQ ID NO: 6 - protein, 43 aa, Homo sapiens; identical to SEQ ID NO: 2

SEQ ID NO: 7 - DNA, 75 bp, Artificial Sequence
  Synthetic
ccgggcgattggcatcctcagccgcttttctgccttcaggatcctccgcggaggtgaggccaggtgaggcctggc

SEQ ID NO: 8 - protein, 43 aa, Artificial Sequence
  Synthetic
MVACRAIGILSRFSAFRILRGGEARLYMPQFYRVFCFADQNPY

SEQ ID NO: 9 - DNA, 78 bp, Artificial Sequence
  Synthetic
gtggcctgccgggcgattggcatcctcagccgcttttctgccttcaggatcctccgctccgcggaggtgaggcctggc

SEQ ID NO: 10 - protein, 36 aa, Artificial Sequence
  Synthetic
MVACRAIGILSRFSAFRILRSAEVIYAAILQGLLLC

SEQ ID NO: 11 - DNA, 84 bp, Homo sapiens
ctggctgaggtagtagtttgtgctgttggtcgggttgtgacattgcccgctgtggagataactgcgcaagctactgccttgcta

SEQ ID NO: 12 - DNA, 14 bp, Artificial Sequence
  Synthetic
tccgcgtggtcccg

SEQ ID NO: 13 - DNA, 17 bp, Artificial Sequence
  Synthetic
attgtcctccgcggcgc

SEQ ID NO: 14 - DNA, 85 bp, Homo sapiens
gctaagcacttacaactgtttgcagaggaaactgagactttgtaactatgtctcagtctcatctgcaaagaagtaagtgctttgc

SEQ ID NO: 15 - DNA, 25 bp, Artificial Sequence
  Synthetic
caccgccggagtctgtctcataccc

SEQ ID NO: 16 - DNA, 25 bp, Artificial Sequence
  Synthetic
aaacgggtatgagacagactccggc

SEQ ID NO: 17 - DNA, 25 bp, Artificial Sequence
  Synthetic
caccgagcttgactctaacactgtc

SEQ ID NO: 18 - DNA, 25 bp, Artificial Sequence
  Synthetic
aaacgacagtgttagagtcaagctc

SEQ ID NO: 19 - DNA, 24 bp, Artificial Sequence
  Synthetic
caccgagtttccttagcagagctg

SEQ ID NO: 20 - DNA, 24 bp, Artificial Sequence
  Synthetic
aaaccagctctgctaaggaaactc

SEQ ID NO: 21 - DNA, 25 bp, Artificial Sequence
  Synthetic
caccgggcaggcctcacctcgggag

SEQ ID NO: 22 - DNA, 25 bp, Artificial Sequence
  Synthetic
aaacctcccgaggtgaggcctgccc

SEQ ID NO: 23 - DNA, 25 bp, Artificial Sequence
  Synthetic
caccgggaaggcagaaaagcggctg

SEQ ID NO: 24 - DNA, 25 bp, Artificial Sequence
  Synthetic
aaaccagccgcttttctgccttccc

SEQ ID NO: 25 - DNA, 20 bp, Artificial Sequence
  Synthetic
cgtacgtgttctgcacagcc

SEQ ID NO: 26 - DNA, 20 bp, Artificial Sequence
  Synthetic
cagcaatgttctgaaggccc

SEQ ID NO: 27 - DNA, 20 bp, Artificial Sequence
  Synthetic
cctgcccccttcaaggtaag

SEQ ID NO: 28 - DNA, 20 bp, Artificial Sequence
  Synthetic
ctggtctgataggtggggga

SEQ ID NO: 29 - DNA, 20 bp, Artificial Sequence
  Synthetic
gagagctaggctttgtgcca

SEQ ID NO: 30 - DNA, 20 bp, Artificial Sequence
  Synthetic
cttatgtggccccgtgttct

SEQ ID NO: 31 - DNA, 24 bp, Artificial Sequence
  Synthetic
tgtccctaccttgtctgttagcca

SEQ ID NO: 32 - DNA, 24 bp, Artificial Sequence
  Synthetic
attggaacatggcctctggatgga

SEQ ID NO: 33 - DNA, 20 bp, Artificial Sequence
  Synthetic
tggttcggtgcatgaaggac

SEQ ID NO: 34 - DNA, 21 bp, Artificial Sequence
  Synthetic
gtcgatgtagccatcagcatt

SEQ ID NO: 35 - DNA, 19 bp, Artificial Sequence
  Synthetic
ctgggctacactgagcacc

SEQ ID NO: 36 - DNA, 21 bp, Artificial Sequence
  Synthetic
aagtggtcgttgagggcaatg

SEQ ID NO: 37 - DNA, 21 bp, Artificial Sequence
  Synthetic
caacgcagacctgatggattt

SEQ ID NO: 38 - DNA, 19 bp, Artificial Sequence
  Synthetic
agcccccgcttcttcattc

SEQ ID NO: 39 - DNA, 26 bp, Artificial Sequence
  Synthetic
ccggtgagttctgagcagcctgactt

SEQ ID NO: 40 - DNA, 26 bp, Artificial Sequence
  Synthetic
atcctctgcctgatgttctcgaattc

SEQ ID NO: 41 - DNA, 31 bp, Artificial Sequence
  Synthetic
aaaaaaccggtctcacacgagctccattccc

SEQ ID NO: 42 - DNA, 31 bp, Artificial Sequence
  Synthetic
aaaaagaattccaaccccagttggtaagcgt

SEQ ID NO: 43 - DNA, 20 bp, Artificial Sequence
  Synthetic
actaggacgagctagtgggg

SEQ ID NO: 44 - DNA, 24 bp, Artificial Sequence
  Synthetic
acccaaagtgtacaatcattgact

SEQ ID NO: 45 - DNA, 90 bp, Homo sapiens
ccgggcccctgtgagcatcttaccggacagtgctggatttcccagcttgactctaacactgtctggtaacgatgttcaaaggtgacccgc

SEQ ID NO: 46 - DNA, 85 bp, Homo sapiens
ccttagcagagctgtggagtgtgacaatggtgtttgtgtctaaactatcaaacgccattatcacactaaatagctactgctaggc